(19) United States
(12) Patent Application Publication
(10) Pub. No.: US 2013/0324520 A1
(43) Pub. Date: Dec. 5, 2013

(54) RXRG MODULATORS FOR THE TREATMENT OF CANCER

(71) Applicants: Xiaoliang Xu, New York, NY (US); Suresh Jhanwar, New York, NY (US)

(72) Inventors: Xiaoliang Xu, New York, NY (US); Suresh Jhanwar, New York, NY (US)

(73) Assignee: SLOAN-KETTERING INSTITUTE FOR CANCER RESEARCH, New York, NY (US)

(21) Appl. No.: 13/985,421

(22) PCT Filed: Feb. 14, 2012
(86) PCT No.: PCT/US12/25137

§ 371 Date: Aug. 14, 2013

Related U.S. Application Data
(60) Provisional application No. 61/442,699, filed on Feb. 14, 2011.

(51) Int. Cl. A61K 31/551; A61K 31/192

(52) U.S. Cl. CPC A61K 31/551; A61K 31/192
USPC 514/220; 514/569; 435/375

(57) ABSTRACT

The present invention provides methods for treating cancer using modulators of retinoid X receptor gamma (RXRG). The ability of RXRG antagonists to disrupt the association of complexes comprising RXRG is demonstrated.

### CROSS-REFERENCE TO RELATED APPLICATIONS

This application claims priority to U.S. provisional application Ser. No. 61/442,699, filed Feb. 14, 2011, the entirety of which is hereby incorporated herein by reference.

### BACKGROUND

A variety of human cancers are observed to have alterations in the retinoblastoma protein (RB1). Cancers with RB1 mutations are often from mesenchymal cells or neuroendocrine cells and include retinoblastoma, small cell lung cancer (SCLC), bladder carcinoma, osteosarcoma, myeloma, liposarcoma, histiocytoma, leiomyosarcoma, and rhabdomyosarcoma.

Other cancers generally do not have RB1 mutation; some of them have RB1 gain and amplification. Such cancers include adenosarcoma (such as colorectal cancer, gastric cancer, non-small cell lung cancer (NSCLC), breast cancer, and pancreatic cancer, etc.), melanoma, myeloid leukemia, and neuroblastoma, to name but a few. Many of these cancers show MEK/ERK activation by activated mutation of KRAS, EGFR, HER2, PDGFR, C-RAF, etc. As is well known, there is a need to identify practical and relevant targets for the treatment of cancers, and a further need for the identification of cancer therapies.

Some cancers, such as certain colon, pancreatic, lung (NSCLC), breast, gastric, hepatocellular carcinoma, squamous carcinoma, thyroid cancer, and some leukemia often have KRAS or EGFR mutation, or HER2 activation.

Some other cancers may not have KRAS or EGFR mutations. This group of cancers generally have different mutations; for examples, retinoblastoma has RB1 mutation; melanoma often has BRAF mutation; some lung cancers have EML4-ALK fusion; cervical cancers often have HPV infection and Rb inactivation; some breast cancers have PIK3CA mutation without KRAS, EGFR or PTEN mutations, or HER2 activation. Prostate cancers, small cell lung cancer, breast cancer, glioma, and melanoma often have PTEN mutation, which often exhibit RB1 or BRAF mutation.

Lung cancer is one of the most devastating cancers and the most common cancers in the world. It is the leading cause of cancer death in the United States. There are two main types of lung cancers, non-small cell lung cancer (NSCLC) and small cell lung cancer (SCLC). Current complimentary treatment strategies such as radiotherapy and chemotherapy for lung cancer have much higher toxicity and adverse effects. Most patients develop metastasis and die in 1-3 years, even with combination of several treatments. The development of new alternative or complimentary therapies is urgent and vital for improving outcomes for these patients. Clarification of signaling pathway in lung cancer will help us to conquer this most devastating cancer. NSCLC tumorigenesis often exhibits KRAS or EGFR mutant activation and RB1 hyperphosphorylation, but not RB1 mutation. Recently we find that phospho-Rb is necessary for cell proliferation, which is hyper-phosphorylated and over-expressed in KRAS or EGFR mutant colon cancers and NSCLCs. In NSCLC, KRAS or EGFR mutation causes cyclin D1 activation and Rb hyperphosphorylation.

Pancreatic cancer is one of the most lethal human malignancies with a very high case fatality rate. Most patients present with late stage disease and the best currently available therapies have modest palliative impact on the disease course. To date, targeted therapies have also had limited impact in pancreas adenocarcinoma. Targeting mutated K-Ras has great attraction for this disease and heretofore approaches have been unsuccessful. We propose a novel method of targeting K-Ras.

EGFR-RAS-CRAF-MEK-ERK pathway is often activated in many cancers including NSCLC, colorectal, and pancreatic cancers. Their inhibitors have been used as the targeted therapy of some cancers with EGFR and BRAF mutation, but not KRAS mutation. Development of KRAS direct inhibitor has proven very difficult and almost no good KRAS inhibitors have been developed. MEK inhibitors usually have strong side effects: clinical trial of MEK inhibitor PD0325901 was terminated because of high toxicity in patients. PI3K pathway activation mediated resistance to MEK inhibitors in KRAS mutant cancers was often observed. Many KRAS mutant cancers, for example, NSCLC, colon cancer, and pancreatic cancers, are resistant to MEK inhibitor. The EGFR inhibitors and BRAF inhibitors are proved not effective for the treatment of KRAS mutant colorectal and lung cancers. There is an urgency to find new strategies for treating cancers with KRAS or EGFR mutations.

### SUMMARY OF THE INVENTION

The present invention encompasses the recognition that there exists a need for methods for treating patients with cancer.

In some embodiments, the present invention provides methods of treating cancer in a subject suffering therefrom comprising administering to the subject a therapeutically effective amount of a compound described herein. In some embodiments, a compound used in accordance with the provided method is a retinoid X receptor gamma (RXRG) antagonist. In some embodiments, the invention provides methods of treating a subject suffering from or susceptible to cancer with KRAS, EGFR, or PTEN mutations, by administering a RXRG antagonist.

In some embodiments, the invention provides methods of inhibiting growth of cancer cells with a compound described herein. In some embodiments, a compound used in accordance with the provided method is a retinoid X receptor gamma (RXRG) antagonist. In some embodiments, the invention provides methods of inhibiting growth of cancer cells with KRAS, EGFR, or PTEN mutations with a RXRG antagonist.

In certain embodiments, the invention provides methods of inhibiting proliferation of cancer cells with a compound described herein. In some embodiments, a compound used in accordance with the provided method is a retinoid X receptor gamma (RXRG) antagonist. In certain embodiments, the invention provides methods of inhibiting proliferation of cancer cells with KRAS, EGFR, or PTEN mutations with a RXRG antagonist.

In some embodiments, the invention provides methods of promoting apoptosis of cancer cells with a compound described herein. In some embodiments, a compound used in accordance with the provided method is a retinoid X receptor gamma (RXRG) antagonist. In some embodiments, the invention provides methods of promoting apoptosis of cancer cells with KRAS, EGFR, or PTEN mutations with a RXRG antagonist.

In some embodiments, the invention provides methods of suppressing G1/S transition in cancer cells with a compound described herein. In some embodiments, a compound used in accordance with the provided method is a retinoid X receptor gamma (RXRG) antagonist. In some embodiments, the invention provides methods of suppressing G1/S transition in cancer cells with KRAS, EGFR, or PTEN mutations with a RXRG antagonist.

In one aspect, the invention provides methods of treating a subject suffering from or susceptible to a cancer without KRAS, EGFR or PTEN mutations, with a retinoid X receptor gamma (RXRG) agonist.

In one aspect, the invention provides methods of inhibiting growth of cancer cells without KRAS, EGFR or PTEN mutations with a retinoid X receptor gamma (RXRG) agonist.

In one aspect, the invention provides methods of inhibiting proliferation of cancer cells without KRAS, EGFR or PTEN mutations with a retinoid X receptor gamma (RXRG) agonist.

In one aspect, the invention provides methods of promoting apoptosis of cancer cells without KRAS, EGFR or PTEN mutations with a retinoid X receptor gamma (RXRG) agonist.

In one aspect, the invention provides methods of delaying S phase progression and G2/M transition in cancer cells without KRAS, EGFR or PTEN mutations with a retinoid X receptor gamma (RXRG) agonist.

In some embodiments, the invention provides methods of modulating functions of Treprec-Xu complex (S phase promoting complex, SPC) in cancer. In certain embodiments, the invention provides methods of modulating functions of Treprec-Xu complex (SPC) in cancer by inhibiting or promoting association or dissociation of one or more components of the complex with each other and/or with the complex.

In some embodiments, the present invention describes the function of retinoid X receptor gamma (RXRG) in cancer. In one aspect, the present invention provides methods to modulate RXRG function in cancer.

### BRIEF DESCRIPTION OF THE DRAWING

FIG. 1. Co-IP results of S-phase promoting complex (SPC). Our Co-IP results and those in literature showed close interactions among G1-S proteins and G2-M proteins. S-phase promoting complex comprises THRB2, RXRG, PP2A, Phospho-Rb family proteins, cyclin E, Emi1, and CDK2. S-phase promoting complex can be targeted for therapy and used as efficient marker of cancer treatment.

FIG. 2. RXRG Co-IP in HCT116. HCT116 lysate was immuno-precipitated by antibodies listed above by Direct-Co-IP kit and detected by western blot with phospho-Rb-S807 and RXRG antibodies. RXRG binds to phosphorylated Rb, phospho-p107, Cyclin E, PP1, P53, TRB2, and TRB1.

FIG. 3. Co-localization of phospho-Rb with TRB2, PP2A, Cyclin E, RXRG, and Emi1 in S-phase promoting Complex in HCT116 as shown by co-immunofluorescence.

FIG. 4. Co-localization of phospho-Rb-S807 with cyclin E, and cyclin E with CDK2 in HCT116 cells as shown by co-immunofluorescence.

FIG. 5. Co-immunofluorescence of S-phase promoting complex showed co-localization of phospho-Rb-S807 with RXRG and cyclin E with RXRG in HCT116 cells.

FIG. 6. Co-immunofluorescence of S-phase promoting complex showed co-localization of phospho-Rb-S807 with Emi1 and cyclin E with Emi1 in HCT116 cells.

FIG. 7. Co-immunofluorescence of S-phase promoting complex showed co-localization of phospho-Rb-S807 with phospho-p107 and cyclin E with phospho-p107, in HCT116 cells.

FIG. 8. Co-immunofluorescence of S-phase promoting complex showed co-localization of phospho-Rb-S807 with phospho-p130 and cyclin E with phospho-p130 in HCT116 cells.

FIG. 9. Co-immunofluorescence of S-phase promoting complex showed co-localization of phospho-Rb-S807 with TRB2 and cyclin E with TRB2 in HCT116 cells.

FIG. 10. RXRG is the target of Bexarotene. Bexarotene treatment on HCT116 caused RXRG cluster formation and nucleolar translocation, indicating RXRG is the target of Bexarotene.

FIG. 11. Lentivirus-mediated RB1-KD caused dissociation of the complex and Emi1 cytoplasmic translocation in HCT116 on day 5.

FIGS. 12-18 depict exemplary compounds.

FIG. 19. Lentivirus-mediated RXRG KD kills RB1+neuroblastoma IMR32 cells and colon cancer HCT116 cells.

FIG. 20. RXRG KD cause cell cycle arrest at G1 phase in KRAS mutant colon cancer cell line HCT116

FIG. 21. A. RXRG mRNA level after RXRG KD in colon cancer HCT116. B & C: RXRG KD caused Rb dephosphorylation, PP2A phosphorylation and inactivation, Emi1 hyperphosphorylation and inactivation, and SKP2 downregulation, resulting in p27 and p21 accumulation in HCT116.

FIG. 22. RXRG antagonist HX531 treatment on cell lines (6 uM). EGFR, KRAS, and NRAS activated NSCLC, pancreatic, and colon cancers are sensitive to RXR antagonist HX531 treatment, but RB1 mutated retinoblastoma and Saos2, and normal fibroblasts WI38 are not sensitive to HX531 treatment. PTEN mutant prostate cancer line LnCap, SCLC line H446, and breast cancer cell line MDA-MB-468 are also sensitive to HX531 treatment.

FIG. 23. Relative cell number after RXRG antagonist UVI3003 treatment (10 uM). EGFR and KRAS activated NSCLC, pancreatic, gastric cancer, and colon cancers are sensitive to RXR antagonist UVI3003 treatment. Some PTEN mutated breast cancer MDA-MB-468 and SCLC, and some BRAF mutated tumor such as H1755 are also sensitive to UVI3003 treatment. Retinoblastoma, Saos2, and normal fibroblasts WI38 are not sensitive to UVI3003 treatment.

FIG. 24. Dose-dependent effect of HX531 on Colon cancer cell HCT116. A. RXRG antagonist HX531 suppresses KRAS mutant colon cancer cell growth. B. RXRG agonist Bexarotene slightly promotes KRAS mutant colon cancer cell growth at low dosages.

FIG. 25. Dose-dependent effect of HX531 on colon cancer cell CCCL-18. A. RXRG antagonist HX531 suppresses KRAS mutant colon cancer CCCL-18 cell growth. B. RXRG agonist Bexarotene promotes KRAS mutant colon cancer cell CCCL-18 growth at low dosages.

FIG. 26. KRAS mutant Non small cell lung cancer (NSCLC) A549 treated with RXRG ligands. KRAS mutant NSCLC cell A549 is sensitive to RXRG antagonists HX531 treatment, but resistant to agonist Bexarotene (B) treatment. d2 (A) and d3 (B).

FIG. 27. EGFR mutated Lung cancer cells such as H1975 (A), H3255 (B), H1650 (C), and H820 (D) are sensitive to RXR antagonists HX531, UVI3003, and PA452.

FIG. 28. KRAS mutated pancreatic cancer cells such as PC941102 (A), PC931019 (B), and PC930201 (C) are sensitive to RXR antagonists HX531, UVI3003, and PA452, but not bexarotene.

FIG. 29. Cell cycle synchronization and Cell cycle analysis showed G1 arrest after HX531 treatment in lung cancer A549 cells for 2 days. A: G1; B: S; C: G2/M.

FIG. 30. Cell cycle analysis showed HX531 (A) and UVI3003 (B) inhibited G1-S transition in EGFR mutant NSCLC cancer cell H1975 on day 3.

FIG. 31. Cell cycle analysis showed HX531 (A) and UVI3003 (B) inhibited G1-S transition in KRAS mutant pancreatic cancer cell PC931019 on day 3.

FIG. 32. Synergistic effects between HX531 and MEK inhibitors PD98059 and PD0325901 in colon cancer HCT116 on day 3. 6 uM HX531 caused significant growth inhibition in HCT116 (A) and A549 (B). MEK inhibitors (10 uM PD98059 and 100 nM PD0325901) caused moderate growth inhibition in HCT116 and A549. There were. Synergistic effects between HX531 and MEK inhibitors in HCT116.

FIG. 33. Synergistic effects between HX531 and MEK inhibitors in pancreatic cancer PC931019 on day 9. 6 uM HX531 caused growth inhibition in PC931019. MEK inhibitors (10 uM PD98059 and 100 nM PD0325901) caused growth inhibition in PC931019. There were Synergestic effects between HX531 and MEK inhibitors in PC931019.

FIG. 34. Western blot showed HX531 caused significant Emi1 downregulation and inactivation of Emi1, and degradation of SKP2 in HCT116 on day 2.

FIG. 35. Western blot showed that HX531, but not Bexarotene, dephosphorylated Rb and downregulated CDK2, Emi1, and SKP2 in HCT116 on day 2.

FIG. 36. RXRG antagonist dissociates S-phase promoting complex. RXRG antagonist HX531 dissociates pRb-S807 and TRB2 in colon cancer cell line HCT116.

FIG. 37. RXRG antagonist dissociates S-phase promoting complex. RXRG antagonist HX531 dissociates pRb-S807 and RXRG in colon cancer cell line HCT116.

FIG. 38. RXRG antagonist dissociates S-phase promoting complex. RXRG antagonist HX531 dissociates pRb-S807 and Emi1 in colon cancer cell line HCT116.

FIG. 39. RXRG antagonist dissociates S-phase promoting complex. RXRG antagonist HX531 dissociates pRb-S807 and p-p107 in colon cancer cell line HCT116.

FIG. 40. RXRG antagonist dissociates S-phase promoting complex RXRG antagonist HX531 dissociates pRb-S807 and pp130 in colon cancer cell line HCT116.

FIG. 41. RXRG antagonist dissociates S-phase promoting complex. RXRG antagonist HX531 dissociates CDK2 and Cyclin E in colon cancer cell line HCT116.

FIG. 42. HX531 treatment caused Cdh1 nuclear translocation in lung cancer cells A549.

FIG. 43. RXRG agonist bexarotene promoted S-phase promoting complex formation and phospho-Rb-RXRG interaction in HCT116.

FIG. 44. Targeting RXRG by HX531 causes proliferating complex dissociation and cell cycle arrest.

FIG. 45. HX531 (6 uM) treatment on A549 caused DNA condensation and separation defects (‘DNA thread’ formation, upper panel, arrow) demonstrated by DAPI staining on day 1. HX531 treatment significantly suppressed mitosis, which is common in control (lower panel, arrow head).

FIG. 46. HX531 treatment on A549 caused DNA condensation and separation defects (‘DNA thread’ formation) demonstrated by DAPI staining on day 1. HX531 treatment significantly reduced mitosis (DNA condensation). (Concentration of compounds: 6 uM HX531, uM Bexarotene, 100 nM PD0325901, 10 uM PD98059, 100 nM TPA, and 5 uM Nutlin 3A.

FIG. 47. HX531 treatment caused MDM2 downregulation and inactivation, p53 phosphorylation and activation in lung cancer A549 on day 2.

FIG. 48. mRNA Levels after treatment of RXR ligands. HX531 treatment on A549 cells and HCT116 caused p53 targeted genes (GADD45, HDM2, p27, and p21) upregulation, leading to apoptosis; whereas Bexarotene treatment caused downregulation of p53 targeted genes on day 2.

FIG. 49. HX531 activates p53 in A549 on day 2.

FIG. 50. Preliminary results showed that HX531 treatment could suppress lung cancer formation after tail vein injection of A549 NSCLC cells in nude mice. Two months after tail vein injection of one million A549 cells, lung cancer nodules could be detected on lung surface in control group, but not in HX531 treated group (100 ug/ml in drinking water). No significant side effects were detected after 2 months treatment of HX531.

FIG. 51. A: Targeting Synthetic Lethal interactions in KRAS or EGFR mutated cancers by RXRG antagonists; B: Simplified Rb-RXRG-THRB2-SKP2 pathway for cell cycle control and its targeted therapy for KRAS, EGFR or PTEN mutated cancers. KRAS, EGFR or PTEN mutated cancers such as colon, pancreatic, lung, breast, prostate cancers, and glioma etc can be treated by RXRG antagonists.

FIG. 52. Bexarotene treatment suppressed growth of BRAF mutant melanoma cell M21 and OCM1, PIK3CA mutant breast cancer MCF7, NSCLC lines H3122 and H2228 with EML4-ALK fusion, cervical cancer cell HELA with HPV infection, PTEN mutated prostate cancer, small cell lung cancer, and breast cancer, RB1 mutated osteosarcoma cell SAOS2, RB1 mutated prostate cancer DU145, some RB1 mutant SCLC H1417 and H209, and some neuroblastoma SKN-BE(2). Bexarotene did not have good effects on KRAS mutated HCT116, NRAS mutated large cell lung cancer H1299, and RB1 wild type U2OS.

FIG. 53. LKO lentivirus-mediated RXRG knockdown in Y79 (A) and RB176 (B) suppressed G1/S transition demonstrated by PI staining and cell cycle

FIG. 54. RXRG ligands on Retinoblastoma. RXRG agonists Bexarotene and 9 cis RA suppressed retinoblastoma cell growth. RXRG antagonist HX531 promoted retinoblastoma cell growth at low dosages.

FIG. 55. Cell number changes after treatment of RXRG ligands on RB. RXRG agonist Bexarotene suppressed RB176 (A) and WERI (B) cell growth.

FIG. 56A-B. Cell cycle analysis after Bexarotene treatment with different dosages on RB177. Bexarotene treatment caused G2-M block and polyploidy in RB177. A: RB177-Bexarotene-d6; B: RB177-2.

FIG. 57. Cell cycle synchronization and cell cycle analysis in RB177 after treatment with 10 uM bexarotene and HX531 for 2 days. Bexarotene treatment caused delayed S phase progression and delayed G2/M transition. A: G1; B: S; C: G2/M.

FIG. 58. p53 immuno-fluorescence showed Bexarotene activated p53 in RB177 on day 2.

FIG. 59. Bexarotene promoted whereas HX531 suppressed expression of p53-targeted genes such as 14-3-3, GADD45, p21, and MDM2 in RB177 (p<0.05).

FIG. 60. Bexarotene suppressed RB growth in mice. RB177 was grafted in subretinal space and treated with bexarotene. Subconjunctival (A) and oral administration (B) of Bexarotene could significantly suppress retinoblastoma growth in subretinal grafted mouse animal model.

FIG. 61. Bexarotene suppressed RB177 growth in mice. Subconjunctival and oral taking of bexarotene can significantly suppress retinoblastoma RB177 growth in subretinal xenograft mouse model (*p<0.05; #p<0.01).

FIG. 62. Targeted therapy of retinoblastoma by RXRG agonists. RXRG agonists such as Bexarotene promote TRB2 activity and G1-S transition, but they cause G2-M block, resulting in cell cycle arrest in retinoblastoma cells. G2-M block and stabilized securin caused DNA damage and apoptosis.

### DETAILED DESCRIPTION OF CERTAIN EMBODIMENTS

### 1. Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon, bicyclic hydrocarbon, or tricyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-30 aliphatic carbon atoms. In some embodiments, aliphatic groups contain 1-20 aliphatic carbon atoms. In other embodiments, aliphatic groups contain 1-10 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “cycloaliphatic,” as used herein, refers to saturated or partially unsaturated cyclic aliphatic monocyclic, bicyclic, or polycyclic ring systems, as described herein, having from 3 to 14 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic,” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, that has a single point of attachment to the rest of the molecule, or a C₉-C₁₆ tricyclic hydrocarbon that is completely saturated or that contains one or more units of unsaturation, but which is not aromatic, that has a single point of attachment to the rest of the molecule.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

The term “heteroalkyl” is given its ordinary meaning in the art and refers to alkyl groups as described herein in which one or more carbon atoms is replaced with a heteroatom (e.g., oxygen, nitrogen, sulfur, and the like). Examples of heteroalkyl groups include, but are not limited to, alkoxy, poly(ethylene glycol)-, alkyl-substituted amino, tetrahydrofuranyl, piperidinyl, morpholinyl, etc.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, tricyclic, tetracyclic, and/or otherwise polycyclic The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic; tricyclic, tetracyclic, and/or otherwise polycyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “halogen” means F, Cl, Br, or I.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —(CH₂)₀₋₄SR^{o}; —(CH₂)₀₋₄S(O)R^{o}; —(CH₂)₀₋₄S(O)₂R^{o}; —O(CH₂)₀₋₄R^{o}, —O—(CH₂)₀₋₄C(O)OR^{o}; —(CH₂)₀₋₄CH(OR^{o})₂; —(CH₂)₀₋₄Ph, which may be substituted with R^{o}; —(CH₂)₀₋₄(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂; wherein each R^{o} may be substituted as defined below and is independently hydrogen, C₁₋₆aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R., -(haloR.), —(CH₂)₀₋₂OH, —(CH₂)₀₋₂OR., —(CH₂)₀₋₂CH(OR.)₂; —O(haloR.), —CN, —N₃, —(CH₂)₀₋₂C(O)R., —(CH₂)₀₋₂C(O)OH, —(CH₂)₀₋₂C(O)OR., —(CH₂)₀₋₂SR., —(CH₂)₀₋₂SH, —(CH₂)₀₋₂NH₂, —(CH₂)₀₋₂NHR., —(CH₂)₀₋₂NR.₂, —NO₂, —SiR.₃, —OsiR.₃, —C(O)SR., —(C₁₋₄ straight or branched alkylene)C(O)OR., or —SSR. wherein each R. is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR^{*}₂, ═NNHC(O)R*, ═NNHC(O)OR*, ═NNHS(O)₂R*, ═NR*, ═NOR*, —O(C(R^{*}₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R., -(haloR.), —OH, —OR., —O(haloR.), —CN, —C(O)OH, —C(O)OR., —NH₂, —NHR., —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R., -(haloR.), —OH, —OR., —O(haloR.), —CN, —C(O)OH, —C(O)OR., —NH₂, —NHR., —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The phrase “protecting group,” as used herein, refers to temporary substituents which protect a potentially reactive functional group from undesired chemical transformations. Examples of such protecting groups include esters of carboxylic acids, silyl ethers of alcohols; and acetals and ketals of aldehydes and ketones, respectively. A “Si protecting group” is a protecting group comprising a Si atom, such as Si-trialkyl (e.g., trimethylsilyl, tributylsilyl, t-butyldimethylsilyl), Si-triaryl, Si-alkyl-diphenyl (e.g., t-butyldiphenylsilyl), or Si-aryl-dialkyl (e.g., Si-phenyldialkyl). Generally, a Si protecting group is attached to an oxygen atom. The field of protecting group chemistry has been reviewed (Greene, T. W.; Wuts, P. G. M. Protective Groups in Organic Synthesis, 2nd ed.; Wiley: New York, 1991). Such protecting groups (and associated protected moieties) are described in detail below.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention. Such compounds are useful, for example, as analytical tools or probes in biological assays.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, use of the term “about” in reference to dosages means±5 mg/kg/day.

Characteristic portion: As used herein, the phrase a “characteristic portion” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, 10, 15, 20 or more amino acids are required to be characteristic of a protein. In general, a characteristic portion is one that, in addition to the sequence identity specified above, shares at least one functional characteristic with the relevant intact protein.

Intraperitoneal: The phrases “intraperitoneal administration” and “administered intraperitonealy” as used herein have their art-understood meaning referring to administration of a compound or composition into the peritoneum of a subject.

In vitro: As used herein, the term “in vitro” refers to events that occur in an artificial environment, e.g., in a test tube or reaction vessel, in cell culture, etc., rather than within an organism (e.g., animal, plant, and/or microbe).

In vivo: As used herein, the term “in vivo” refers to events that occur within an organism (e.g., animal, plant, and/or microbe).

Oral: The phrases' “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Patient: As used herein, the term “patient”, “subject”, or “test subject” refers to any organism to which butaclamol is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.). In some embodiments, a subject may be suffering from, and/or susceptible to a disease, disorder, and/or condition (e.g., a neurodegenerative disease, a disease, disorder or condition associated with protein aggregation, ALS, etc.).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Prodrug: A general, a “prodrug”, as that term is used herein and as is understood in the art, is an entity that, when administered to an organism, is metabolized in the body to deliver a therapeutic agent of interest. Various forms of “prodrugs” are known in the art. For examples of such prodrug derivatives, see:
a) Design of Prodrugs, edited by H. Bundgaard, (Elsevier, 1985) and Methods in Enzymology, 42:309-396, edited by K. Widder, et al. (Academic Press, 1985);
b) A Textbook of Drug Design and Development, edited by Krogsgaard-Larsen;
c) Bundgaard, Chapter 5 “Design and Application of Prodrugs”, by H. Bundgaard, p. 113-191 (1991);
d) Bundgaard, Advanced Drug Delivery Reviews, 8:1-38 (1992);
e) Bundgaard, et al., Journal of Pharmaceutical Sciences, 77:285 (1988), and
f) Kakeya, et al., Chem. Pharm. Bull., 32:692 (1984).

Protein: As used herein, the term “protein” refers to a polypeptide (i.e., a string of at least two amino acids linked to one another by peptide bonds). In some embodiments, proteins include only naturally-occurring amino acids. In some embodiments, proteins include one or more non-naturally-occurring amino acids (e.g., moieties that form one or more peptide bonds with adjacent amino acids). In some embodiments, one or more residues in a protein chain contains a non-amino-acid moiety (e.g., a glycan, etc). In some embodiments, a protein includes more than one polypeptide chain, for example linked by one or more disulfide bonds or associated by other means. In some embodiments, proteins contain L-amino acids, D-amino acids, or both; in some embodiments, proteins contain one or more amino acid modifications or analogs known in the art. Useful modifications include, e.g., terminal acetylation, amidation, methylation, etc. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

For purposes of this invention, the chemical elements are identified in accordance with the Periodic Table of the Elements, CAS version, Handbook of Chemistry and Physics, 67th Ed., 1986-87, inside cover.

Several methods/assays can be used to determine if a compound is an RXRG agonist or antagonist. As illustrated in “MDMX and MDM2 promoter luciferase assay” in the Exemplification section, one method well known to a person of ordinary skill in the art is based on an MDMX and MDM2 promoter luciferase assay, in which an RXRG agonists promotes MDMX and MDM2 promoter luciferase activity, whereas an RXRG antagonist suppresses MDMX and MDM2 promoter luciferase activity. Other methods include a HL-60 proliferation assay and retinoblastoma tests (wherein an antagonist promotes HL-60 or retinoblastoma growth, and an agonist suppresses HL-60 or retinoblastoma growth), which are known to the skilled artisan. It will be appreciated that the determination of whether a compound is a RXRG agonist or RXRG antagonist can be based on the outcome of one or more of these assays described in the Exemplification section.

### 2. Description of Certain Embodiments of the Invention

The present invention encompasses the recognition that there exists a need for methods for treating patients suffering from or susceptible to cancer. The present invention provides, among other things, methods of using retinoid X receptor gamma agonists and antagonists to treat cancer.

The present application specifically describes, for the first time, a proliferating nuclear complex (Treprec-Xu), comprising thyroid hormone receptor beta2 (TRB2), Emi1, phosphorylated Rb family proteins (i.e., one or more of RB, p130, and p107), cyclin E, CDK2, and retinoid X receptor gamma (RXRG). Among other things, the present invention provides strategies for identifying cancer therapies that alter level and/or activity of the Treprec-Xu complex, for example by altering (e.g., inhibiting or promoting) association of one or more components of the complex with each other and/or with the complex generally. In some embodiments, the present invention provides strategies for identifying agents that alter association of RXRG with and/or activity of RXRG within the complex. The present invention also provides methods of treating cancer with such agents.

The present invention provides methods for inhibiting association of the Treprec-Xu complex. In some embodiments, inhibition of Treprec-Xu association is characterized by inhibiting RXRG association with other members of the Treprec-Xu complex. Through inhibition of the Treprec-Xu complex, the present invention provides methods useful in the treatment of many human cancers. In certain embodiments, the protein interactions targeted by methods of the present invention are selected from the group consisting of Phospho-Rb and Emi1, Phospho-Rb and TRB2, Phospho-Rb and RXRG, RXRG and TRB2, TRB2 and Emi1, TRB2 and pp107, TRB2 and cyclin E.

The present invention provides evidence that the Treprec-Xu components need to associate together for maintenance of Emi1 phosphorylation and activation. Emi1 can suppress tumor suppressor APC/Cdh1 and push cells through interphase. The Treprec-Xu complex is involved in the G1-S cell cycle transition and is an important player in cell proliferation and tumorigenesis. While not wishing to be bound by any particular theory, it appears that TRB1 can counteract TRB2 by recruiting PP1 to the Treprec-Xu complex, thereby causing dephosphorylation of Rb and dissociation of complex. Phosphorylated Rb promotes RXRG binding to TRB2 and maintains the complex.

The present disclosure recognizes that Rb plays an important role in colon cancer tumorigenesis. RB1 knockdowns in colon cancer cell HCT116 significantly kills colon cancer. Applicant has identified an important proliferating nuclear complex in proliferating cancer cells.

In some embodiments, a Treprec-Xu complex comprises all three phosphorylated Rb family proteins selected from the group consisting of Rb, p107, and p130. In some embodiments, a Treprec-Xu complex comprises two phosphorylated Rb family proteins selected from the group consisting of Rb, p107, and p130. In some embodiments, a Treprec-Xu complex comprises one phosphorylated Rb family protein selected from the group consisting of Rb, p107, and p130. While not wishing to be bound by any particular theory, it appears that p-Rb and p-p107 promote Treprec-Xu complex formation whereas p130 promotes dissociation. In some embodiments, TRB2 and p-p107 can replace p-Rb to maintain the complex if an RB1 mutation is present.

Knockdowns of RB1 and RXRG cause cell cycle arrest in G1-S phase by APC/cdh1 mediated ubiquitination and degradation of SKP2, an important oncogene for cell cycle progression. These results indicate that RB1 and RXRG are synthetic lethal genes in colon cancer after K-Ras mutagenetic activation. The present disclosure recognizes that targeting Rb-RXRG-TRB2-Emi1 interaction is a useful strategy for cancer treatment.

In fact, Applicant has found that RXRG antagonists can efficiently kill KRAS or EGFR mutated colon cancer, pancreatic cancer, non-small cell lung cancer (NSCLC), and heptoma cells growth. In certain embodiments, the RXRG antagonist HX531 can significantly suppress colon cancer and non-small cell lung cancer proliferation and cause cell cycle arrest by dissociation of the Treprec-Xu complex. This effect was not observed in normal fibroblast cell WI38. In certain embodiments, RXRG antagonists are useful for the treatment of PTEN mutated cancers such as prostate cancer, breast cancer, glioma, and some melanoma. In some embodiments, RXRG antagonists are useful for the treatment of MEK-ERK-activated cancers such as colorectal cancer, NSCLC, gastric cancer, pancreatic cancer, hepatoma, breast cancer, myeloid leukemia, some neuroblastoma, thyroid cancer, and prostate cancer, to name but a few. In some embodiments, MEK-ERK-activated tumors include, but are not limited to, those having a RAS activated mutation, RAF activated mutation, EGFR/HER2 activation, PDGFR activation, NF1 inactivation, or ERG and ETV activation.

In some embodiments, the present invention observes that particular RXRG antagonists are surprisingly effective at inhibiting proliferation of certain cancer types. For example, the present invention specifically demonstrates the activity of compound HX531 in the inhibition of certain cancers, for example but not limiting to, KRAS mutant colon cancers (HCT116, CCCL13, CCCL18), NSCLC (A549, H460, H2030, H358), pancreatic cancers (PC1102, PC1019, PC0201), EGFR mutant NSCLCs (H1975, H1650, H820, and H3255), PTEN mutant breast cancer MDA-MD-468, prostate cancers LnCap and PC3, and SCLC H446 cancer cells. Other cell lines in which growth inhibition is observed with compound HX531 include M21 (BRAF mutated melanoma), C918 (melanoma), H1755 (BRAF mutated NSCLC), HepG2 (hepatoma), IMR32 (neuroblastoma), NCI-H1299 (NRAS mutant Large cell lung cancer), DU145 (prostate cancer), MDA-MB-453 (HER2 positive breast cancer), U2OS (osteosarcoma), and H209 (SCLC) cells.

Other RXRG antagonists such as UVI3003 and PA452 have also been demonstrated to efficiently suppress KRAS or EGFR mutant colorectal cancer, non-small cell lung cancer, pancreatic cancer, and heptoma cells. These RXRG antagonists also suppress the growth of PTEN mutant prostate cancer and breast cancer, and some BRAF mutant NSCLC and melanoma.

In other embodiments, the present invention observes that particular RXRG agonists are surprisingly effective at inhibiting proliferation of certain cancer types without KRAS and EGFR mutation. For example, the present invention specifically demonstrates surprising activity of bexarotene, a RXRG agonist, in the inhibition of RB1 mutant retinoblastoma growth both in vitro and in in vivo animal xenograft model. In some embodiments, bexarotene significantly suppresses BRAF mutant melanoma cells M21 and OCM1, PIK3CA mutant negative MCF7, NSCLC H3122 and H2228 with EML4-ALK fusion, cervical cancer cell HELA with HPV infection, RB1 mutated osteosarcoma cell SAOS2, RB1 mutated prostate cancer DU145, PTEN mutant breast cancer, prostate cancer, SCLC, and glioma, certain RB1 mutant SCLC, and certain neuroblastoma cells SKN-BE(2).

As described above, methods of the present invention are useful in the treatment of cancer. In certain embodiments, methods of the present invention may be used in the treatment or prevention of neoplasms. In certain embodiments, the neoplasm is a benign neoplasm. In other embodiments, the neoplasm is a malignant neoplasm. In certain embodiments, the cancer is a solid tumor. Exemplary cancers that may be treated using inventive compounds include those described above and herein. In some embodiments, the cancer originates from any one of the above-mentioned organs or tissues

In some embodiments, the invention provides methods of treating a subject suffering from or susceptible to cancer with KRAS, EGFR or PTEN mutations with a therapeutically effective amount of a RXRG antagonist.

In one aspect, the invention provides methods of inhibiting growth of cancer cells with KRAS, EGFR or PTEN mutations with a retinoid X receptor gamma (RXRG) antagonist.

In one aspect, the invention provides methods of inhibiting proliferation of cancer cells with KRAS, EGFR or PTEN mutations, with a retinoid X receptor gamma (RXRG) antagonist.

In one aspect, the invention provides methods of promoting apoptosis of cancer cells with KRAS, EGFR or PTEN mutations with a retinoid X receptor gamma (RXRG) antagonist.

In one aspect, the invention provides methods of suppressing G1/S transition in cancer cells with KRAS, EGFR or PTEN mutations with a retinoid X receptor gamma (RXRG) antagonist.

In one aspect, the invention provides methods of treating a subject suffering from or susceptible to a cancer without KRAS, EGFR or PTEN mutations with a retinoid X receptor gamma (RXRG) agonist. In one aspect, the invention provides methods of treating a subject suffering from or susceptible to a cancer without KRAS, or EGFR mutations with a retinoid X receptor gamma (RXRG) agonist. In one aspect, the invention provides methods of treating a subject suffering from or susceptible to a cancer without KRAS, or EGFR mutations and with RB1, BRAF, P1K3CA, PTEN, or EML4-ALK mutation or HPV infection, with a retinoid X receptor gamma (RXRG) agonist.

In one aspect, the invention provides methods of inhibiting growth of cancer cells without KRAS, EGFR or PTEN mutations with a retinoid X receptor gamma (RXRG) agonist. In one aspect, the invention provides methods of inhibiting growth of cancer cells without KRAS or EGFR mutations, with a retinoid X receptor gamma (RXRG) agonist. In one aspect, the invention provides methods of inhibiting growth of cancer cells without KRAS or EGFR mutations and with RB1, BRAF, PIK3CA, PTEN, or EML4-ALK mutation or HPV infection, with a retinoid X receptor gamma (RXRG) agonist.

In one aspect, the invention provides methods of inhibiting proliferation of cancer cells without KRAS, EGFR or PTEN mutations with a retinoid X receptor gamma (RXRG) agonist. In one aspect, the invention provides methods of inhibiting proliferation of cancer cells without KRAS or EGFR mutations, with a retinoid X receptor gamma (RXRG) agonist. In one aspect, the invention provides methods of inhibiting proliferation of cancer cells without KRAS or EGFR mutations and with RB1, BRAF, PIK3CA, PTEN, or EML4-ALK mutation or HPV infection, with a retinoid X receptor gamma (RXRG) agonist.

In one aspect, the invention provides methods of promoting apoptosis of cancer cells without KRAS, EGFR or PTEN mutations with a retinoid X receptor gamma (RXRG) agonist. In one aspect, the invention provides methods of promoting apoptosis of cancer cells without KRAS or EGFR mutations, with a retinoid X receptor gamma (RXRG) agonist. In one aspect, the invention provides methods of promoting apoptosis of cancer cells without KRAS or EGFR mutations and with RB1, BRAF, PIK3CA, PTEN, or EML4-ALK mutation or HPV infection, with a retinoid X receptor gamma (RXRG) agonist.

In one aspect, the invention provides methods of suppressing G1/S transition in cancer cells without KRAS, EGFR or PTEN mutations with a retinoid X receptor gamma (RXRG) agonist. In one aspect, the invention provides methods of suppressing G1/S transition in cancer cells without KRAS or EGFR mutations, with a retinoid X receptor gamma (RXRG) agonist. In one aspect, the invention provides methods of suppressing G1/S transition in cancer cells without KRAS or EGFR mutations and with RB1, BRAF, PIK3CA, PTEN, or EML4-ALK mutation or HPV infection, with a retinoid X receptor gamma (RXRG) agonist.

In one aspect, the invention provides methods of modulating functions of Treprec-Xu complex in cancer. In one aspect, the invention provides methods of modulating functions of Treprec-Xu complex in cancer by inhibiting or promoting association or dissociation of one or more components of the complex with each other and/or with the complex.

The present invention relates to the function of retinoid X receptor gamma (RXRG) in cancer. In one aspect, the present invention provides methods to modulate RXRG function in cancer.

In some embodiments, the present invention provides methods comprising the step of administering to a subject suffering from or susceptible to cancer a therapeutically effective amount of a compound of formula I:

wherein,
R¹ is hydrogen or an optionally substituted C₁₋₁₂ aliphatic group;
each R² is independently halogen, R′, —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —N(R)C(O)N(R)₂, —N(R)SO₂N(R)₂, —N(R)SO₂R, —OC(O)N(R)₂, or an optionally substituted C₁₋₁₂ aliphatic group, or two R² groups on adjacent carbon atoms are taken together with their intervening atoms to form an optionally substituted 5- to 7-membered ring having 0-4 heteroatoms selected from nitrogen, oxygen, or sulfur;
each R³ is independently halogen, R′, —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —OSO₂R, —N(R)C(O)N(R)₂, —N(R)SO₂N(R)₂, —N(R)SO₂R, —OC(O)N(R)₂, or an optionally substituted C₁₋₁₂ aliphatic group, or two R³ groups on adjacent carbon atoms are taken together with their intervening atoms to form an optionally substituted 5- to 7-membered ring having 0-4 heteroatoms selected from nitrogen, oxygen, or sulfur;
m is from 0 to 4, inclusive;
p is from 0 to 4, inclusive;
T is a covalent bond or an optionally substituted, bivalent C₁₋₆ saturated or unsaturated, straight or branched, hydrocarbon chain, wherein one or two methylene units of T are optionally and independently replaced by —Cy-, —C(R)₂—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Cy is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
X is a covalent bond, —O—, —NR—, —NR³—, —NCH₂R³—, —C(R)₂—, —C(═CH₂)—, —CHR³—, —C(R³)₂—, or —S—;
each R is independently hydrogen or R′;
each R′ is independently an optionally substituted group selected from C₁₋₁₀ aliphatic, phenyl, a 3-7 membered saturated or partially unsaturated carbocyclic ring, a 3-7 membered saturated or partially unsaturated monocyclic heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur; or:
   two R′ groups on the same nitrogen are taken together with their intervening atoms to form an optionally substituted 3-7 membered saturated, partially unsaturated, or heteroaryl ring having 1-4 heteroatoms independently selected from nitrogen, oxygen, or sulfur.

In some embodiments, R¹ is H. In some embodiments, R¹ is optionally substituted C₁₋₆ aliphatic. In some embodiments, R¹ is methyl.

In some embodiments, at least one R² is halogen. In some embodiments, at least one R² is R′, wherein each R′ is independently as defined above and described herein. In some embodiments, at least one R² is optionally substituted C₁₋₁₀ aliphatic. In some embodiments, at least one R² is optionally substituted C₁₋₁₀ alkyl. In some embodiments, at least one R² is methyl.

In some embodiments, at least one R² is —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —N(R)C(O)N(R)₂, —N(R)SO₂N(R)₂, —N(R)SO₂R, —OC(O)N(R)₂, or an optionally substituted C₁₋₁₂ aliphatic group, or two R² groups on adjacent carbon atoms are taken together with their intervening atoms to form an optionally substituted 5- to 7-membered ring having 0-4 heteroatoms selected from nitrogen, oxygen, or sulfur; wherein each R is independently as defined above and described herein.

In some embodiments, at least one R² is —OR, wherein each R is independently as defined above and described herein. In some embodiments, at least one R² is —OH. In some embodiments, at least one R² is —OR, wherein each R is independently an optionally substituted C₁₋₁₀ aliphatic. In some embodiments, at least one R² is —OR, wherein each R is independently an optionally substituted C₁₋₁₀ alkyl. In some embodiments, at least one R² is —OR, wherein R is n-octyl. In some embodiments, at least one R² is —OR, wherein R is n-heptyl. In some embodiments, at least one R² is —OR, wherein R is n-hexyl. In some embodiments, at least one R² is —OR, wherein R is n-pentyl. In some embodiments, at least one R² is —OR, wherein R n-butyl. In some embodiments, at least one R² is —OR, wherein R n-propyl. In some embodiments, at least one R² is —OR, wherein R ethyl. In some embodiments, at least one R² is —OR, wherein R n-methyl. In some embodiments, R² is —CH═CH—COOH.

In some embodiments, at least one R² is —OR, wherein each R is independently an optionally substituted phenyl. In some embodiments, at least one R² is —OR, wherein each R is independently phenyl. In some embodiments, at least one R² is —OR, wherein each R is independently 4-methylphenyl. In some embodiments, at least one R² is —OR, wherein each R is independently 4-trifluoromethylphenyl. In some embodiments, at least one R² is —OR, wherein each R is independently naphthyl. In some embodiments, at least one R² is —OR, wherein each R is independently naphthyl.

In some embodiments, at least one R² is —NO₂. In some embodiments, at least one R² is —COOH.

In some embodiments, two R² groups on adjacent carbon atoms are taken together with their intervening atoms to form an optionally substituted 6-membered carbocyclic ring. In some embodiments, two R² groups on adjacent carbon atoms are taken together with their intervening atoms to form a 6-membered carbocyclic ring substituted with four methyl groups.

In some embodiments, at least one R³ is halogen. In some embodiments, at least one R³ is R′, wherein each R′ is independently as defined above and described herein. In some embodiments, at least one R³ is optionally substituted C₁₋₁₀ aliphatic. In some embodiments, at least one R³ is optionally substituted C₁₋₁₀ alkyl. In some embodiments, at least one R³ is methyl.

In some embodiments, at least one R³ is —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —OSO₂R, —N(R)C(O)N(R)₂, —N(R)SO₂N(R)₂, —N(R)SO₂R, —OC(O)N(R)₂, or an optionally substituted C₁₋₁₂ aliphatic group, or two R³ groups on adjacent carbon atoms are taken together with their intervening atoms to form an optionally substituted 5- to 7-membered ring having 0-4 heteroatoms selected from nitrogen, oxygen, or sulfur; wherein each R is independently as defined above and described herein.

In some embodiments, at least one R³ is —OR, wherein each R is independently as defined above and described herein. In some embodiments, at least one R³ is —OH. In some embodiments, at least one R³ is —OR, wherein each R is independently an optionally substituted C₁₋₁₀ aliphatic. In some embodiments, at least one R³ is —OR, wherein each R is independently an optionally substituted C₁₋₁₀ alkyl. In some embodiments, at least one R³ is —OR, wherein R is n-octyl. In some embodiments, at least one R³ is —OR, wherein R is n-heptyl. In some embodiments; at least one R³ is —OR, wherein R is n-hexyl. In some embodiments, at least one R³ is —OR, wherein R is n-pentyl. In some embodiments, at least one R³ is —OR, wherein R n-butyl. In some embodiments, at least one R³ is —OR, wherein R n-propyl. In some embodiments, at least one R³ is —OR, wherein R n-ethyl. In some embodiments, at least one R³ is —OR, wherein R methyl. In some embodiments, R³ is —CH═CH—COOH.

In some embodiments, at least one R³ is —OR, wherein each R is independently an optionally substituted phenyl. In some embodiments, at least one R³ is —OR, wherein each R is independently phenyl. In some embodiments, at least one R³ is —OR, wherein each R is independently 4-methylphenyl. In some embodiments, at least one R³ is —OR, wherein each R is independently 4-trifluoromethylphenyl. In some embodiments, at least one R³ is —OR, wherein each R is independently naphthyl. In some embodiments, at least one R³ is —OR, wherein each R is independently naphthyl.

In some embodiments, at least one R³ is —NO₂. In some embodiments, at least one R³ is —COOH.

In some embodiments, two R³ groups on adjacent carbon atoms are taken together with their intervening atoms to form an optionally substituted 6-membered carbocyclic ring. In some embodiments, two R³ groups on adjacent carbon atoms are taken together with their intervening atoms to form a 6-membered carbocyclic ring substituted with four methyl groups.

As generally defined above, m is from 0 to 4, inclusive. In some embodiments, m is 0. In some embodiments, m is 1. In some embodiments, m is 2. In some embodiments, m is 3. In some embodiments, m is 4.

As generally defined above, p is from 0 to 4, inclusive. In some embodiments, p is 0. In some embodiments, p is 1. In some embodiments, p is 2. In some embodiments, p is 3. In some embodiments, p is 4.

In some embodiments, T is —Cy-, and Cy is an optionally substituted bivalent phenyl ring. In some embodiments; T is —Cy-, and Cy is a substituted bivalent phenyl ring. In some embodiments, T is —Cy-, and Cy is an unsubstituted bivalent phenyl ring. In some embodiments, T is

In some embodiments, Cy is optionally substituted bivalent phenyl ring. In some embodiments, Cy is a substituted bivalent phenyl ring. In some embodiments, Cy is an unsubstituted bivalent phenyl ring. In some embodiments, Cy is

In some embodiments, X is —O—, —NR—, —C(R)₂—, or —S—; wherein each R is independently as defined above and described herein. In some embodiments, X is a covalent bond. In some embodiments, X is —O—. In some embodiments, X is —NR—, wherein R is as defined above and described herein. In some embodiments, X is —N(Me)-. In some embodiments, X is —N(cyclopropylmethyl)-. In some embodiments, X is —N(SO₂Me)-. In some embodiments, X is —C(R)₂—, wherein each R is independently as defined above and described herein. In some embodiments, X is —C(═CH₂)—. In some embodiments, X is —CHR³—, wherein R³ is as defined above and described herein. In some embodiments, X is —NR³—, wherein R³ is as defined above and described herein. In some embodiments, X is —NCH₂R³—, wherein R³ is as defined above and described herein. In some embodiments, X is —C(R³)₂—, wherein each R³ is independently as defined above and described herein. In some embodiments, X is S.

As generally defined above, each R is independently hydrogen or R′, wherein R′ is as defined above and described herein. In some embodiments, R is hydrogen. In some embodiments, R is R′, wherein each R′ is independently as defined above and described herein.

In some embodiments, R′ is independently optionally substituted C₁₋₁₀ aliphatic. In some embodiments, R′ is independently optionally substituted C₁₋₁₀ alkyl. In some embodiments, R′ is n-octyl. In some embodiments, R′ is n-heptyl. In some embodiments, R′ is n-hexyl. In some embodiments, R′ is n-pentyl. In some embodiments, R′ is n-butyl. In some embodiments, R′ is n-propyl. In some embodiments, R′ is ethyl. In some embodiments, R′ is methyl.

In some embodiments, R′ is independently optionally substituted phenyl. In some embodiments, R′ is phenyl. In some embodiments, R′ is 4-methylphenyl. In some embodiments, R′ is 4-trifluoromethylphenyl. In some embodiments, R′ is naphthyl.

In some embodiments, a compound of formula I is a compound of formula II, III, IV, or V:

wherein:

each R^{3a} is independently C₁₋₆ alkyl; and

k is from 0 to 6, inclusive.

In some embodiments, the methods of the present invention use a compound of formula VI, VII or VIII:

wherein each of R¹, R, X, R³, and p is as described above and each Y is independently ═CH— or ═N—.

In some embodiments, a compound of formula VI is a compound of formula VI-a, below:

wherein
R⁴ is R³, or an optionally substituted 8-10 membered saturated, partially unsaturated, or aryl bicyclic ring having 0-5 heteroatoms independently selected from nitrogen, oxygen, or sulfur; and
each of Y R, R¹ and R³ is independently as defined above and described herein.

In some embodiments, a compound of formula VI is a compound of formula VI-a, below:

wherein:

R³ is —OR or optionally substituted C₁₋₁₀ aliphatic;

R⁴ is selected from hydrogen, optionally substituted C₁₋₅ alkyl, CH₃CO—, —OR and —SO₂R; and

each of R and Y is independently as defined above and described herein.

In some embodiments, a compound of formula VI is a compound of formula VI-b, VI-c, VI-d, VI-e, or VI-f below:

wherein each of R³ and R⁴ is independently as defined above and described herein. In some embodiments, a compound of formula VI is a compound of formula VI-b, VI-c, VI-e, or VI-f below:

wherein each R³ is independently selected from n-heptyl, n-octyl, —CH₃, —C₂H₅, n-C₃H₇, n-C₄H₉, n-C₅H₁₁, n-C₆H₁₃, phenyl,

In some embodiments, a compound of formula VI is a compound selected from:

In some embodiments, a compound of formula VI is a compound of formula VI-g, VI-h, VI-i, or VI-j below:

wherein each R is independently selected from n-heptyl, n-octyl, —CH₃, —C₂H₅, n-C₃H₇, n-C₄H₉, n-C₅H₁₁, n-C₆H₁₃, phenyl,

In some embodiments, a compound of formula VI is a compound of formula VI-k, VI-l, VI-m, VI-n, or VI-o below:

wherein each R³ is independently selected from

or —OR, wherein R is selected from n-heptyl, n-octyl, —CH₃, —C₂H₅, n-C₃H₇, n-C₄H₉, n-C₅H₁₁, n-C₆H₁₃, phenyl,

In some embodiments, the methods of the present invention use a compound of formula XI:

wherein each of R¹, R², R, X, R³, m and p is independently as defined above and described herein.

In some embodiments, a compound of formula XI is a compound of formula XI-a, below:

wherein each of R and R² is independently as defined above and described herein.

In certain embodiments, a compound of formula I, II, III, IV, V, VI, VII, VIII, or XI is selected from those depicted in Table 1, below.

```
TABLE 1
  HX630 (2)
  CD3254 (3)
  4
  5a
  HX600 (6a)
  HX602 (6b)
  HX603 (6c)
  HX604 (6d)
  HX605 (6e)
  HX607 (6f)
  HX531
  HX533 (7b)
  HX535 (7c)
  HX539 (7d)
  HX541 (7e)
  HX543 (7f)
  HX560 (7g)
  HX710 (8a)
  HX711 (8b)
  HX741 (8c)
  HX743 (8d)
  HX745 (8e)
  UVI3003 (8)
  PA452 (16)
  17
  Bexarotene (LGD1069)
  PA024
```

Other compounds for use in accordance with provided methods include:

In some embodiments, methods of the present invention employ a pharmaceutical composition comprising a compound of formula I, II, III, IV, V, VI, VII, VIII, or XI, or any compound disclosed herein. Pharmaceutical compositions may further comprise other therapeutically active ingredients (e.g., chemotherapeutic and/or palliative). For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

Compounds may be combined with a pharmaceutically acceptable carrier to form a pharmaceutical composition. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. In certain embodiments, the pharmaceutical composition includes a pharmaceutically acceptable amount of an inventive compound. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, from about 5% to about 70%, or from about 10% to about 30%.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations useful with the present invention include those suitable for oral, nasal, topical (including buccal and sublingual), rectal, vaginal and/or parenteral administration. The formulations may conveniently be presented in unit dosage form and may be prepared by any methods well known in the art of pharmacy. In certain embodiments, a formulation comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations include the step of bringing into association a compound with the carrier and, optionally, one or more accessory ingredients. In general, the formulations are prepared by uniformly and intimately bringing into association a compound with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound may also be administered as a bolus, electuary or paste.

In solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such carriers as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions suitable for parenteral administration comprise one or more compounds in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

Drug-eluting forms include coated or medicated stents and implantable devices. Drug-eluting stents and other devices may be coated with a compound or pharmaceutical preparation and may further comprise a polymer designed for time-release.

In certain embodiments, a compound or pharmaceutical preparation is administered orally. In other embodiments, the compound or pharmaceutical preparation is administered intravenously. In certain embodiments, a compound is attached via a cleavable linker to a solid support that is administered with a catheter. Alternative routes of administration include sublingual, intramuscular, and transdermal administrations.

When the compounds are administered as pharmaceuticals, to humans and animals, they can be given per se or as a pharmaceutical composition containing, for example, 0.1% to 99.5%, or 0.5% to 90%, of active ingredient in combination with a pharmaceutically acceptable carrier.

The preparations may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

These compounds may be administered to humans and other animals for therapy by any suitable route of administration, including orally, nasally, as by, for example, an aerosol, a spray, rectally, intravaginally, parenterally, intracisternally and topically, as by powders, ointments or drops, including buccally and sublingually.

Regardless of the route of administration selected, the compounds, which may be used in a suitable hydrated form, and/or the pharmaceutical compositions, are formulated into pharmaceutically-acceptable dosage forms by conventional methods known to those of skill in the art.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds employed in the pharmaceutical composition at levels lower than that required to achieve the desired therapeutic effect and then gradually increasing the dosage until the desired effect is achieved.

In some embodiments, a compound or pharmaceutical composition is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition repeatedly over the life of the subject. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six, or more sub-doses administered separately at appropriate intervals throughout the day, optionally, in unit dosage forms.

While it is possible for a compound to be administered alone, it is preferable to administer the compound as a pharmaceutical formulation (composition) as described above.

The compounds according to invention may be formulated for administration in any convenient way for use in human or veterinary medicine, by analogy with other pharmaceuticals.

As described above, the invention provides, among other things, methods of treating a subject suffering from or susceptible to cancer with KRAS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to cancer with KRAS, EGFR or PTEN mutations with a retinoid X receptor gamma (RXRG) antagonist, wherein the antagonist is compound HX531.

In some embodiments, the invention provides methods of treating a subject suffering from or susceptible to colorectal cancer with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to colorectal cancer with KARS, EGFR or PTEN mutations with a retinoid X receptor gamma (RXRG) antagonist, wherein the antagonist is compound HX531.

In some embodiments, the invention provides methods of treating a subject suffering from or susceptible to colon cancer with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to colon cancer with KARS, EGFR or PTEN mutations with a retinoid X receptor gamma (RXRG) antagonist, wherein the antagonist is compound HX531.

In some embodiments, the invention provides methods of treating a subject suffering from or susceptible to pancreatic cancer with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to pancreatic cancer with KARS, EGFR or PTEN mutations with a retinoid X receptor gamma (RXRG) antagonist, wherein the antagonist is compound HX531.

In some embodiments, the invention provides methods of treating a subject suffering from or susceptible to lung cancer with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to lung cancer with KARS, EGFR or PTEN mutations with a retinoid X receptor gamma (RXRG) antagonist, wherein the antagonist is compound HX531.

In some embodiments, the invention provides methods of treating a subject suffering from or susceptible to non-small cell lung cancer with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to non-small cell lung cancer with KARS, EGFR or PTEN mutations with a retinoid X receptor gamma (RXRG) antagonist, wherein the antagonist is compound HX531.

In some embodiments, the invention provides methods of treating a subject suffering from or susceptible to gastric cancer with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to gastric cancer with KARS, EGFR or PTEN mutations with a retinoid X receptor gamma (RXRG) antagonist, wherein the antagonist is compound HX531.

In some embodiments, the invention provides methods of treating a subject suffering from or susceptible to breast cancer with KRAS, EGFR, HER2, or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to breast cancer with KRAS, EGFR, HER2, or PTEN mutations with a retinoid X receptor gamma (RXRG) antagonist, wherein the antagonist is compound HX531.

In some embodiments, the invention provides methods of treating a subject suffering from or susceptible to leukemia with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to leukemia with KARS, EGFR or PTEN mutations with a retinoid X receptor gamma (RXRG) antagonist, wherein the antagonist is compound HX531.

In some embodiments, the invention provides methods of treating a subject suffering from or susceptible to glioma with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to glioma with KARS, EGFR or PTEN mutations with a retinoid X receptor gamma (RXRG) antagonist, wherein the antagonist is compound HX531.

In some embodiments, the invention provides methods of treating a subject suffering from or susceptible to prostate cancer with KARS, EGFR or PTEN mutations with a retinoid X receptor gamma (RXRG) antagonist, wherein the antagonist is of formula I, II, III, IV, V, VI, VII, VIII, or XI. In some embodiments, the invention provides methods of treating a subject suffering from or susceptible to prostate cancer with KARS, EGFR or PTEN mutations with a retinoid X receptor gamma (RXRG) antagonist, wherein the antagonist is compound HX531.

In some embodiments, the invention provides methods of treating a subject suffering from or susceptible to a cancer without KRAS, EGFR or PTEN mutations with a retinoid X receptor gamma (RXRG) agonist, wherein the agonist is of formula I, H, III, IV, V, VI, VII, VIII, or XI. In some embodiments, the invention provides methods of treating a subject suffering from or susceptible to a cancer without KRAS, EGFR or PTEN mutations with a retinoid X receptor gamma (RXRG) agonist, wherein the agonist is of formula VI-n. In some embodiments, the invention provides methods of treating a subject suffering from or susceptible to a cancer without KRAS, EGFR or PTEN mutations with a retinoid X receptor gamma (RXRG) agonist, wherein the agonist is Bexarotene.

In some embodiments, the invention provides methods of treating a subject suffering from or susceptible to a retinoblastoma without KRAS, EGFR or PTEN mutations with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a retinoblastoma without KRAS, EGFR or PTEN mutations with a retinoid X receptor gamma (RXRG) agonist, wherein the agonist is of formula VI-n. In some embodiments, the invention provides methods of treating a subject suffering from or susceptible to a retinoblastoma without KRAS, EGFR or PTEN mutations with a retinoid X receptor gamma (RXRG) agonist, wherein the agonist is Bexarotene.

In some embodiments, the invention provides methods of treating a subject suffering from or susceptible to a cancer without KRAS, EGFR or PTEN mutations and with RB1, BRAF, PIK3CA, PTEN, or EML4-ALK mutation, or combination thereof, with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a cancer without KRAS, EGFR or PTEN mutations and caused by HPV infection, with a retinoid X receptor gamma (RXRG) agonist, wherein the agonist is of formula I, II, III, IV, V, VI, VII, VIII, or XI. In some embodiments, the invention provides methods of treating a subject suffering from, or susceptible to a cancer without KRAS, EGFR or PTEN mutations and with RB1, BRAF, PIK3CA, PTEN, or EML4-ALK mutation, or combination thereof, with a retinoid X receptor gamma (RXRG) agonist, wherein the agonist is of formula VI-n. In some embodiments, the invention provides methods of treating a subject suffering from or susceptible to a cancer without KRAS, EGFR or PTEN mutations and with RB1, BRAF, PIK3CA, PTEN, or EML4-ALK mutation, or combination thereof, with a retinoid X receptor gamma (RXRG) agonist, wherein the agonist is Bexarotene.

In some embodiments, the invention provides methods of treating a subject suffering from or susceptible to a cancer caused by HPV infection and without KRAS, EGFR or PTEN mutations, with a retinoid X receptor gamma (RXRG) agonist, wherein the agonist is of formula I, II, III, IV, V, VI, VII, VIII, or XI. In some embodiments, the cancer is retinoblastoma. In some embodiments, the retinoid X receptor gamma (RXRG) agonist is of formula VI-n. In some embodiments, the retinoid X receptor gamma (RXRG) agonist is Bexarotene.

In some embodiments, the invention provides methods of treating a subject suffering from or susceptible to a retinoblastoma without KRAS, EGFR or PTEN mutations and with RB1, BRAF, P1K3CA, PTEN, or EML4-ALK mutation, or combination thereof, with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a retinoblastoma without KRAS, EGFR or PTEN mutations and with RB1, BRAF, PIK3CA, PTEN, or EML4-ALK mutation, or combination thereof, with a retinoid X receptor gamma (RXRG) agonist, wherein the agonist is of formula VI-n. In some embodiments, the invention provides methods of treating a subject suffering from or susceptible to a retinoblastoma without KRAS, EGFR or PTEN mutations and with RB1, BRAF, PIK3CA, PTEN, or EML4-ALK mutation, or combination thereof, with a retinoid X receptor gamma (RXRG) agonist, wherein the agonist is Bexarotene.

In some embodiments, the invention provides methods of treating a subject suffering from or susceptible to a BRAF mutated melanoma without KRAS or EGFR mutations, with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a BRAF mutated melanoma without KRAS or EGFR mutations, with a retinoid X receptor gamma (RXRG) agonist, wherein the agonist is of formula VI-n. In some embodiments, the invention provides methods of treating a subject suffering from or susceptible to a BRAF mutated melanoma without KRAS or EGFR mutations, with a retinoid X receptor gamma (RXRG) agonist, wherein the agonist is Bexarotene.

In some embodiments, the invention provides methods of treating a subject suffering from or susceptible to a PIK3CA mutated breast cancer without KRAS or EGFR mutations, with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a PIK3CA mutated breast cancer without KRAS or EGFR mutations, with a retinoid X receptor gamma (RXRG) agonist, wherein the agonist is of formula VI-n. In some embodiments, the invention provides methods of treating a subject suffering from or susceptible to a PIK3CA mutated breast cancer without KRAS or EGFR mutations, with a retinoid X receptor gamma (RXRG) agonist, wherein the agonist is Bexarotene.

In some embodiments, the invention provides methods of treating a subject suffering from or susceptible to a HPV infected cervical carcinoma without KRAS or EGFR mutations, with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a HPV infected cervical carcinoma without KRAS or EGFR mutations, with a retinoid X receptor gamma (RXRG) agonist, wherein the agonist is of formula VI-n. In some embodiments, the invention provides methods of treating a subject suffering from or susceptible to a HPV infected cervical carcinoma without KRAS or EGFR mutations, with a retinoid X receptor gamma (RXRG) agonist, wherein the agonist is Bexarotene.

In some embodiments, the invention provides methods of treating a subject suffering from or susceptible to a EML4-ALK fused lung cancer without KRAS or EGFR mutations, with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a EML4-ALK fused lung cancer without KRAS or EGFR mutations, with a retinoid X receptor gamma (RXRG) agonist, wherein the agonist is of formula VI-n. In some embodiments, the invention provides methods of treating a subject suffering from or susceptible to a EML4-ALK fused lung cancer without KRAS or EGFR mutations, with a retinoid X receptor gamma (RXRG) agonist, wherein the agonist is Bexarotene.

In some embodiments, the invention provides methods of treating a subject suffering from or susceptible to a PTEN mutated breast cancer, prostate cancer, SCLC, melanoma, and glioma without KRAS or EGFR mutations, with a retinoid X receptor gamma (RXRG) agonist, wherein the agonist is of formula I, II, III, IV, V, VI, VII, VIII, or XI. In some embodiments, the invention provides methods of treating a subject suffering from or susceptible to a PTEN mutated breast cancer, prostate cancer, SCLC, melanoma, and glioma without KRAS or EGFR mutations, with a retinoid X receptor gamma (RXRG) agonist, wherein the agonist is of formula VI-n. In some embodiments, the invention provides methods of treating a subject suffering from or susceptible to a PTEN mutated breast cancer, prostate cancer, SCLC, melanoma, and glioma without KRAS or EGFR mutations, with a retinoid X receptor gamma (RXRG) agonist, wherein the agonist is Bexarotene.

In some embodiments, the invention provides methods of inhibiting growth of cancer cells with KRAS EGFR, or PTEN mutations, with a retinoid X receptor gamma (RXRG) antagonist, wherein the antagonist is of formula I, II, III, IV, V, VI, VII, VIII, or XI. In some embodiments, the invention provides methods of inhibiting growth of cancer cells with KRAS, EGFR, or PTEN mutations, with a retinoid X receptor gamma (RXRG) antagonist, wherein the antagonist is compound HX531.

In some embodiments, the invention provides methods of inhibiting proliferation of cancer cells with KRAS, EGFR, or PTEN mutations, with a retinoid X receptor gamma (RXRG) antagonist, wherein the antagonist is of formula I, II, III, IV, V, VI, VII, VIII, or XI. In some embodiments, the invention provides methods of inhibiting proliferation of cancer cells with KRAS, EGFR, or PTEN mutations, with a retinoid X receptor gamma (RXRG) antagonist, wherein the antagonist is HX531.

In some embodiments, the invention provides methods of promoting apoptosis of cancer cells with KRAS, EGFR, or PTEN mutations, with a retinoid X receptor gamma (RXRG) antagonist, wherein the antagonist is of formula I, II, III, IV, V, VI, VII, VIII, or XI. In some embodiments, the invention provides methods of promoting apoptosis of cancer cells with KRAS, EGFR, or PTEN mutations, with a retinoid X receptor gamma (RXRG) antagonist, wherein the antagonist is HX531.

In some embodiments, the invention provides methods of suppressing G1/S transition in cancer cells with KRAS, EGFR, or PTEN mutations, with a retinoid X receptor gamma (RXRG) antagonist, wherein the antagonist is of formula I, H, III, IV, V, VI, VII, VIII, or XI. In some embodiments, the invention provides methods of suppressing G1/S transition in cancer cells with KRAS or EGFR mutations, with a retinoid X receptor gamma (RXRG) antagonist, wherein the antagonist is HX531.

In some embodiments, the invention provides methods of inhibiting growth of cancer cells without KRAS or EGFR mutations, with a retinoid X receptor gamma (RXRG) agonist, wherein the agonist is of formula I, II, III, IV, V, VI, VII, VIII, or XI. In some embodiments, the invention provides methods of inhibiting growth of cancer cells without KRAS or EGFR mutations with a retinoid X receptor gamma (RXRG) agonist, wherein the agonist is of formula VI-n. In some embodiments, the invention provides methods of inhibiting growth of cancer cells without KRAS or EGFR mutations with retinoid X receptor gamma (RXRG) agonist, wherein the agonist is compound Bexarotene.

In some embodiments, the invention provides methods of inhibiting proliferation of cancer cells without KRAS or EGFR mutations with a retinoid X receptor gamma (RXRG) agonist, wherein the agonist is of formula I, II, III, IV, V, VI, VII, VIII, or XI. In some embodiments, the invention provides methods of inhibiting proliferation of cancer cells without KRAS or EGFR mutations with a retinoid X receptor gamma (RXRG) agonist, wherein the agonist is of formula VI-n. In some embodiments, the invention provides methods of inhibiting proliferation of cancer cells without KRAS or EGFR mutations with a retinoid X receptor gamma (RXRG) agonist, wherein the agonist is Bexarotene.

In some embodiments, the invention provides methods of promoting apoptosis of cancer cells without KRAS or EGFR mutations with retinoid a X receptor gamma (RXRG) agonist, wherein the agonist is of formula I, II, III, IV, V, VI, VII, VIII, or XI. In some embodiments, the invention provides methods of promoting apoptosis of cancer cells without KRAS or EGFR mutations with a retinoid X receptor gamma (RXRG) agonist, wherein the agonist is of formula VI-n. In some embodiments, the invention provides methods of promoting apoptosis of cancer cells without KRAS or EGFR mutations with a retinoid X receptor gamma (RXRG) agonist, wherein the agonist is Bexarotene.

In some embodiments, the invention provides methods of delaying S phase progression and G2/M transition in cancer cells without KRAS or EGFR mutations with a retinoid X receptor gamma (RXRG) agonist, wherein the agonist is of formula I, II, III, IV, V, VI, VII, VIII, or XI. In some embodiments, the invention provides methods of delaying S phase progression and G2/M transition in cancer cells without KRAS or EGFR mutations with a retinoid X receptor gamma (RXRG) agonist, wherein the agonist is of formula VI-n. In some embodiments, the invention provides methods of delaying S phase progression and G2/M transition in cancer cells without KRAS or EGFR mutations with a retinoid X receptor gamma (RXRG) agonist, wherein the agonist is of formula Bexarotene.

In some embodiments, the invention provides methods of modulating functions of Treprec-Xu complex in cancer, using one or more compounds of formula I, II, III, IV, V, VI, VII, VIII, or XI. In some embodiments, the invention provides methods of modulating functions of Treprec-Xu complex in cancer by inhibiting or promoting association or dissociation of one or more components of the complex with each other and/or with the complex, using one or more compounds of formula I, II, III, IV, V, VI, VII, VIII, or XI.

In one aspect, the present invention provides methods to modulate RXRG function in cancer, using one or more compounds of formula I, II, III, IV, V, VI, VII, VIII, or XI.

Methods of the present invention may be used in vitro or in vivo. The methods may be particularly useful in the treatment of cancers as described herein in vivo. However, inventive methods described above may also be used in vitro for research or clinical purposes (e.g., determining the susceptibility of a patient's disease to a compound, researching the mechanism of action, elucidating a cellular pathway or process).

In certain embodiments, methods of the present invention include combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with compounds described herein include surgery, radiotherapy (γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. Additionally, the present invention also encompasses the use of certain cytotoxic or anticancer agents currently in clinical trials and which may ultimately be approved by the FDA (including, but not limited to, epothilones and analogues thereof and geldanamycins and analogues thereof).

In certain embodiments, methods are useful in treating a subject in clinical remission. In some embodiments, the subject has been treated by surgery and may have limited unresected disease.

### EXEMPLIFICATION

### General Procedures and Examples

Cell Culture

Colon, lung, pancreatic cancer, retinoblastoma and other cell lines were cultured in culture medium consisting of IMDM (Medium Lab, MSKCC, NY) with 10% FBS (SH3008803, HyClone, Thermo Scientific), 2 mM glutamine (Invitrogen), 55 μM beta-mercaptoethanol (Invitrogen), 1% Penicillin/streptomycin (Invitrogen), and 2.5 μg/ml Plasmocin (Invivogen), at 37° C. in a humidified incubator with 5% CO2. Melanoma cell lines were culture in RPMI with above additives.

MDMX and MDM2 Promoter Luciferase Assay:

RB177 cells were plated at 2.5×10⁵ cells per well of a 24-well dish, in IMDM plus 10% FBS, and transfected with 2 μl of Lipofectamine 2000 (Invitrogen), 0.04 μg of pRL-TK (Promega), and 0.8 μg of either pGL3 (Promega) or pGL3-HDM2-P2-luc-02 (Phelps, M., Darley, M., Primrose, J. N., and Blaydes, J. P. (2003). p53-independent activation of the hdm2-P2 promoter through multiple transcription factor response elements results in elevated hdm2 expression in estrogen receptor alpha-positive breast cancer cells. Cancer Res 63, 2616-2623), or pGL2 (Promega) or pGL2-HDMX-luc (Gilkes D M, Pan Y, Coppola D, Yeatman T, Reuther G W, Chen J. Regulation of MDMX expression by mitogenic signaling. Mol Cell Biol. 2008; 28:1999-2010). The compounds to be determined were dissolved in DMSO, and diluted in medium, then added to transfected cells with DMSO as the control at 24 hours after transfection. The cells were harvested at 72 hours, collected by centrifugation, washed with 1 ml PBS, resuspended and lysed in 100 μl PLB (Promega). The Renilla and firefly luciferase activities were measured using the Stop-and-Glow system and ProMega luminometer. Firefly luciferase activity was calculated and normalized to Renilla luciferase activity. If the compound promoted pGL2-HDMX-luc or pGL3-HDM2-P2-luc-02 firefly luciferase activity, then it is an RXRG agonist; if the RXRG ligand reduces firefly luciferase activity, then it is an RXRG antagonist.

Direct Co-Immunoprecipitation (Co-IP) and Western Blot (WB) Analysis

The Thermo Scientific Pierce Antibody Clean-up Kit (44600) was used for removing BSA and gelatin (up to 1%) from IgG samples. Pierce® Direct IP Kit (26148) was used for antigen immunoprecipitation by directly immobilizing purified antibodies onto an amine-reactive agarose support according to its manual with some modification. Immobilizing the antibody can reduce antibody contamination in purified antigens. After 20 μg of each antibody was coupled onto the AminoLink Resin, the cell lysate from retinoblastoma Y79 and RB177 or colon cancer HCT116 were incubated with the immobilized antibody to form the immune complex. The complex was washed to remove non-bound material, and a low pH elution buffer was used to dissociate the bound antigen from the antibody. The concentration of NaCl in IP Lysis/Wash Buffer was increased to 0.25 M to enhance the IP of Rb protein. Western blot analysis was similar to described protocol. In order to further reduce the background caused by contaminated antibody from IP, different species of antibodies were used for detection of antigens after Co-IP. If we could not find different antibodies with different species to reduce the background of heavy chain (about 55 kD), we used the HRP conjugated protein A (1:50.00, Thermo Scientific) to reduce the background. Of note, HRP conjugated protein A could only bind to hybridized double heavy chains, but not degenerated single heavy chain.

Immunofluorescence (IF) Analysis

Colon cancer or lung cancer cells were passaged to dishes with coverslips, and incubated in a humidified incubator at 5% CO2 and 37° C. for 1-2 days. The medium was removed and the cells fixed in 4% PFA/PBS for 5 min, gently rinsed with 0.15 M NaCl/20 mM Tris (pH 8.0; TBS), dried, and stored at −20° C. On 5-7 days after RB1 or RXRG-KD, HCT116 cells were trypsinized and spread on poly-L-lysine coated slides with medium, and incubated for 3-4 hours before fixation. After treatment of RXRG ligands or RXRG knockdown, retinoblastoma cells were spread on poly-L-lysine coated slides with medium, and incubated for 3-4 hours before fixation.

Staining with mouse antibodies: Cells were treated with 1 mM EDTA/PBS for 5 min at room temperature and, washed with PBS. Cells were treated with ABC kit reagent A (Vector Laboratories, Burlingame, Calif.) in PBS for 15 min, washed in TBS, treated with ABC kit reagent B (Vector Laboratories) in TBS for 15 min, washed in PBS, blocked and permeabilized for 20 min in block 1 (2.5% horse serum, 2.5% donkey serum, 2.5% human serum, 1% BSA, 0.1% Triton-X-100, and 0.05% Tween-20 in TBS; filtered with 0.22 um filter), incubated in the above mouse primary antibodies in block 1 overnight at 4° C., and washed in PBS. They were then incubated in biotinylated horse anti-mouse antibody (BA-2000, Vector Laboratories; 1:135) in block 1 for 30 min, washed in PBS, incubated with FITC-conjugated streptavidin (Vector Laboratories; 1:175) in PBS, and washed with PBS.

Co-staining analyses with other antibodies: On completing the first staining reaction as described above, cells were incubated in block 1 for 20 min, incubated overnight with primary antibody in block 1, washed in PBS, incubated for 30 min in block 1 with Cy3 or Cy5 conjugated secondary antibody, and washed in PBS. Sections were then stained with 4′,6′-diamino-2-phenylindole (DAPI) in PBS, mounted in VECTASHIELD Mounting Media (Vector Labs), and analyzed by inverted immunofluorescent microscopy (Axioplan2 Imaging, Carl Zeiss MicroImaging, LLC). The specificity of all antibodies used in co-staining analyses was confirmed by staining in parallel with control mouse or rabbit IgG. The specificity of other antibodies was confirmed by staining in parallel with a same species control or with no primary antibody.

Some of the antibodies used are listed below.

TABLE 1a
Mouse antibodies used for Co-IP, IF, and Western blot (WB).

| Mouse | | | | | |
|---|---|---|---|---|---|
| Antibodies | Catalog No. | Clone | Titer | Company | Application |
| Alpha-tubulin | t9026 | DMIA | 1:10,000 | Sigma | WB |
| | | | WB | | |
| CDC25C | MS-751-P0 | 25C14 | (1:100) | NeoMarkers | IF, WB |
| CDH1 | MS-1116-p0 | DH01 | (1:100) | NeoMarkers | IF, WB |
| CDK1 | 610038 | | (1:100) | BD- | IF, WB |
| | | | | Transduction | |
| | | | | labs | |
| CDK2 | sc-6248 | D-12 | (1:100) | Santa Cruz | IF, WB |
| Cyclin B1 | MS-338-P0 | V152 | (1:100) | NeoMarkers | IF, WB, IP |
| Cyclin B1 | SC-752 | H-433 | (1:100) | Santa Cruz | IF, WB |
| Cyclin D1 | sc-8396 | A-12 | (1:200) | Santa Cruz | IF, WB |
| E2F1 | SC-251 | KH95 | (1:100) | Santa Cruz | IF, WB |
| Fibrillarin | MA3-16771 | | (1:100) | Pierce | IF, WB |
| MDM2 | SC-965 | SMP-14 | (1:100) | Santa Cruz | IF, WB, IP |
| p130 | MS-866-P0 | Ab- | (1:100) | NeoMarkers | IF, WB |
| | | 2(130P215) | | | |
| p21 | OP79 | | (1:100) | Calbiochem | IF, WB |
| p27 | 610241 | | (1:100) | BD- | IF, WB |
| | | | | Transduction | |
| | | | | labs | |
| p53 | SC-126 | DO-1 | (1:100) | Santa Cruz | IF, WB, IP |
| Phospho-Rb- | 558389 | | (1:500) | BD- | IF, WB |
| s807/s811 | | | | PharMingen | |
| PP1-C | SC-7482 | E-9 | (1:100) | Santa Cruz | IF, WB, IP |
| PP2A-C-a | 610556 | 46 | (1:100) | BD | IF, WB |
| | | | | Biosciences | |
| PTTG1(Pds1) | MS-1511-P1 | DCS-280 | (1:100) | NeoMarkers | IF, WB |
| RXRG | MS-1343-P0 | Ab-3(1373) | (1:100) | NeoMarkers | IF, WB |
| Rb | 554136 | G3-245 | (1:100) | BD- | IF, WB |
| | | | | PharMingen | |
| Rb | D36802 | Rb-Ab5 | (1:500) | Calbiochem | IF, WB |
| TRB1 | MA1-216 | J-52 | (1:100) | Pierce | IF, WB, IP |
| Mouse | | | | | |
| Antibodies | Catalog No. | Clone | Titer | Company | Application |
| Alpha-tubulin | t9026 | DMIA | 1:10,000 | Sigma | WB |
| | | | WB | | |
| CDC25C | MS-751-P0 | 25C14 | (1:100) | NeoMarkers | IF, WB |
| CDH1 | MS-1116-P0 | DH01 | (1:100) | NeoMarkers | IF, WB |
| CDK1 | 610038 | | (1:100) | BD- | IF, WB |
| | | | | Transduction | |
| | | | | labs | |
| CDK2 | sc-6248 | D-12 | (1:100) | Santa Cruz | IF, WB |
| Cyclin B1 | MS-338-P0 | V152 | (1:100) | NeoMarkers | IF, WB, IP |
| Cyclin B1 | SC-752 | H-433 | (1:100) | Santa Cruz | IF, WB |
| Cyclin D1 | sc-8396 | A-12 | (1:200) | Santa Cruz | IF, WB |
| E2F1 | SC-251 | KH95 | (1:100) | Santa Cruz | IF, WB |
| Fibrillarin | MA3-16771 | | (1:100) | Pierce | IF, WB |
| p130 | MS-866-P0 | Ab- | (1:100) | NeoMarkers | IF, WB |
| | | 2(130P215) | | | |
| p21 | OP79 | | (1:100) | Calbiochem | IF, WB |
| p27 | 610241 | | (1:100) | BD- | IF, WB |
| | | | | Transduction | |
| | | | | labs | |
| Phospho-Rb- | 558389 | | (1:500) | BD- | IF, WB |
| s807/s811 | | | | PharMingen | |
| PP1-C | SC-7482 | E-9 | (1:100) | Santa Cruz | IF, WB, IP |
| PP2A-C-a | 610556 | 46 | (1:100) | BD | IF, WB |
| | | | | Biosciences | |
| PTTG1(Pds1) | MS-1511-P1 | DCS-280 | (1:100) | NeoMarkers | IF, WB |
| Rb | 554136 | G3-245 | (1:100) | BD- | IF, WB |
| | | | | PharMingen | |
| Rb | D36802 | Rb-Ab5 | (1:500) | Calbiochem | IF, WB |
| TRB1 | MA1-216 | J-52 | (1:100) | Pierce | IF, WB, IP |

TABLE 1b
Goat or Chicken antibodies used for Co-IP, IF, and Western blot (WB).

| | Catalog | | | | |
|---|---|---|---|---|---|
| Antibodies | No. | Clone | Titer | Company | Application |
| CDC14B- | GW21523 | chicken | (1:100) | SIGMA | IF, WB, IP |
| Ab1 | | | | | |
| p-Rb (Ser | sc-16671 | goat | (1:50) | Santa Cruz | IF, WB |
| 249/Thr 252) | | | | | |

TABLE 2A
Rabbit Antibodies used for Co-IP, IF, and Western blot (WB).

| Rabbit Antibodies | Catalog No. | Clone | Titer | Company | Application |
|---|---|---|---|---|---|
| Biotin-phosph-histone 3 | 16-189 | | (1:200) | Upstate, | IF |
| | | | | Millipore | |
| CDC14B-Ab2 | ab104415 | | (1:100) | Abcam | IF, IP |
| cdh1 fzr | 34-2000 | | (1:100) | Invitrogen | IF, WB, IP |
| CDK1 (P34) | SC-954 | | (1:100) | Santa Cruz, CA | IF |
| Cyclin A | sc-751 | H432 | (1:100) | Santa Cruz, CA | IF, WB |
| Cyclin E | SC-481 | M20 | (1:100) | Santa Cruz, CA | IF, WB, IP |
| Cyclin E | SC-198 | C-19 | (1:100) | Santa Cruz, CA | IF, WB, IP |
| Emil (FBXO5) | HPA029048 | | (1:100) | Sigma, Atlas | IF, WB, IP |
| p107 | sc-318 | c18 | (1:100) | Santa Cruz, CA | IF, WB, IP |
| p130 | sc-317 | C-20 | (1:100) | Santa Cruz, CA | IF, WB, IP |
| Phospho-CDC25C | #4901S | 63F9 | (1:200) | Cell Signaling | IF, WB |
| | | | | Technology | |
| Phospho-p107-S975 | SC-130209 | | (1:100) | Santa Cruz, CA | IF, WB, IP |
| Phospho-p130-S952 | 2272-1 | | (1:300) | Epitomics | IF, WB |
| Phospho-PP1-alpha- | #2581S | T320 | (1:300) | Cell Signaling | IF, WB |
| catalytic | | | | Technology | |

TABLE 2B
Rabbit Antibodies used for Co-IP, IF, and Western blot (WB).

| Rabbit Antibodies | Catalog No. | Clone | Titer | Company | Application |
|---|---|---|---|---|---|
| Phosph-Rb(s795) | 9301S | | (1:100) | Cell Signaling | IF, WB, IP |
| | | | | Technology | |
| Phosph- | 9306s | | (1:100) | Cell Signaling | IF, WB, IP |
| Rb(Thr373) | | | | Technology | |
| PP2A-C | SC-14020 | FL-309 | (1:100) | Santa Cruz, | IF, WB, IP |
| | | | | CA | |
| PP2A-C | #2259 | 52F8 | (1:500)WB | Cell Signaling | WB |
| | | | | Technology | |
| p-PP2A-C-Tyr307 | P67775 | E155 | (1:100) | Epitomics, Inc. | IF, WB |
| RXRG | E4331 | | (1:100) | Spring | IF, WB, IP |
| | | | | Bioscience, | |
| SKP2 | SC-7164 | H-435 | (1:100) | Santa Cruz, | IF, WB, IP |
| | | | | CA | |
| SKP2 | 51-1900 | | (1:100) | Zymed | IF, WB |
| TRB1 | PA1-213A | | (1:100) | Pierce | IF, WB |
| TRIP11/230 | HPA002570- | | (1:100) | Sigma Atlas | IF, WB |
| | 100UG | | | | |
| TRIP11/230 | A301-187A | | (1:100) | Bethyl | IF, WB |
| TRIP11/230 | A301-188A | | (1:100) | Bethyl | IF, WB |
| TRβ2 | Ng et al., | | (1:100) | Forrest D | IF, WB |
| | 2009 | | | | |
| TRβ2-Ab1 | SC-67123 | | (1:2,000)WB | Santa Cruz, | WB, IP |
| | | | | CA | |
| TRβ2-Ab2 | 06-540 | | (1:2,000)WB | Upstate, | WB |
| | | | | Millipore | |

TABLE 3
Secondary Antibodies used for Co-IP, IF, and Western blot (WB)

| | | | | | | |
|---|---|---|---|---|---|---|
| Secondary | | | | | | Applica- |
| antibodies | Species | Catalog No. | Clone | Titer | Company | tion |

| | | | | | |
|---|---|---|---|---|---|
| Biotin- horse | Horse | BA-2000 | (1:135) | Vector | IF |
| anti-mouse | | | | Laboratories | |
| Biotin- goat | goat | BA-1000 | (1:135) | Vector | IF |
| anti-rabbit | | | | Laboratories | |
| FITC- | No | SA-5001 | (1:175) | Vector | IF |
| streptavidin | | | | Laboratories | |
| Cy3 donkey | donkey | 715-165-150 | (1:150) | Jackson | IF |
| anti-mouse | | | | Laboratories | |
| Cy3 donkey | donkey | 711-165-152 | (1:150) | Jackson | IF |
| anti-rabbit | | | | Laboratories | |
| Cy3 donkey | donkey | 711-167-003 | (1:151) | Jackson | IF |
| anti-rabbit Fab | | | | Laboratories | |
| Cy5- Donkey | donkey | 705-175-147 | (1:150) | Jackson | IF |
| anti-goat | | | | | |
| Cy5 donkey | donkey | 715-175-151 | (1:150) | Jackson | IF |
| anti-mouse | | | | Laboratories | |
| Cy5 donkey | donkey | 711-175-152 | (1:150) | Jaekson | IF |
| anti-rabbit | | | | Laboratories | |
| DyLight 649 | donkey | 705-495-147 | (1:150) | Jackson | IF |
| donkey anti- | | | | Laboratories | |
| goat | | | | | |
| HRP sheep | sheep | NXA931 | 1:25,000 | Amersham | WB |
| anti-mouse | | | | Biosciences | |
| HRP sheep | sheep | NA934 | 1:150,000 | Amersham | WB |
| anti-rabbit | | | | Biosciences | |
| HRP donkey | donkey | 705-035-003 | 1:50,000 | Jackson | WB |
| anti-goat | | | | Laboratories | |
| HRP-Protein A | No | NA93432400 | 1:50,000 | Thermo | WB for IP |
| | | | | Scientific | |

shRNA Knock-Down

Effective pLKO lentiviral shRNA vectors were from the TRC library (Open Biosystems or MSKCC High-Throughput Drug Screening Facility) and in the text they are designated by sh followed by the name of the target protein and the last 3-4 digits of the Oligo ID or SKI ID number. The pLKO scrambled control was Addgene plasmid 1864 (Sarbassov et al., 2005). Concentrated pLKO lentiviral shRNA vectors were produced in 293T cells using pLKO-shRNA (20 ug), helper constructs pVSVg (10 ug) and delta-8.9 (20 ug), and 90 ul Lipofectamine 2000 (Invitrogen) in 15 cm dishes. Virus harvested 48 and 72 h after transfection was combined, concentrated ˜50-fold by centrifugation and resuspension in growth medium, and ˜500 μl of concentrated virus used to infect 2-5×10⁵ retinoblastoma, neuroblastoma, and colon cancer HCT116 cells suspended in 24 or 12 well plates with 500 μl of growth medium, in the presence of 4 μg/ml polybrene. Infected cells were diluted 3-fold in growth media after 24 h, and cells were selected with 1.4-3 μg/ml puromycin for 48-72 h, starting 48 h after infection, and subsequently fed every 3 days by replacing two-thirds of the medium. Cell number was counted every 5-7 days after RB1 or RXRG knockdown to determine the cell growth curves.

TABLE 4
RB1 shRNA constructs.

| shRNA | Gene | | | | | |
|---|---|---|---|---|---|---|
| Name | Name | Protein | Oligo ID | SKI ID | Accession | Catalog No. |
| 733* | RB1 | Rb | TRCN000 | SK1-RSI- | NM_000321 | RHS3979-9607552 |
| | | | 0040163 | 192733 | | |
| 737* | RB1 | Rb | TRCN000 | SKI-RSI- | NM_000321 | RHS3979-9607556 |
| | | | 0040167 | 192737 | | |

TABLE 5
RXRG shRNA constructs.

| | | | | Homol- | Vector | | |
|---|---|---|---|---|---|---|---|
| shRXRG | Oligo ID | Accession | Type | ogy | type | Vector | Catalog No. |
| 639 | TRCN000002 | NM_006917 | shRNA | Hs | Lentiviral | pLKO.l | RHS3979-9589047 |
| | 1639 | | | | | | |
| 640 | TRCN000002 | NM_006917 | shRNA | Hs | Lentiviral | pLKO.l | RHS3979-9589048 |
| | 1640 | | | | | | |
| 641 | TRCN000002 | NM_006917 | shRNA | Hs | Lentiviral | pLKO.l | RHS3979-9589049 |
| | 1641 | | | | | | |

Cell Cycle Synchronization and Cell Cycle Analysis

Cell Preparation:

After lentivirus infection or drug treatment, cells were collected for cell cycle analysis, cell counting, RNA isolation, quantitative PCR, and western blot. For cell cycle analysis, retinoblastoma, neuroblastoma, lung, pancreatic, and colon cancer cells were collected and dissociated on days 5, 7, 9, 11, and 16 after infection. Cells were fixed by 70% cold ethanol alcohol while vortex, and put in −20° C. for storage.

Propidium Iodide Staining and Flow Cytometry Analysis:

The tubes with cells were taken out of the freezer and alcohol was removed by centrifugation. 150 ul of 0.06 mg/ml propidium iodide with 0.1% NF40 was added to the cells, and then 100 ul of 2 mg/ml of RNase was added to the cells. These were mixed well and incubated at 37° C. for 30 min. The cell ploidy according to DNA contents were measured in FACSCalibur with FL3-Width and FL3-Height as the parameter. Flowjo was used for analysis of the cell cycle changes.

Cell Cycle Synchronization and Cell Cycle Analysis with Drug Treatment

Cell Preparation:

After drug treatment, cells were collected for cell cycle analysis, RNA isolation, quantitative PCR, western blot, and immunofluorescence. For cell cycle analysis, retinoblastoma, neuroblastoma, lung, pancreatic, and colon cancer cells were collected and dissociated on days 3, 5, 7, 9, 11, and 16 after treatment. Cells were fixed by 70% cold ethanol alcohol while vortex, and put in −20° C. for storage. For short time course of cell cycle analysis, 5-10 uM HX531, Bexarotene, 2 uM 9cis RA, or DMSO were added to retinoblastoma or A549 cells. At 24 hours after treatment, 4 ug/ml Aphidicolin was added to cells with compounds for 24 hr. Aphidicolin was removed by centrifugation and PBS wash, and then added to new wells with fresh medium and compounds. Cells were collected and fixed before addition of aphidicolin, immediately after removal of aphidicolin, and every two hours thereafter until 16-18 hours.

Propidium Iodide Staining and Flow Cytometry Analysis:

The tubes with cells were taken out of the freezer and alcohol was removed by centrifugation. 150 ul of 0.06 mg/ml propidium iodide with 0.1% NF40 was added to the cells, and then 100 ul of 2 mg/ml of RNase was added to the cells. These were mixed well and incubated at 37° C. for 30 min. The cell ploidy according to DNA contents were measured in FACSCalibur with FL3-Width and FL3-Height as the parameter. Flowjo was used for analysis of the cell cycle changes.

Western Blot Analysis:

Immunoblotting of LKO-shRXRG infected (D5) and puromycin selected retinoblastoma, colon cancer, and neuroblastoma cells were gently pelleted, suspended in PBS, re-pelleted, and lysed in ELB (150 mM NaCl, 50 mM HEPES pH 7.4, 0.1% NF40, 5 mM EDTA, 2 mM DTT, 1 mM phenylmethylsulfonyl fluoride, 10 mM NaF, 1 mM NaVO4, Thermo Scientific Halt phosphatase inhibitor cocktail and protease inhibitor cocktails). Sample buffer was added and heated at 95° C. for 5 min before PAGE or for storage at −80° C. Frozen samples were heated again for 5 min before PAGE. Proteins were separated on 4-20% Ready Gel Tris-HCl (Jule Biotechnologies INC), transferred to Hybond-ECL nitrocellulose membrane (Amersham Biosciences), and membranes incubated with diluted target antibodies (mostly 1:2000) and α-tubulin (DM1A, K4805, Sigma; 1:10,000), or γ-tubulin (GTU-88, T6557, Sigma; 1:2000), and detected with HRP conjugated sheep anti-mouse (NXA931, 1:25,000) or anti-rabbit (NA934, 1:150,000, Amersham Biosciences), using ECL Advance Western Blotting Detection Kit (Amersham Biosciences) and HyBlot CL X-Ray film (Denville Scientific Inc).

Drug Treatment on Cancer Cell Lines:

Colon, lung, pancreatic, breast, prostate cancer cell lines and other cancer cell lines were cultured in 10 cm dishes or 24 well plates with complete IMDM or RPMI (for melanoma) with 30-50% confluence. Compounds such as HX531, UVI3003, PA452, PA024, Bexarotene, 9cis RA, and all-trans RA were dissolved in DMSO, diluted in medium, added to medium with different concentrations and mixed immediately. DMSO was used for control. After 3-6 days treatment, cells were collected and counted with trypan blue. Dose-effectiveness curves were generated with series dilution of compounds in medium for treatment of cells.

TABLE 6
Cell line information.

| Cancer type | Name | Mutations |
|---|---|---|
| NSCLC | H1650 | EGFR, CDKN2A, TP53 |
| NSCLC | H1975 | EGFR, CDKN2A, TP53, PIK3CA |
| NSCLC | H3255 | EGFR |
| NSCLC | H820 | EGFR |
| NSCLC | A549 | KRAS, CDKN2A, STK11, |
| | | SMARCA |
| NSCLC | H2030 | KRAS, TP53, STK11, SMARCA4 |
| NSCLC | H358 | KRAS |
| Pancreatic | PC0201 | KRAS |
| Pancreatic | PC1102 | KRAS |
| Pancreatic | PC1019 | KRAS |
| Colon Cancer | CCCL13 | KRAS |
| Colon Cancer | CCCL18 | KRAS |
| Colon Cancer | HCT116 | KRAS, CDKN2A, PIK3CA, |
| | | CTNNB1, MLH1 |
| NSCLC | NCI-H460 | KRAS, CDKN2A, PIK3CA, STK11 |
| NSCLC | H1755 | BRAF, CDKN2A, STK11, TP53 |
| Melanoma | OCM1 | BRAF |
| Breast Cancer | MDA-MB-468 | RB1, PTEN, SMAD4, TP53 |
| Breast Cancer | MDA-MB-453 | CDH1, PIK3CA, TP53, HER2+ |
| SCLC | NCI-H209 | RB1, TP53, THRB |
| SCLC | NCI-H446 | RB1, TP53, THRB, PTEN |
| Melanoma | C918 | ? |
| Hepatoma | HepG2 | ? |
| Colon Cancer | CCCL6 | TP53 |
| NSCLC | H2228 | EML4-ALK, RB1, TP53 |
| NSCLC | H3122 | EML4-ALK |
| NSCLC | NCI-H1299 | NRAS |
| Neuroblastoma | SKN-BE(2) | MycN, TP53, NF1 |
| Neuroblastoma | IMR32 | MycN |
| Melanoma | M21 | BRAF? |
| Prostate Cancer | LNCaP | MSH2, PTEN |
| Prostate Cancer | DU145 | CDKN2A, MLH1, RB1, STK11, |
| | | TP53 |
| Prostate Cancer | PC3 | PTEN, TP53 |
| Breast Cancer | MCF7 | CDKN2A, PIK3CA |
| Cervical Cancer | Hela | HPV infection |
| Osteosarcoma | U2OS | RB1 wild type |
| Osteosarcoma | Saos2 | RB1, TP53 |
| Retinoblastoma | RB176 | RB1 |
| Retinoblastoma | WERI | RB1 |
| Retinoblastoma | RB176 | RB1 |
| Retinoblastoma | Y79 | RB1, MYCN |
| Retinoblastoma | RB177 | RB1 |
| Fetal Fibroblast | WI38 | No |

### 1. The Treprec-Xu Complex

### S-Phase Promoting Complex, SPC

### Example 1

### Identification of SPC and Related Protein-Protein Interaction

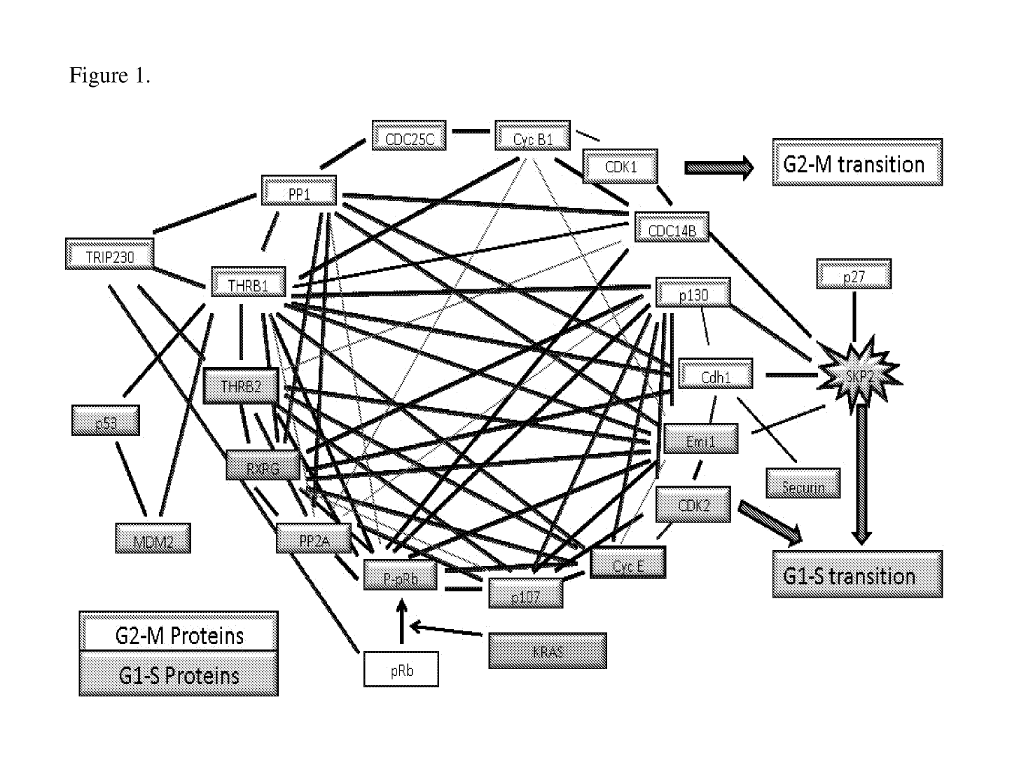

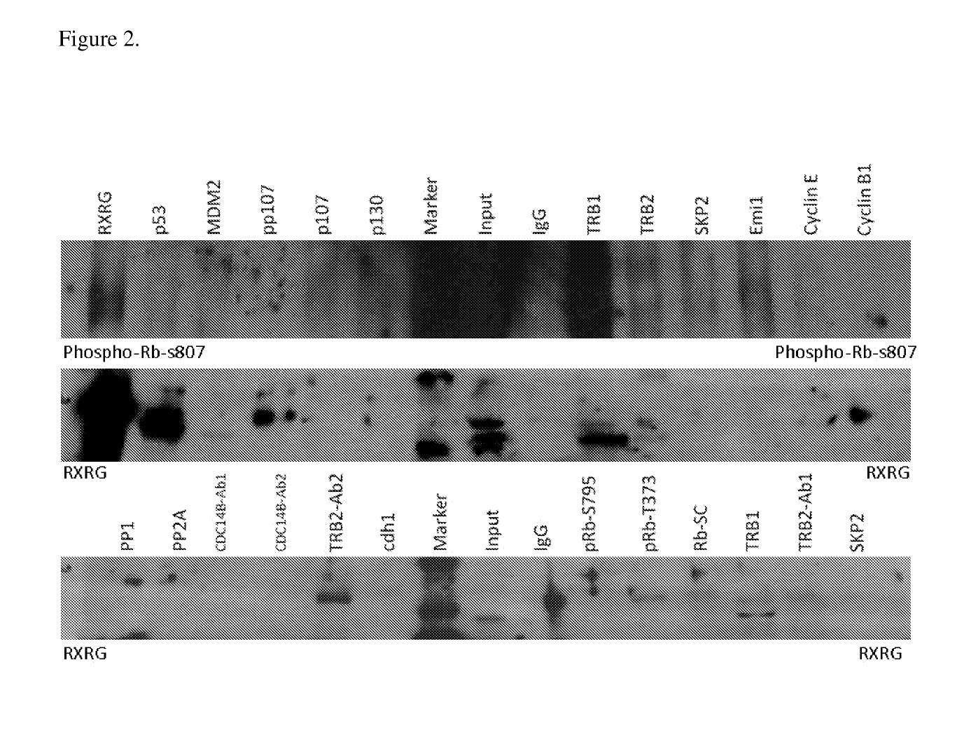

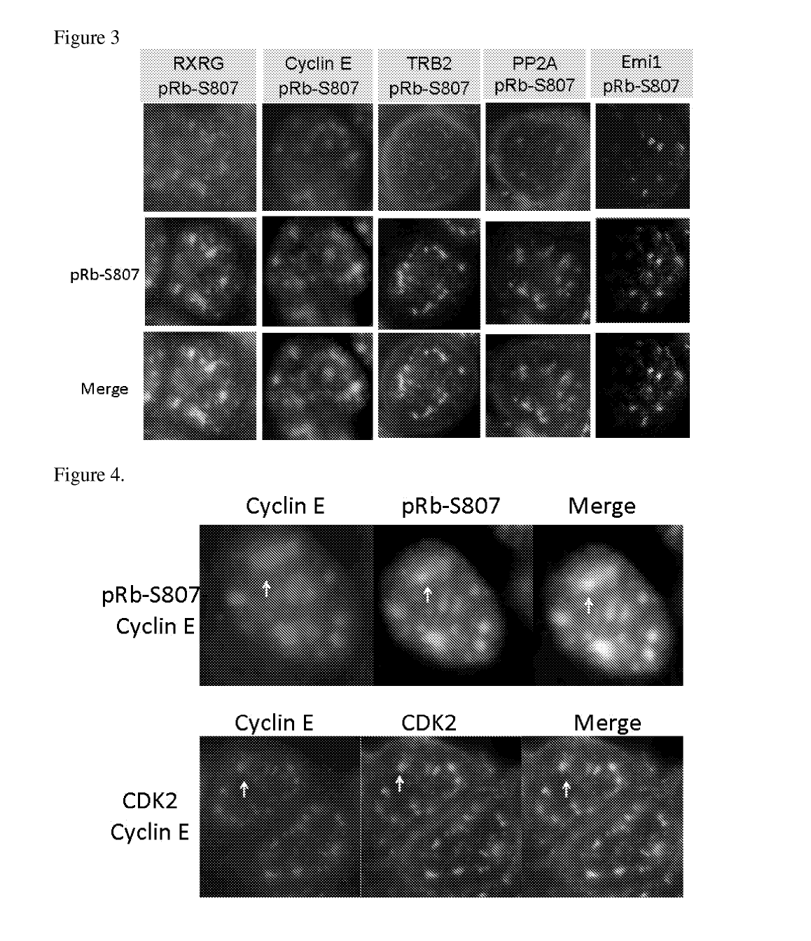

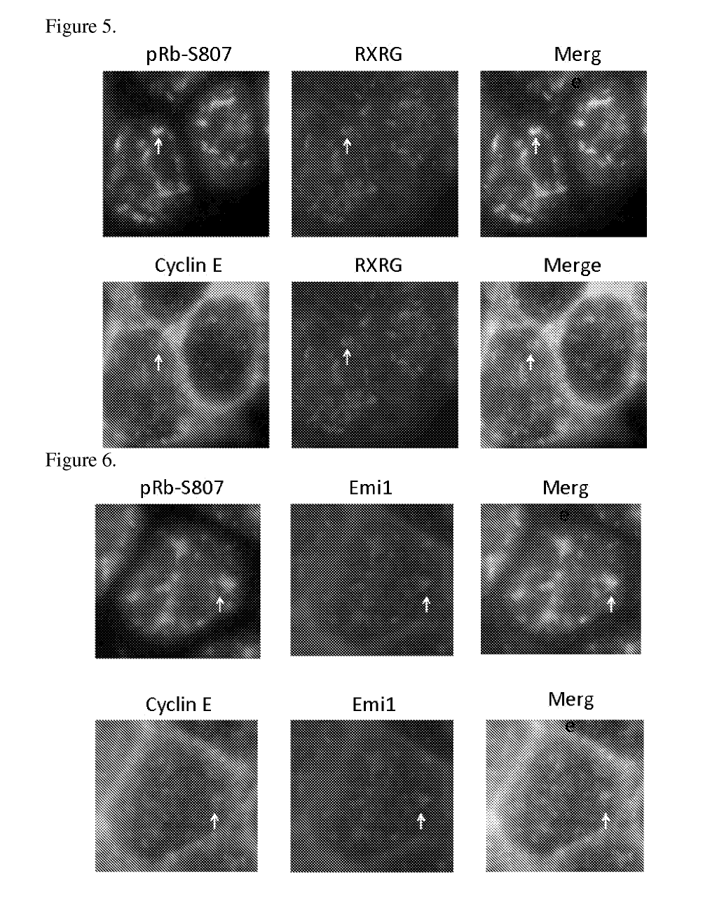

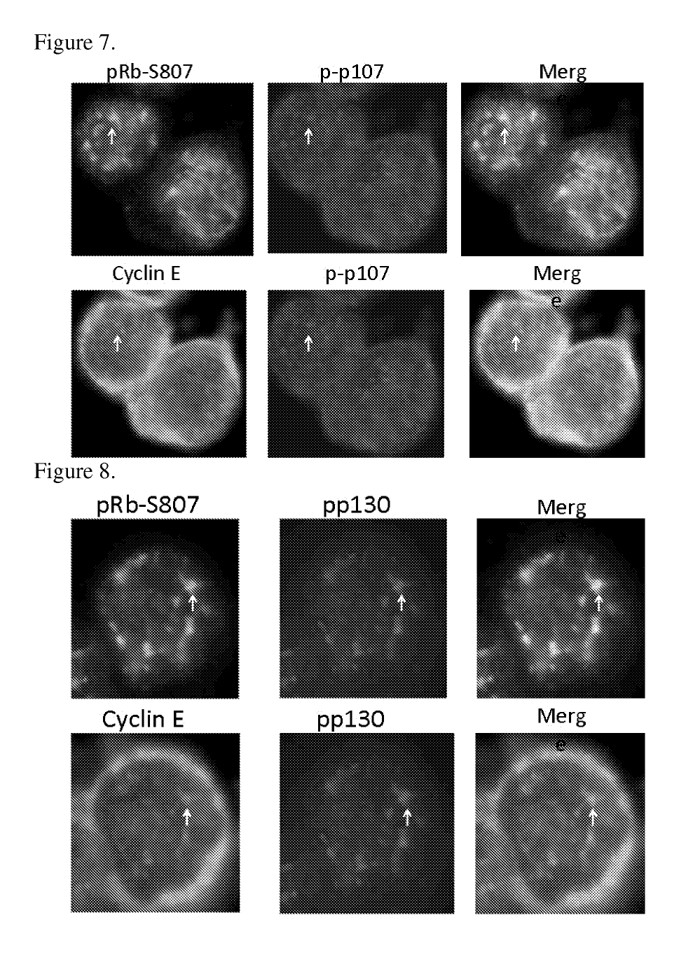

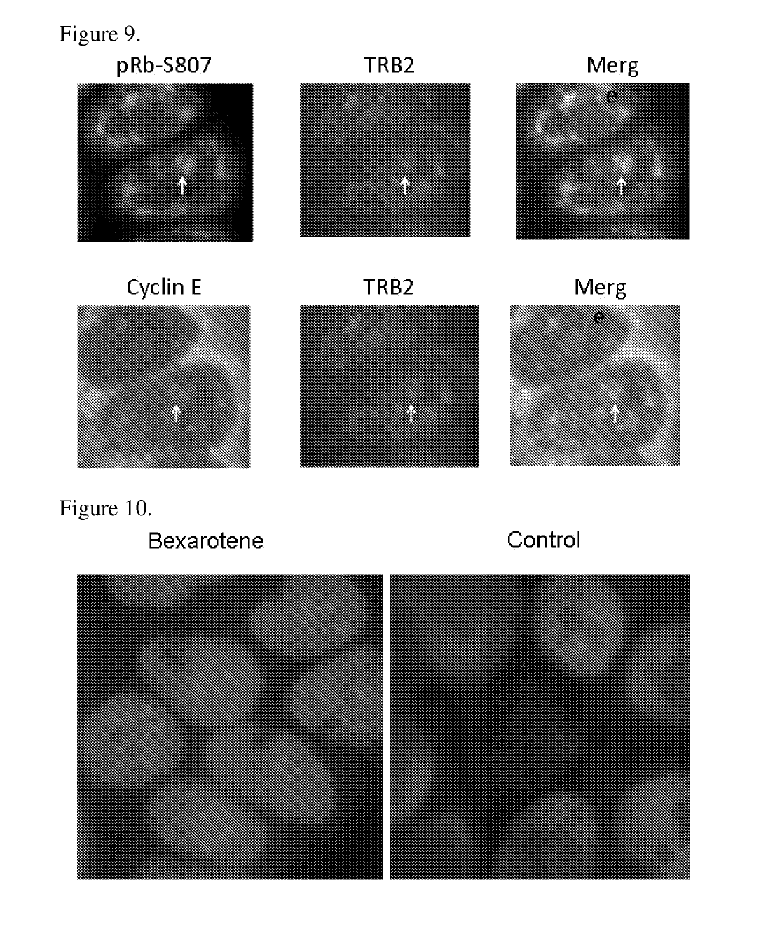

Using Co-Immunoprecipitation and Immunofluorescence, we identified the Treprec-Xu (SPC) complex and extensively investigated protein-protein interactions between its members. Among others, RXRG interacted with phosphorylated RB, phospho-p107, Cyclin E, PP1, P53, TRB2, and TRB1. The results are illustrated in FIGS. 1-9.

### Example 2

### Knock-Down of RXRG LED to Dissociation of SPC and Cell Cycle Arrest

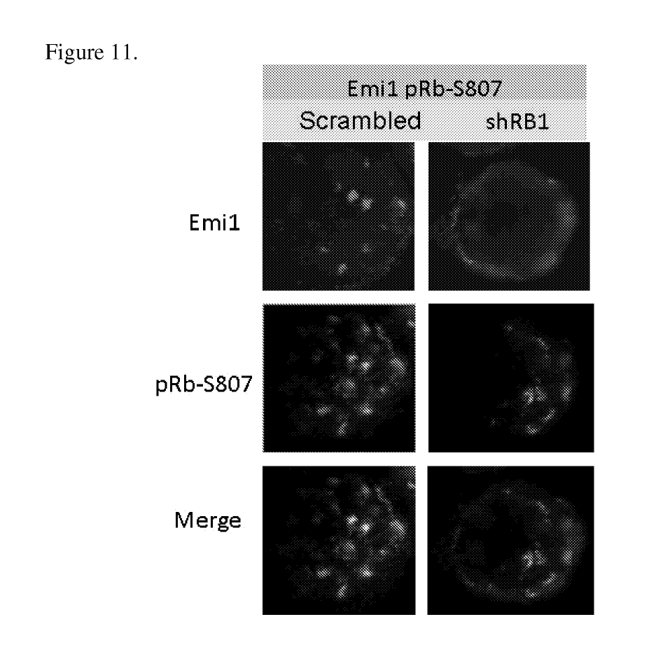

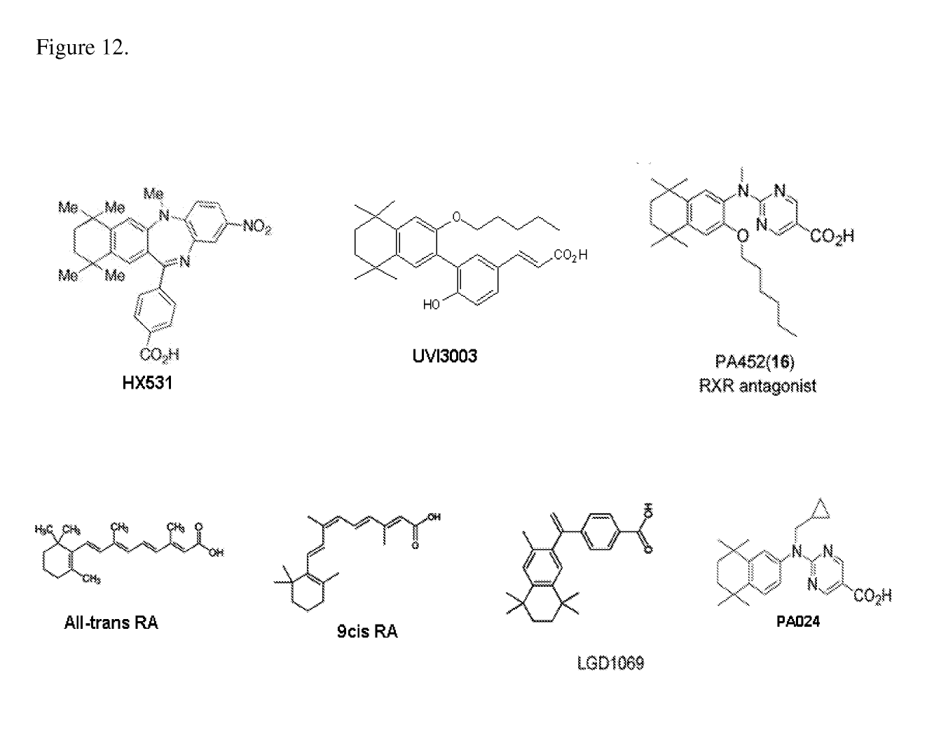

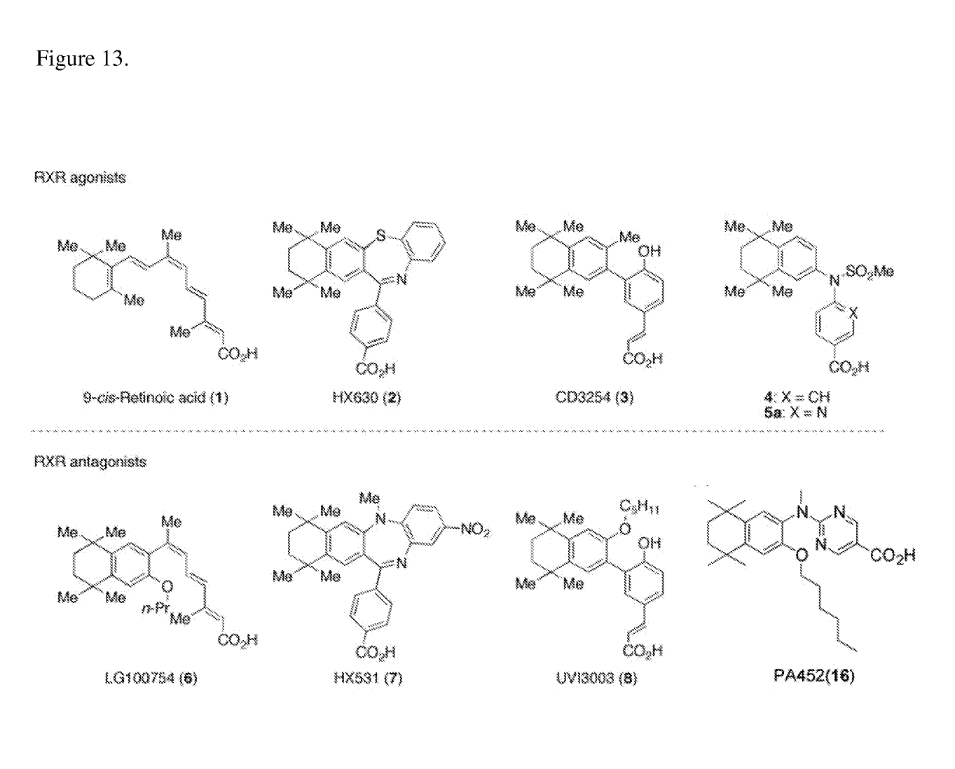

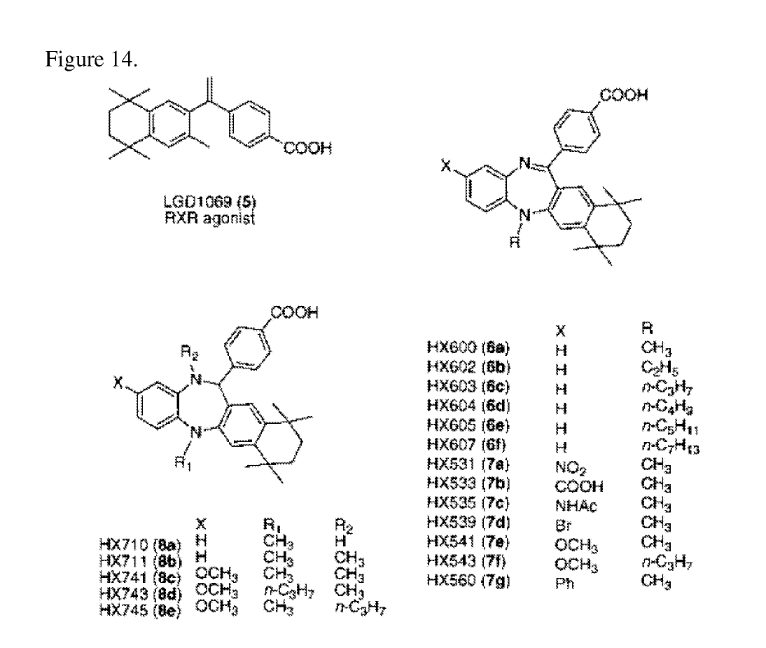

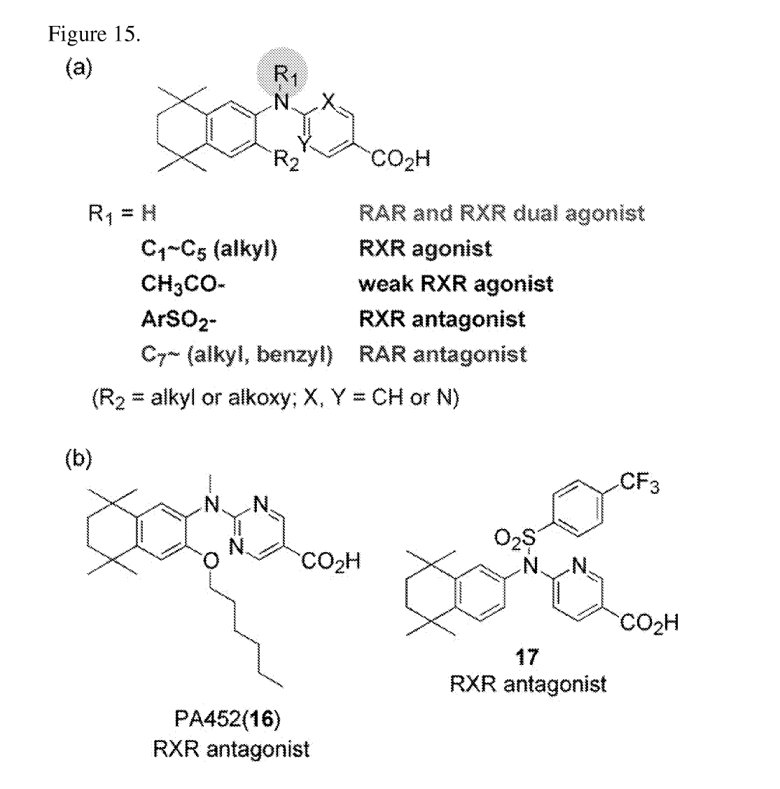

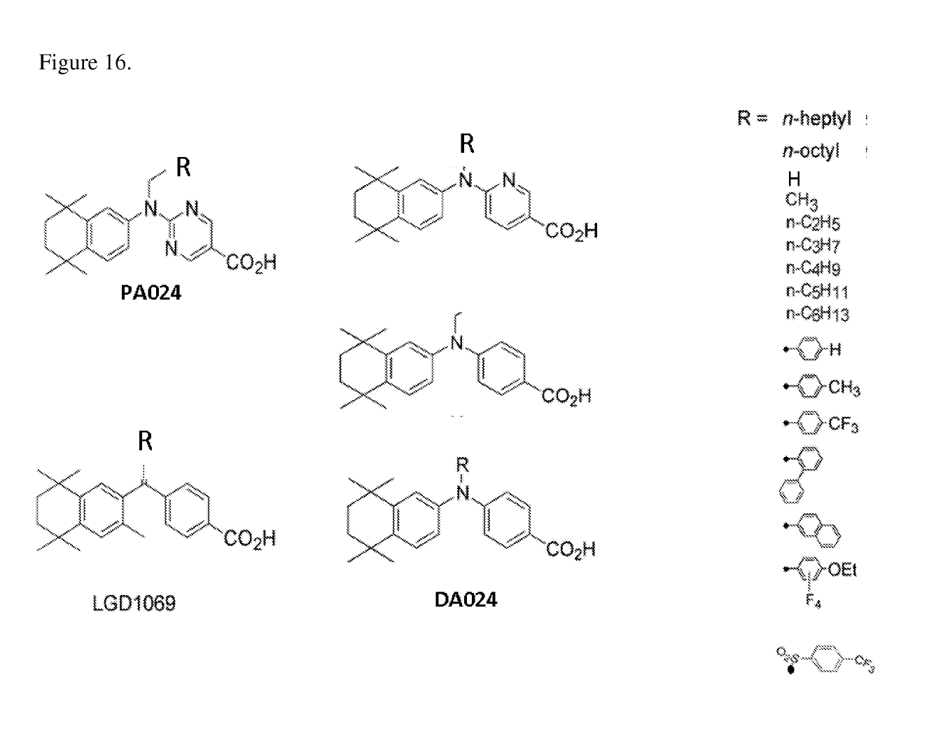

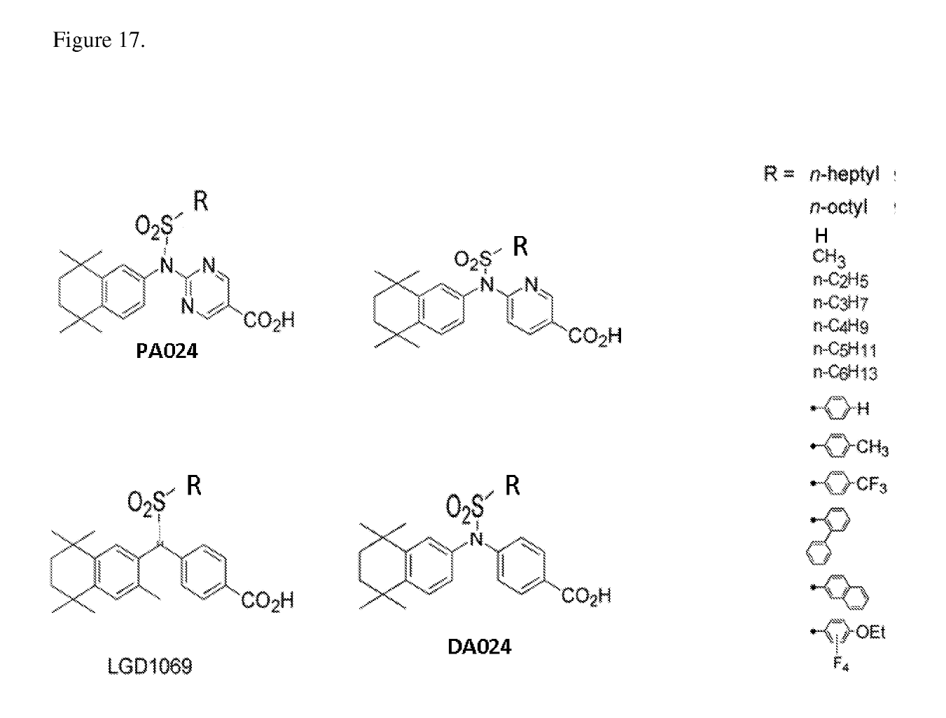

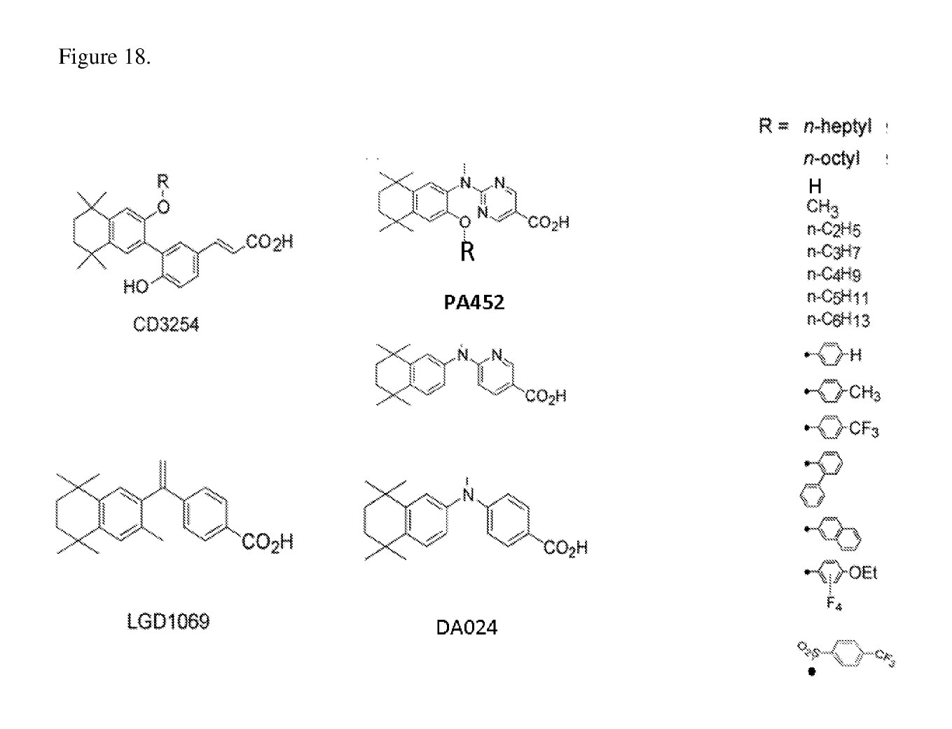

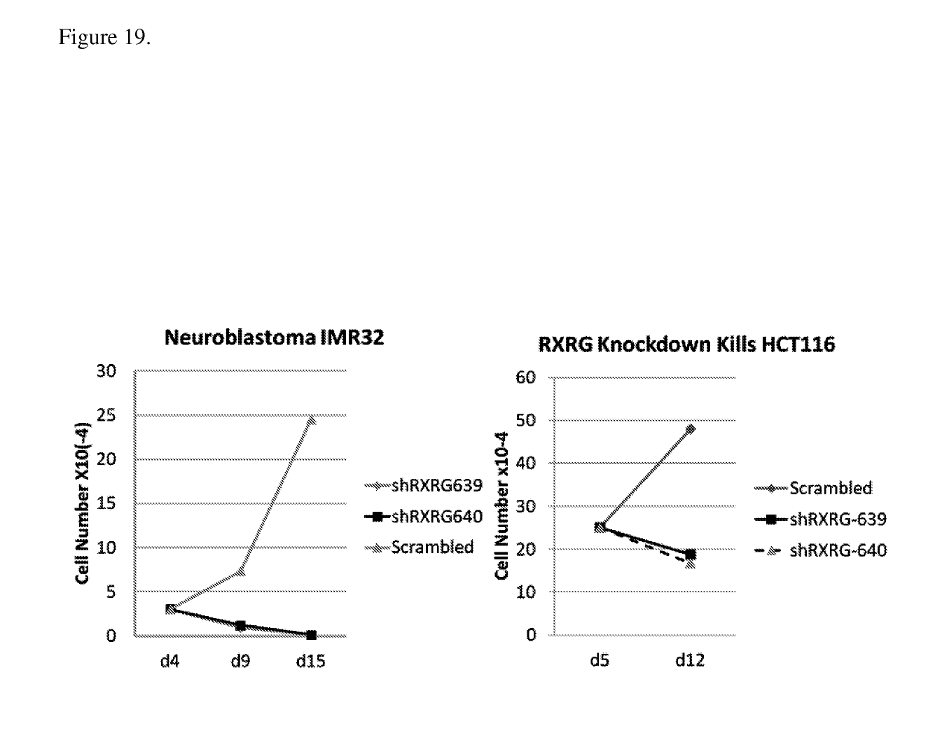

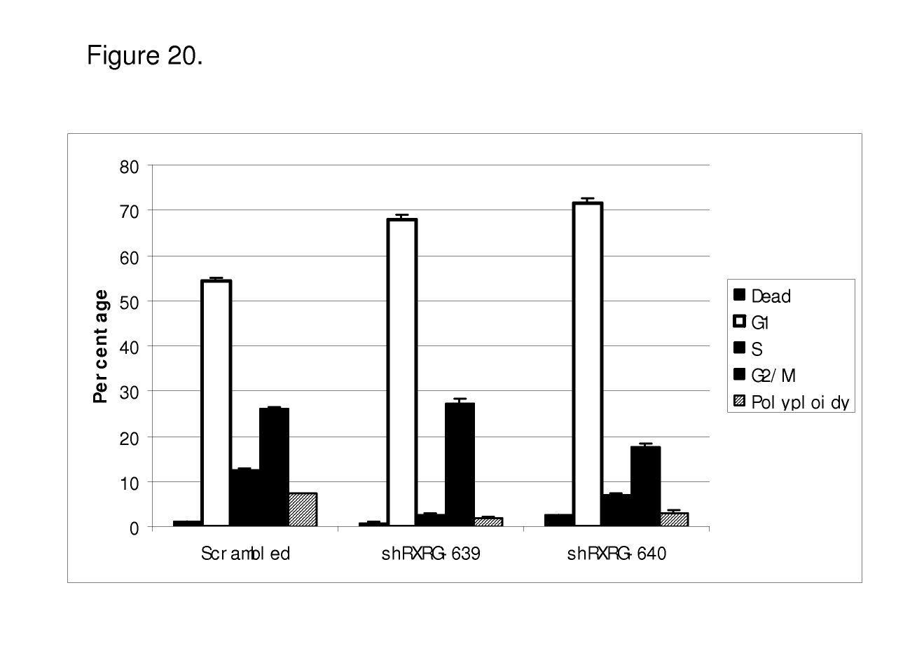

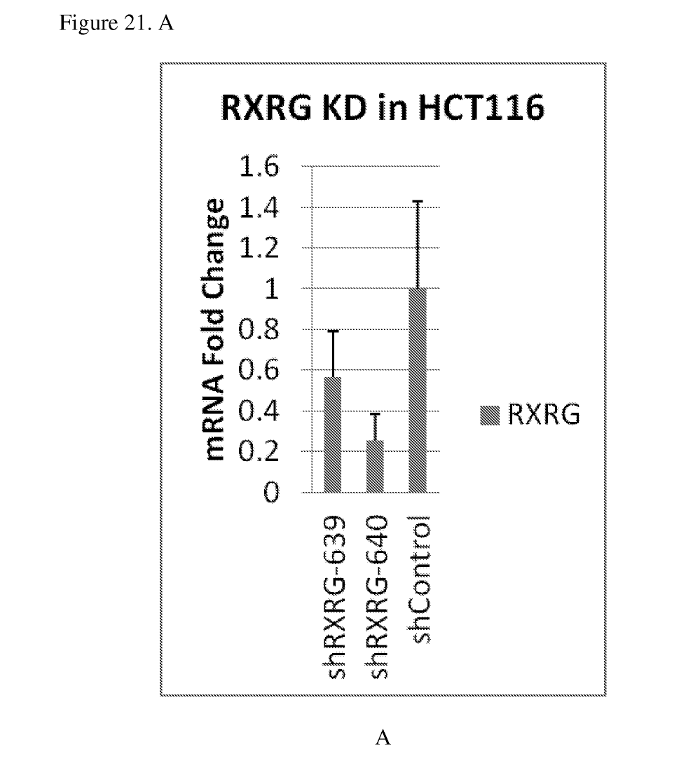

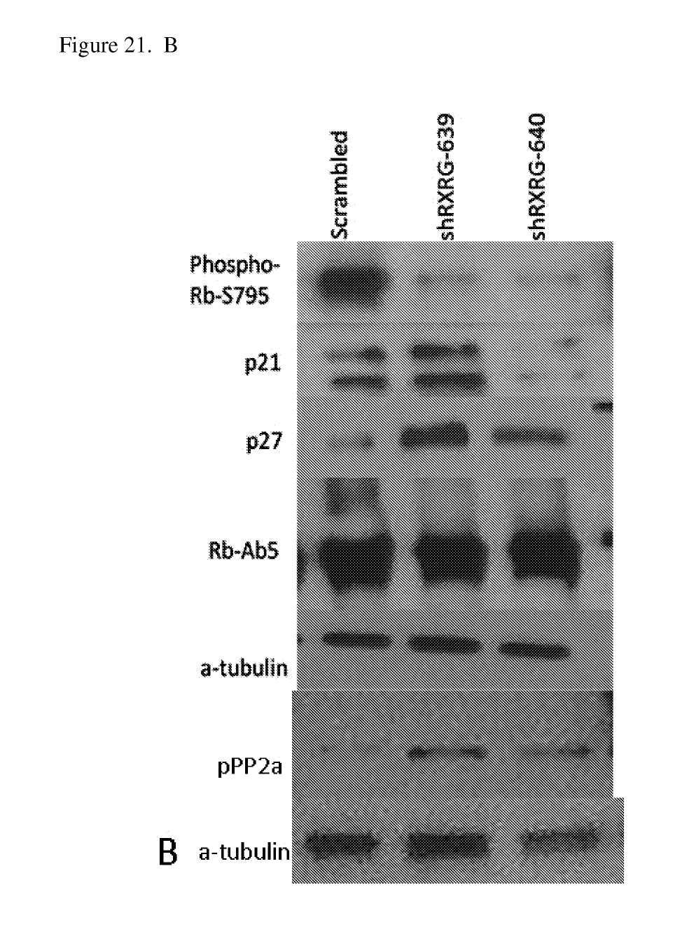

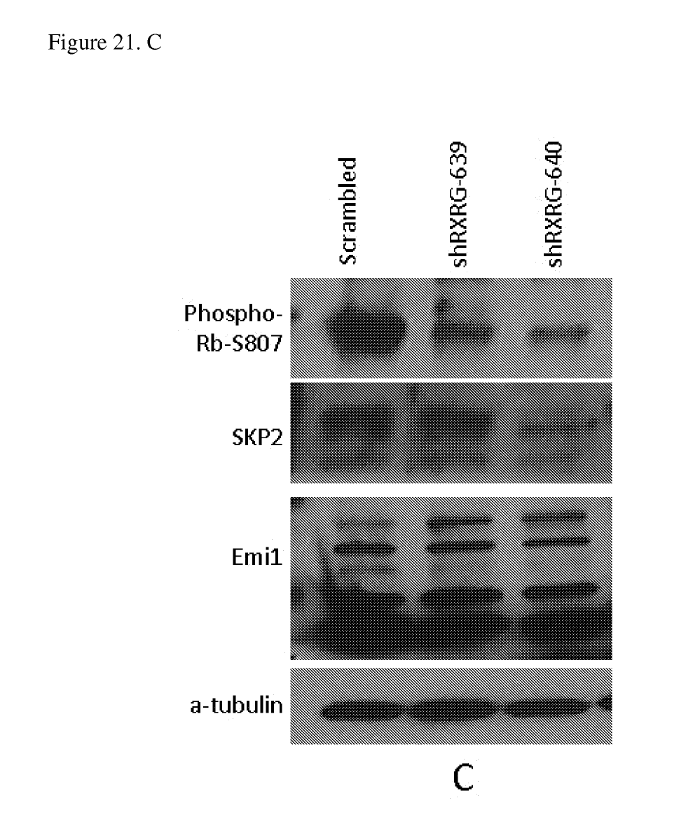

Our results showed that lentivirus-mediated RB1-Knockdown caused dissociation of the complex and Emi1 cytoplasmic translocation in HCT116 on day 5 (FIG. 11). Know-down (KD) of RXRG killed RB1+neuroblastoma IMR 32 cells and colon cancer HCT116 cells (FIG. 19). Further analysis showed that RXRG KD caused cell cycle arrest at G1 phase in KRAS mutant colon cancer cell line HCT 116 (FIG. 20). Remarkably, RXRG KD in HCT116 led to RB dephosphorylation, PP2A phosphorylation and inactivation, Emi1 hyperphosphorylation and inactivation, and SKP2 downregulation, resulting in p27 and p21 accumulation in HCT116 (FIG. 21).

### 2. RXRG Antagonist for Treating Cancer with Mutant EGRA and/or KRAS

### Examples 3

### RXRG Antagonist Suppressed Cancer Cell Growth

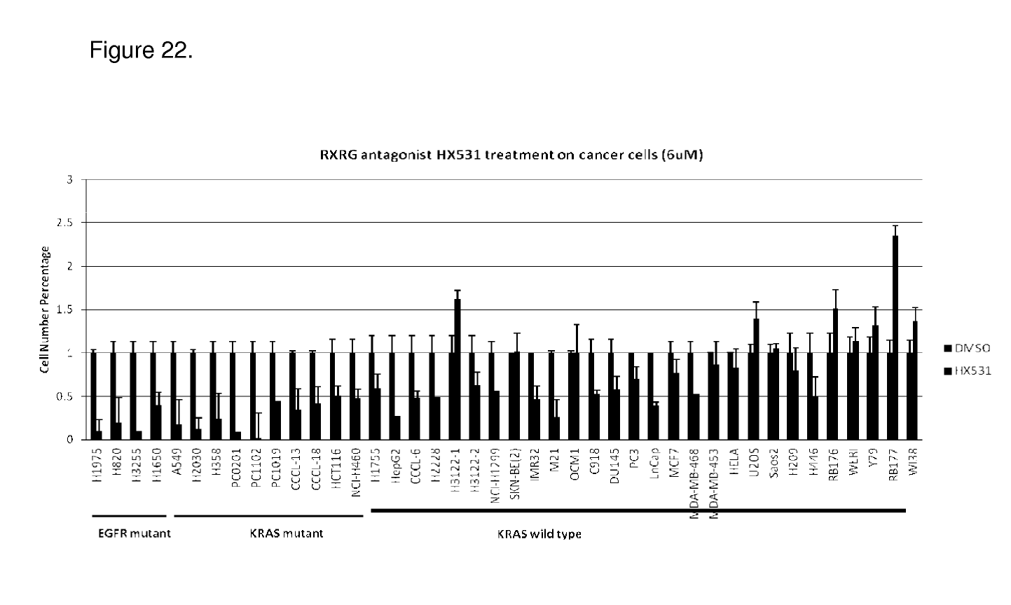

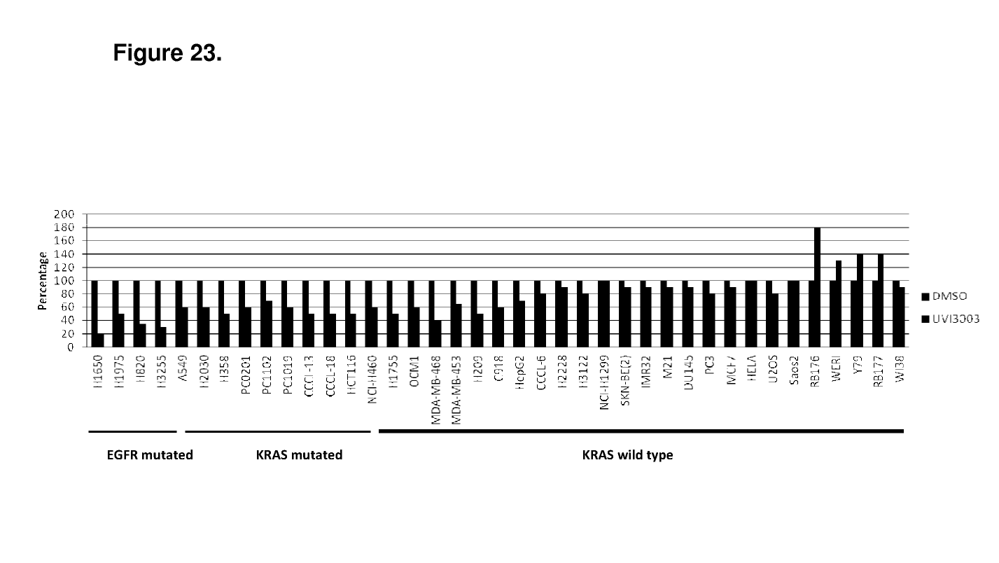

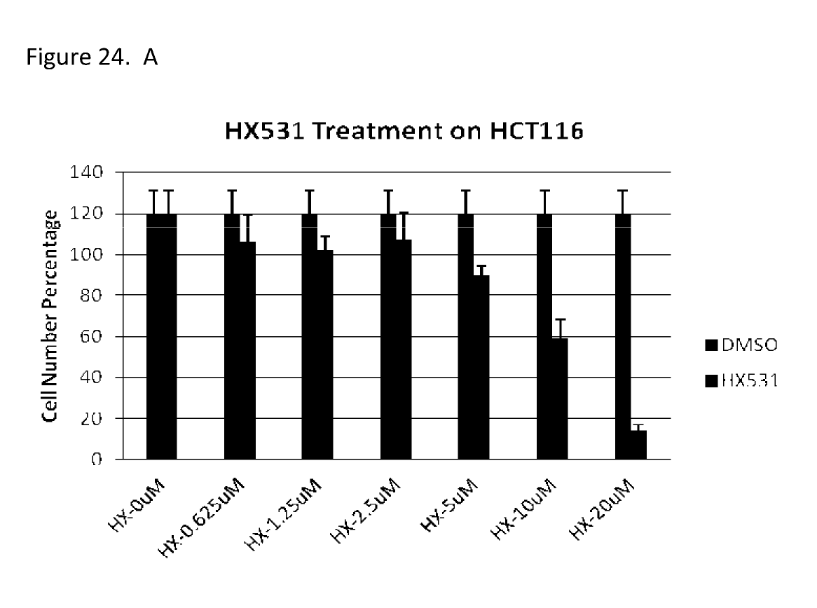

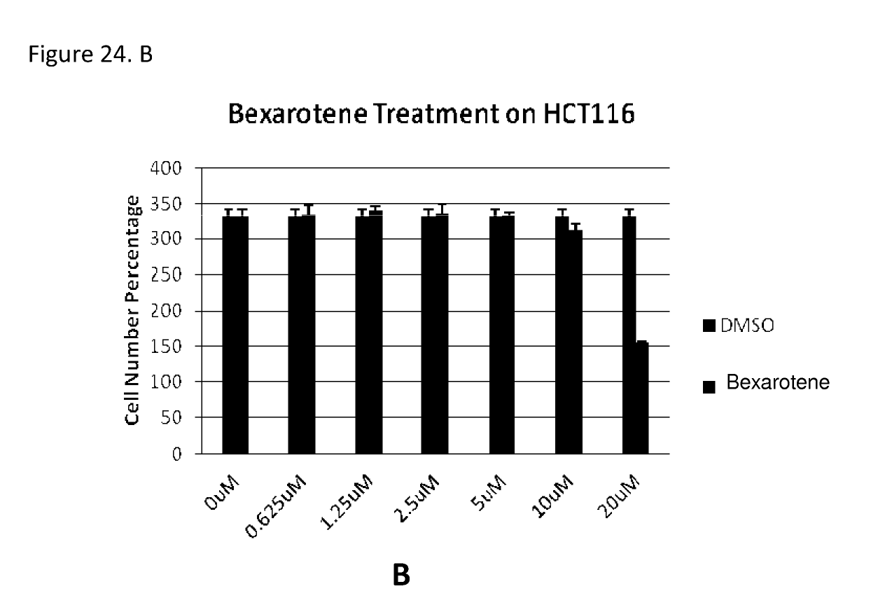

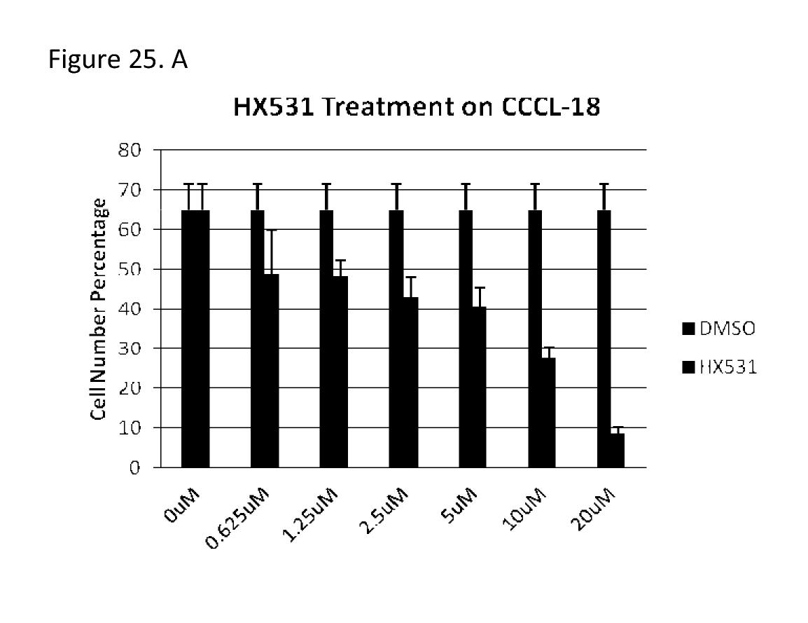

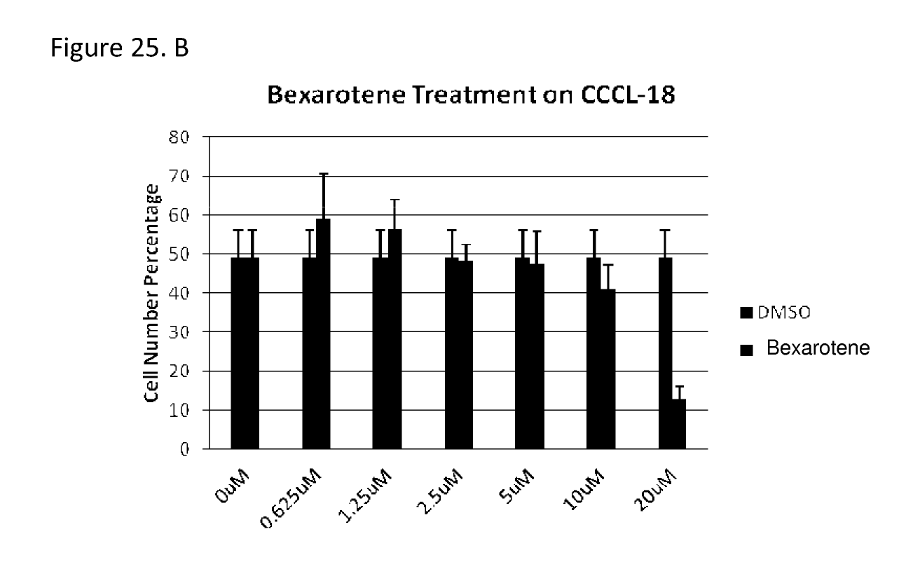

To test the effects of RXRG antagonist on cancer cells, we treated cells with HX531 using the protocols described above. EGFR and KRAS activated NSCLC, pancreatic and colon cancers were sensitive to RXR antagonist HX531 treatment. PTEN mutant prostate cancer line LnCap and PC3, and breast cancer cell line MDA-MB-468 are also sensitive to HX531 treatment. RB1 mutated retinoblastoma and Saos2, and normal fibroblasts WI38, however, are not sensitive to HX531 treatment (FIG. 22). Another RXRG antagonist, UVI3003 gave similar results (FIG. 23). Our tests showed the inhibition of HCT116 growth by HX531 is dose-dependent, as illustrated in FIG. 24. RXRG agonist, Bexarotene, on the other hand, does not show inhibition. Instead, at low concentration it slightly promotes the growth of HCT116, a KRAS-mutated colon cancer cell line. Test of HX531 on another type of colon cancer cell CCCL-18 produced similar results (FIG. 25).

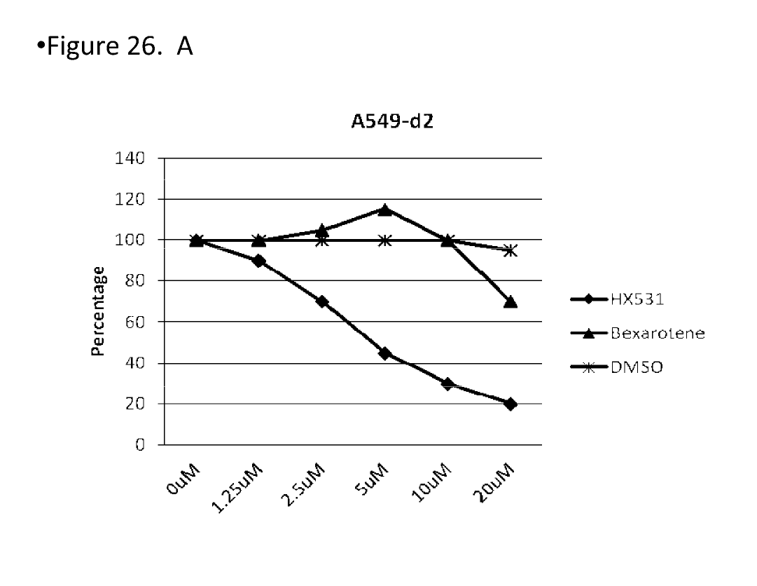

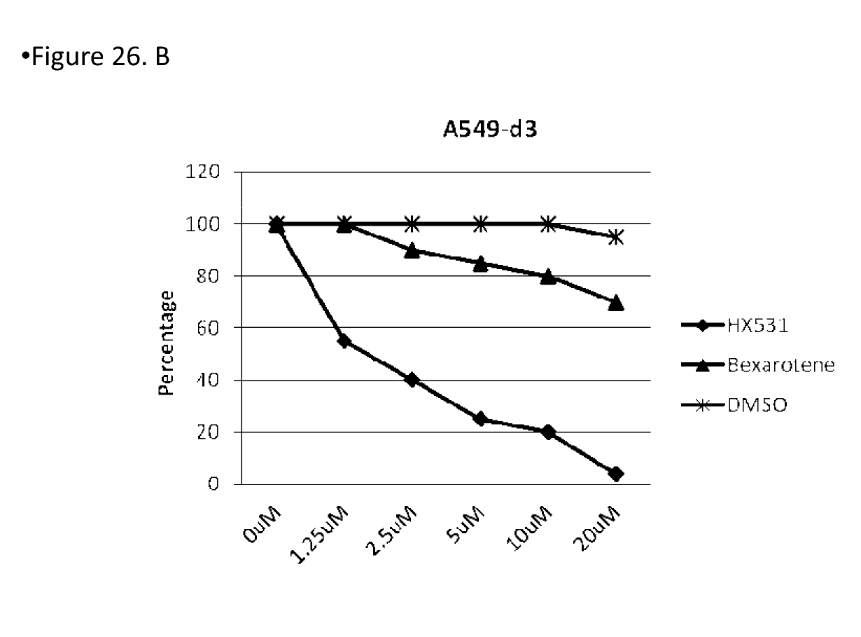

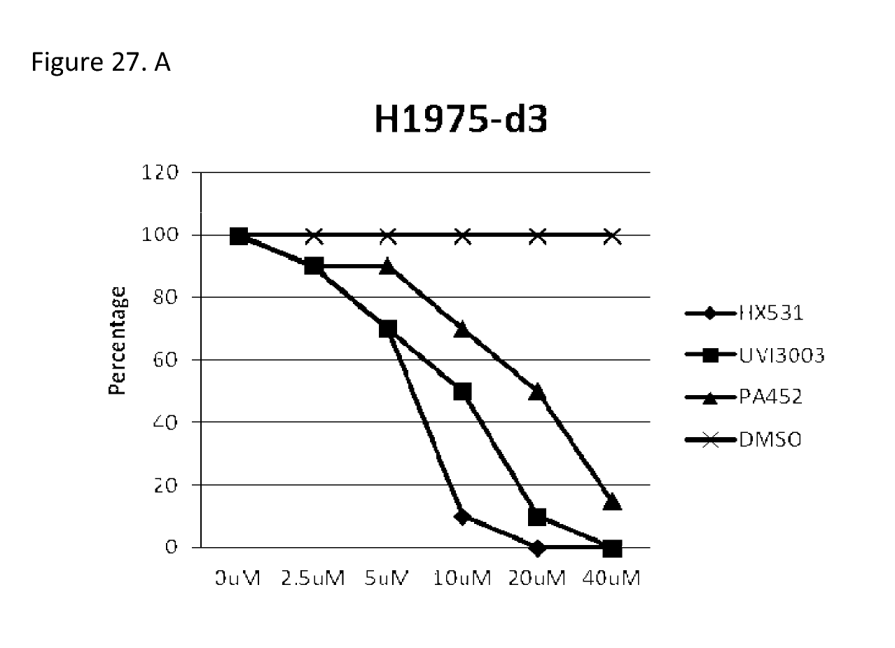

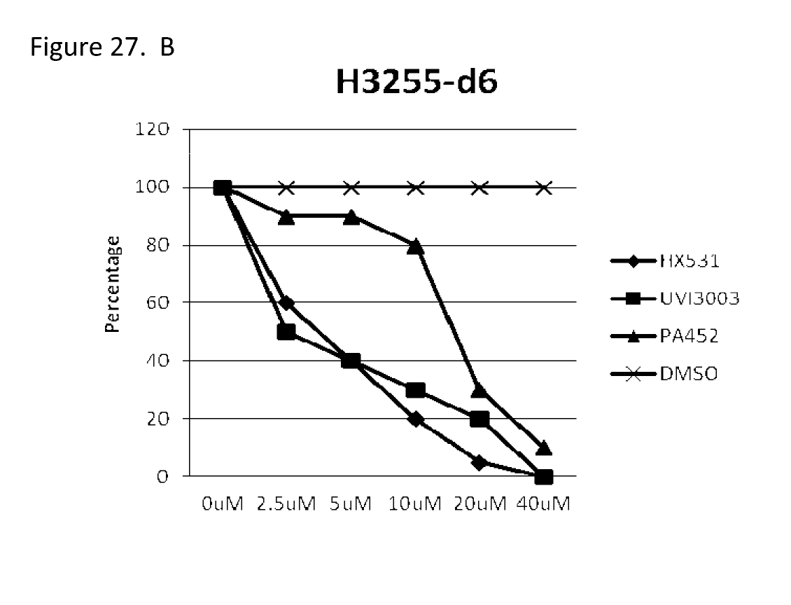

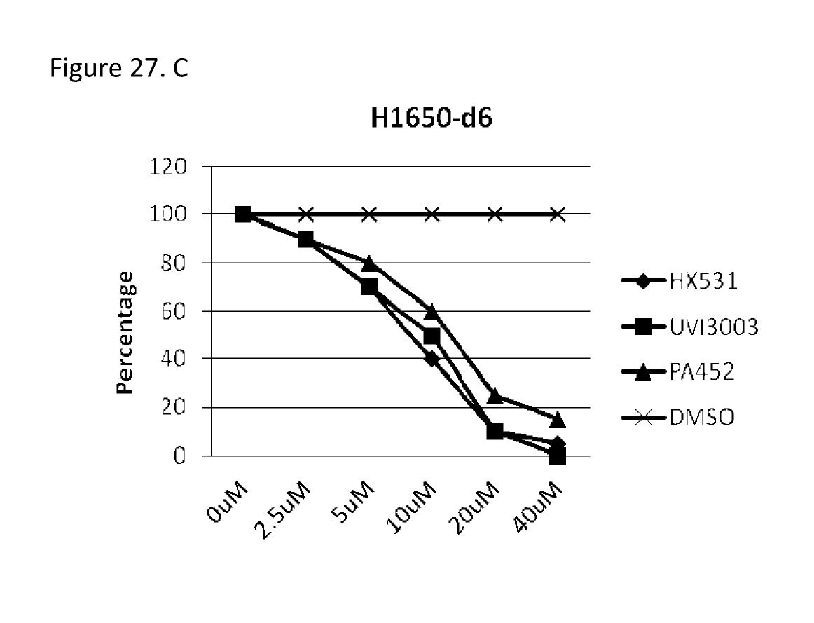

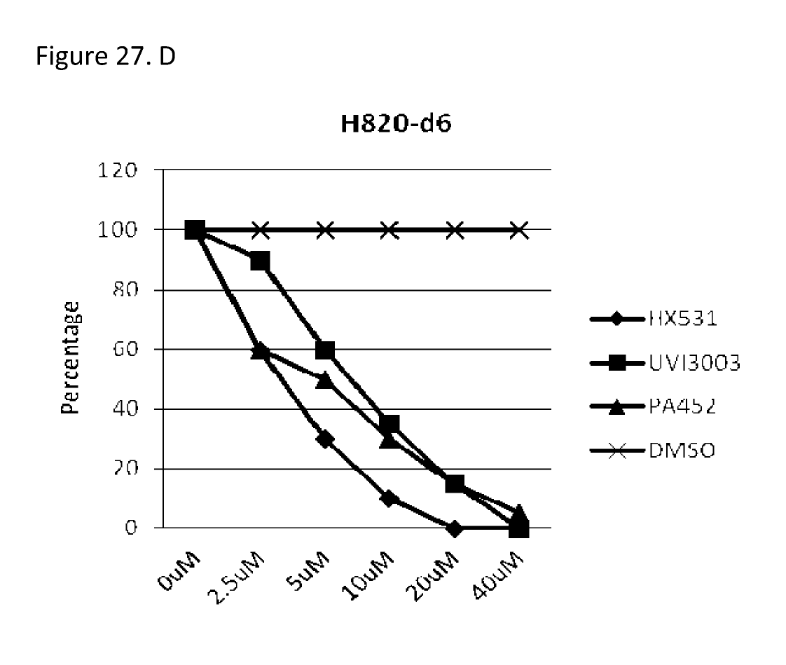

KRAS mutant non-small cell lung cancer (NSCLC) A549 is also sensitive to RXRG antagonist, HX531, treatment (FIG. 26). We test more RXRG antagonists on EGFR mutant lung cancer cell lines (H1975, H3225, H1650 and H820). All cells proved to be sensitive to tested RXRG antagonists (HX531, UVI3003 and PA452, FIG. 27).

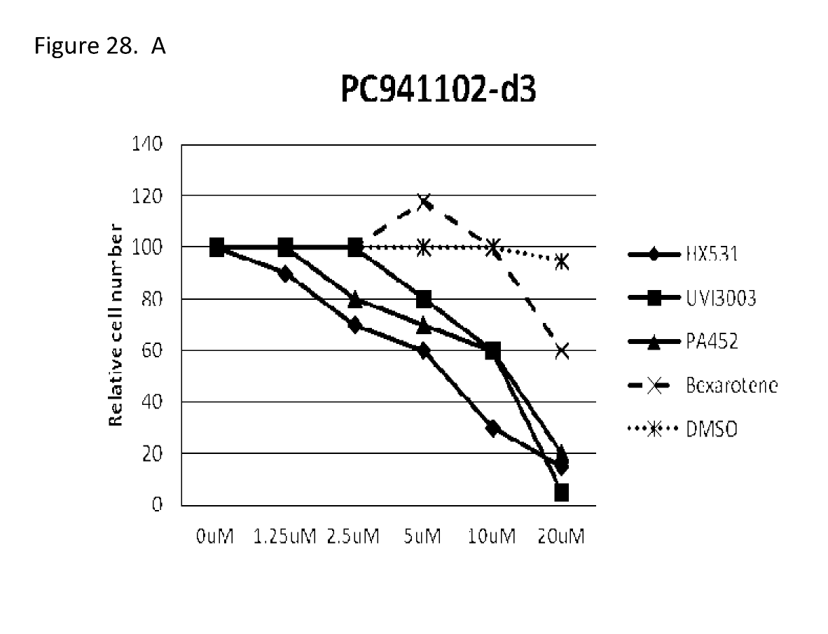

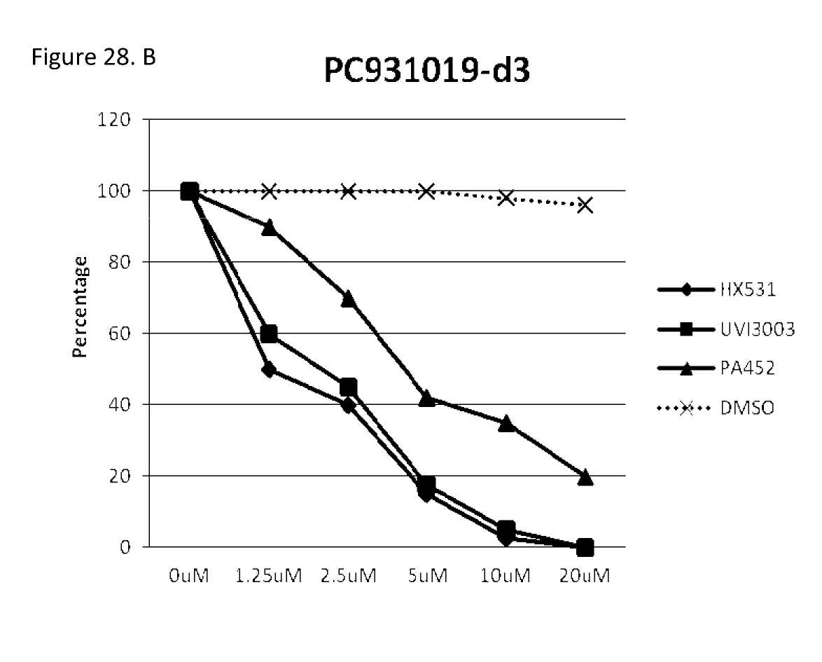

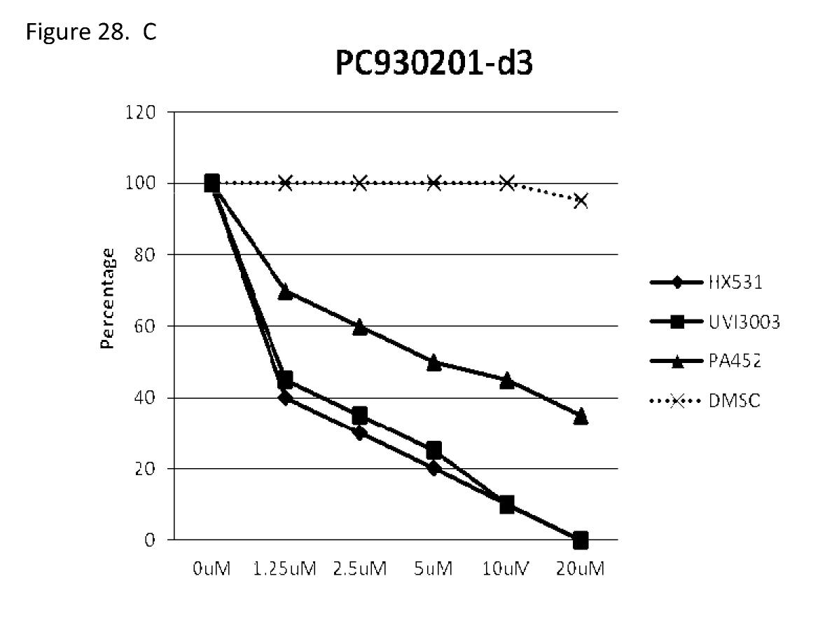

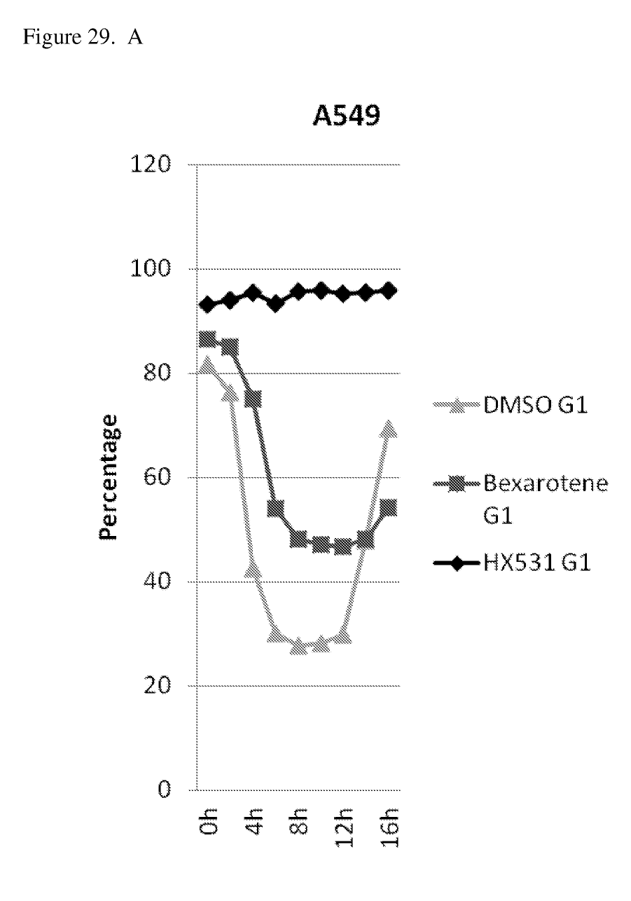

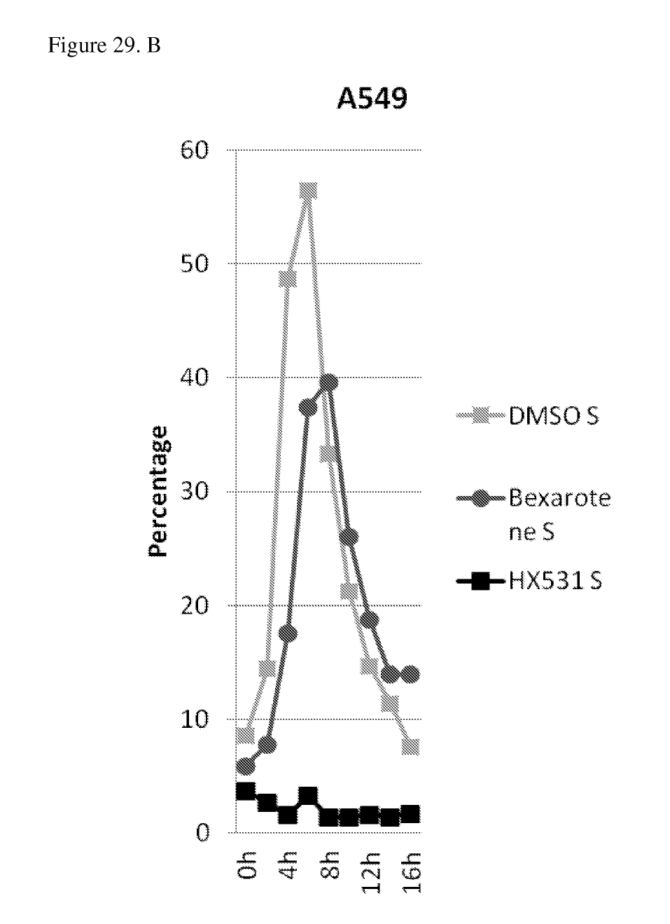

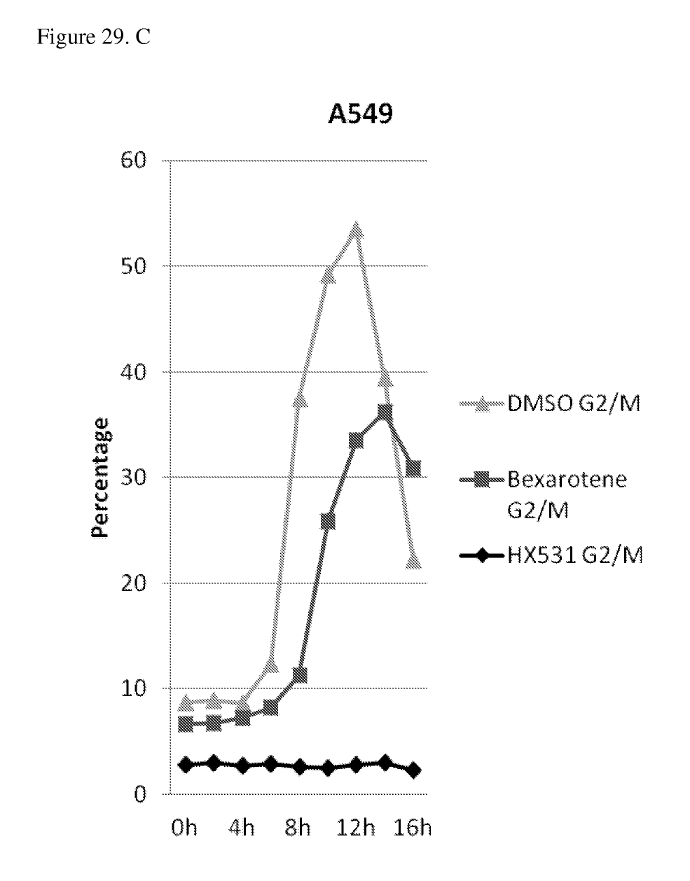

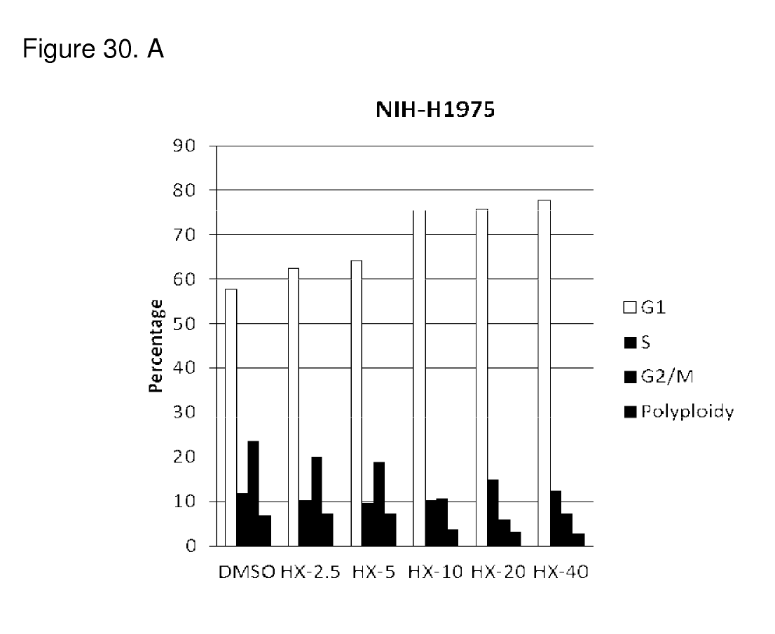

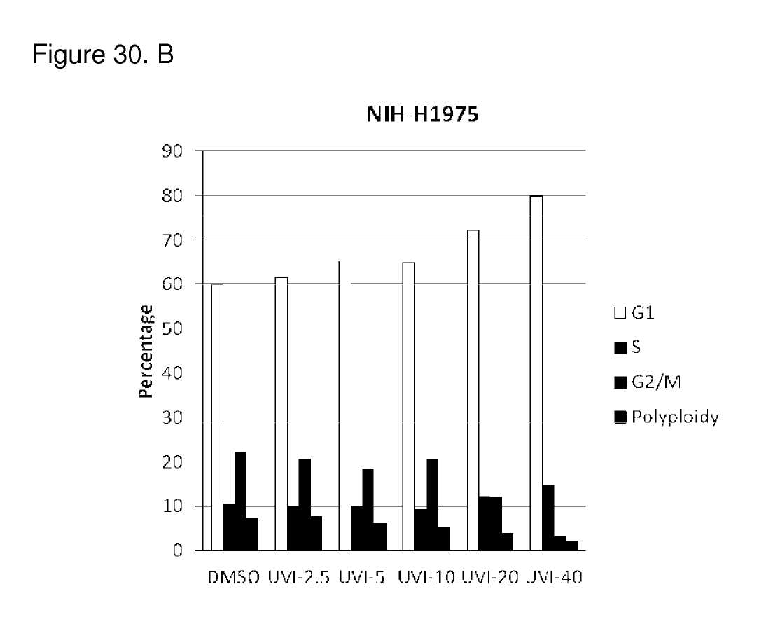

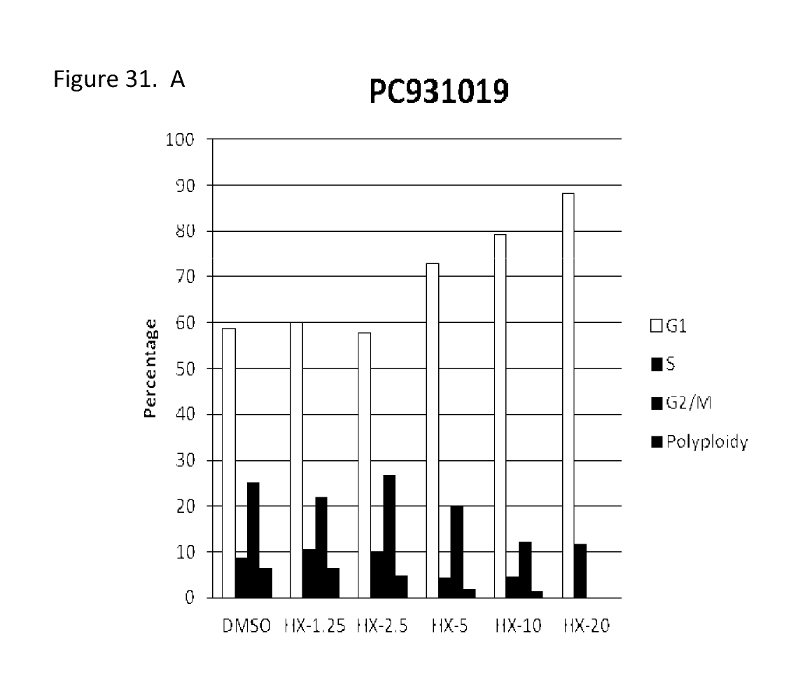

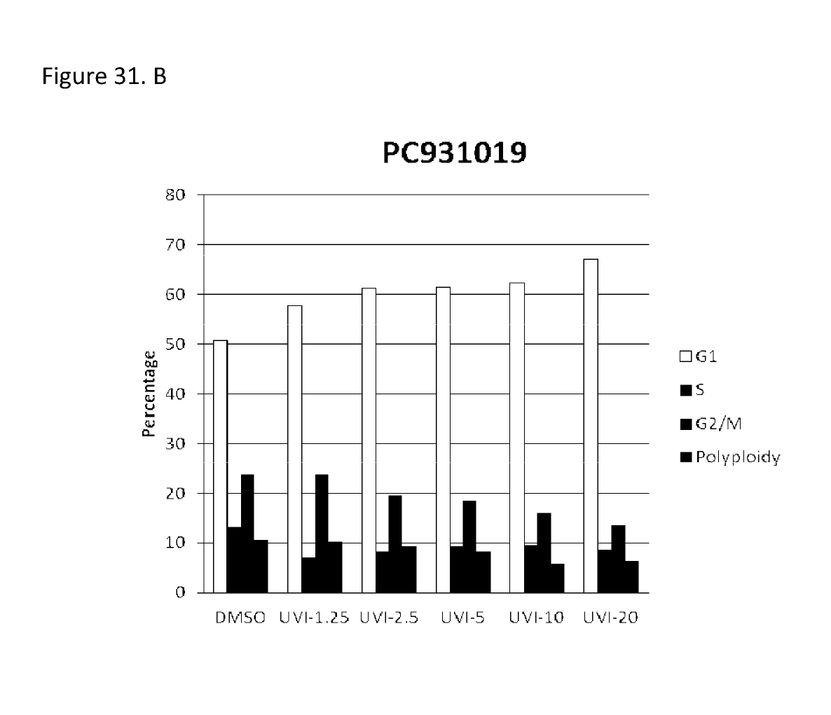

We next tested RXRG antagonists on KRAS mutated pancreatic cancer cells, including PC941102, PC931019, and PC930201. All were sensitive to the test RXRG antagonist (HX531, UVI3003 and PA452, FIG. 28). Cell cycle analysis showed G1 arrest after HX531 treatment in lung cancer A549 cells for 2 days. (FIG. 29). G 1-S transition in EGFR mutant NSCLC cancer cell H1975 was inhibited by HX531 and UVI3003 on day 3 (FIG. 30). KRAS mutant pancreatic cancer cell PC931919 is similarly sensitive to RXRG antagonist HX531 and UVI3003 (FIG. 31).

### Example 4

### RXRG Antagonist LED to SPC Dissociation

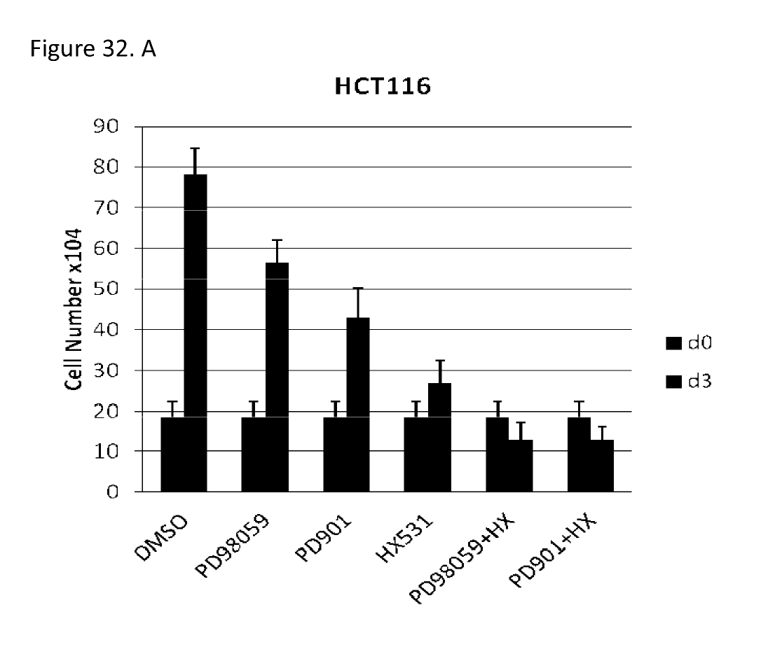

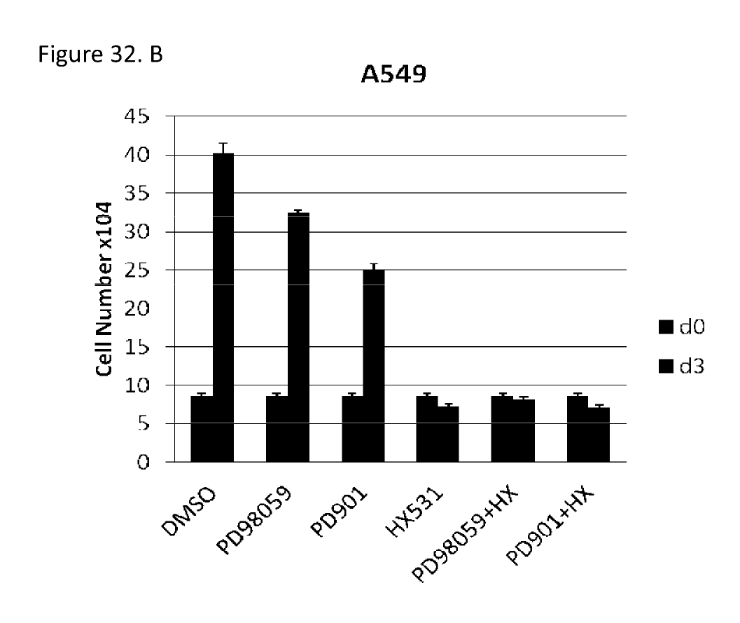

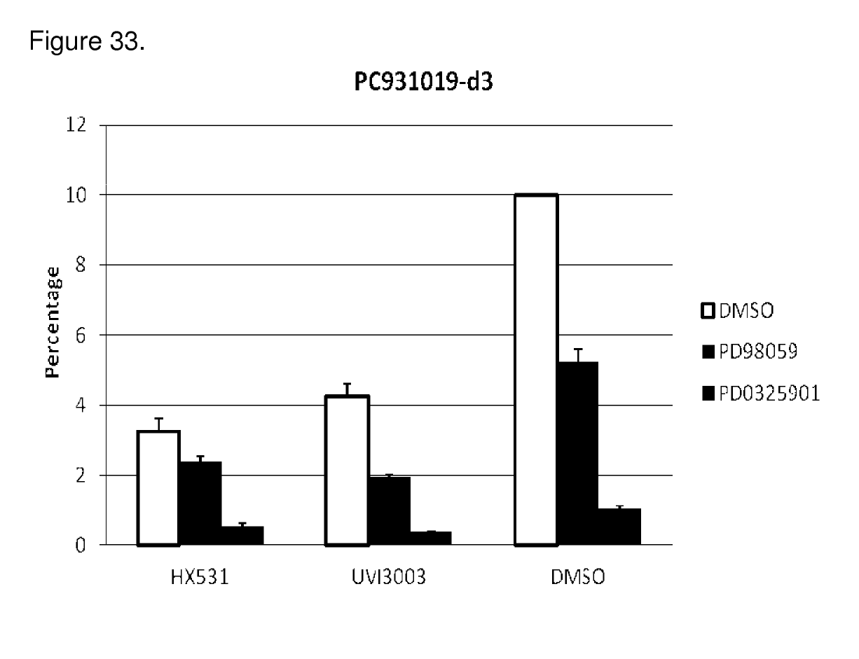

We further tested whether there were synergistic effects between HX531 and MEK inhibitors. The two MEK inhibitors tested, PD98059 and PD0325901 both showed synergistic effects with HX531 toward colon cancer HCT 116 and A549 (FIG. 32). The same synergistic effects were detected for pancreatic cancer (FIG. 33).

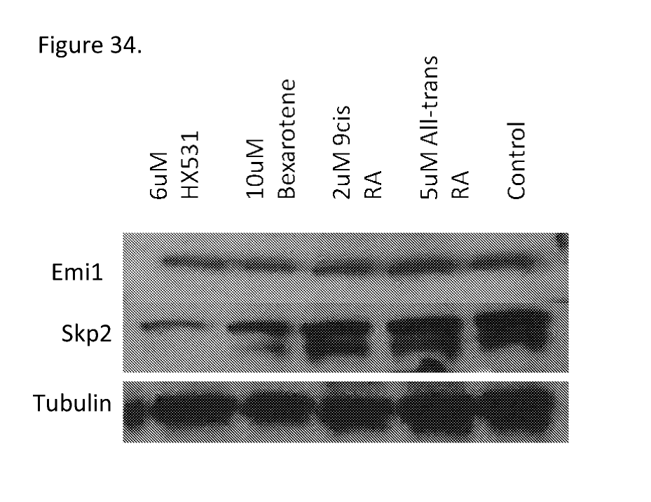

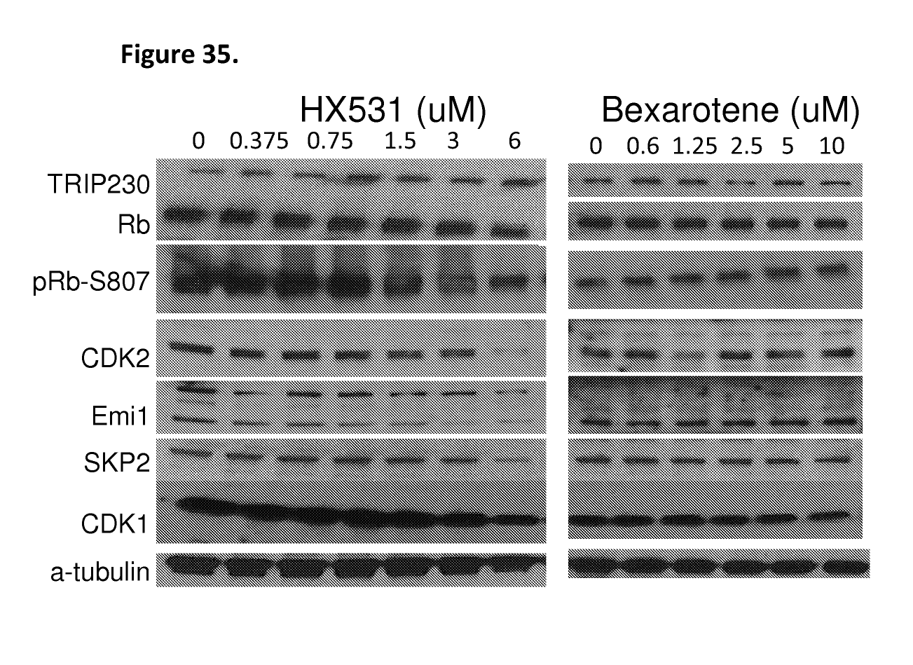

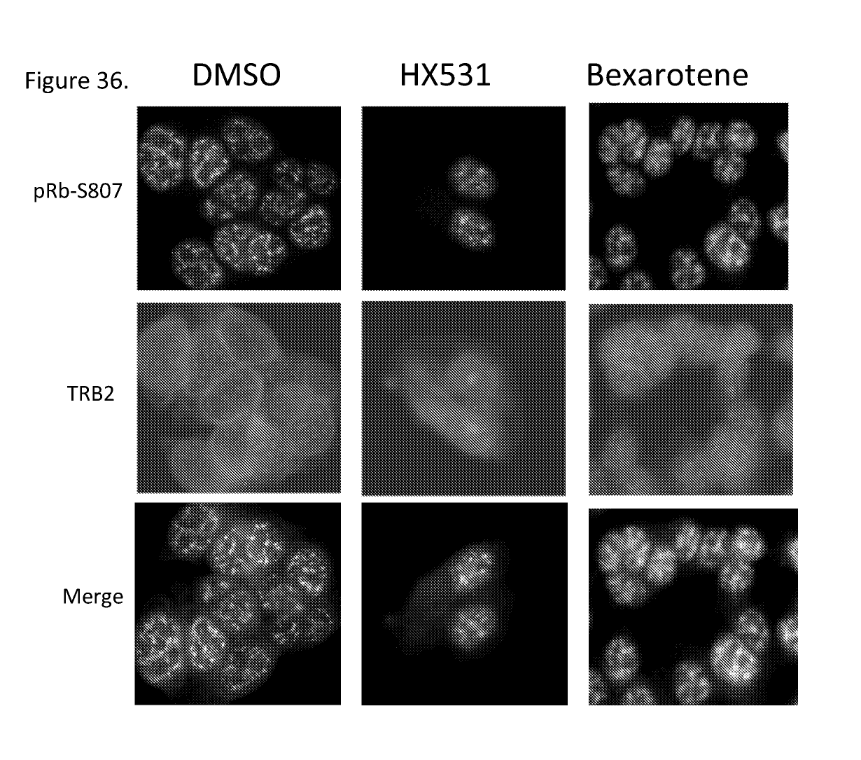

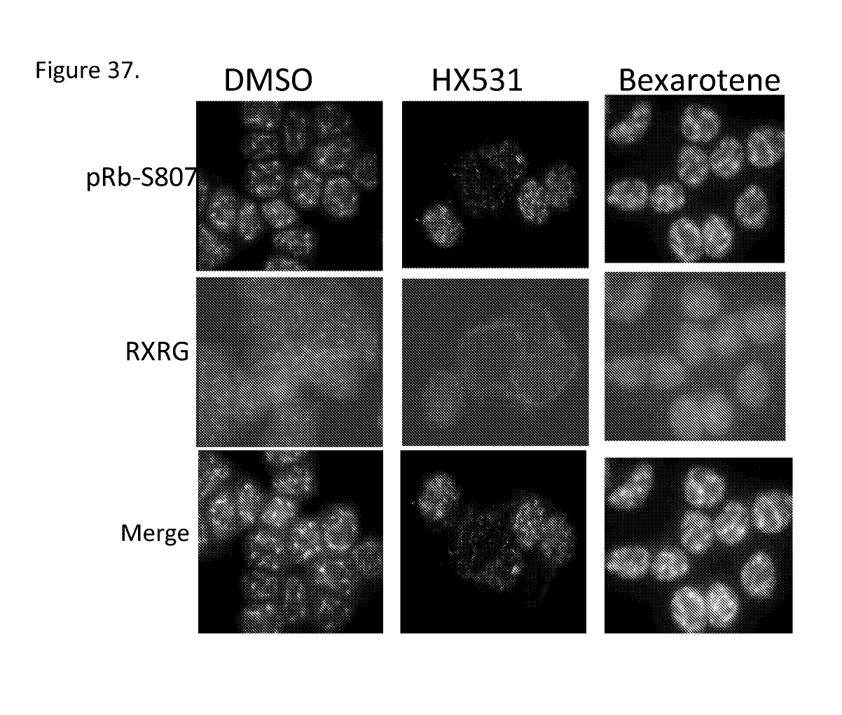

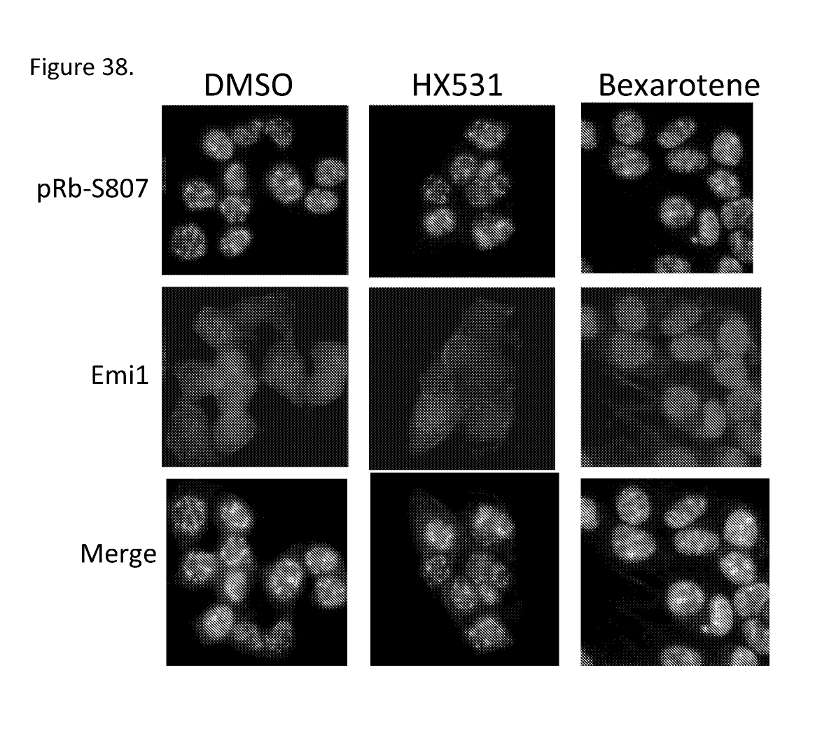

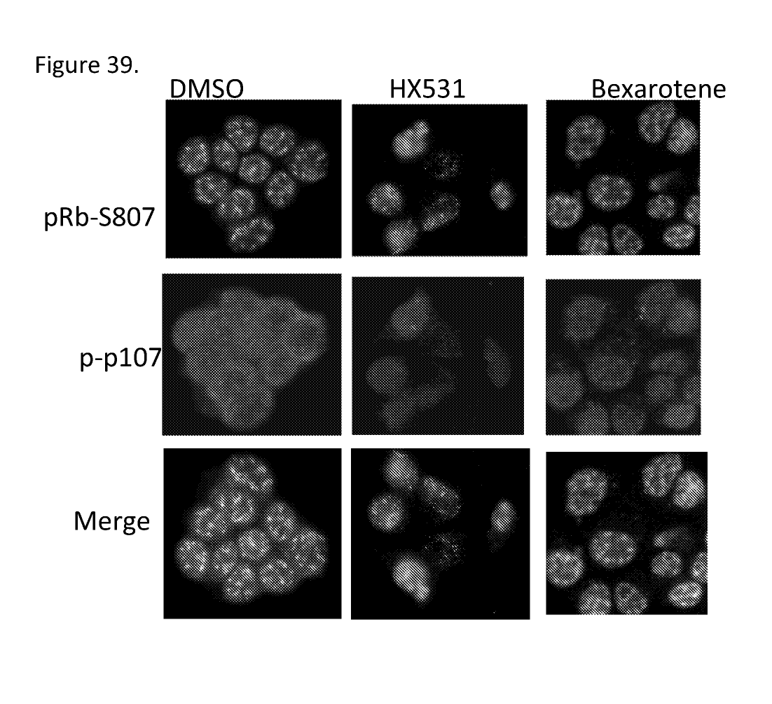

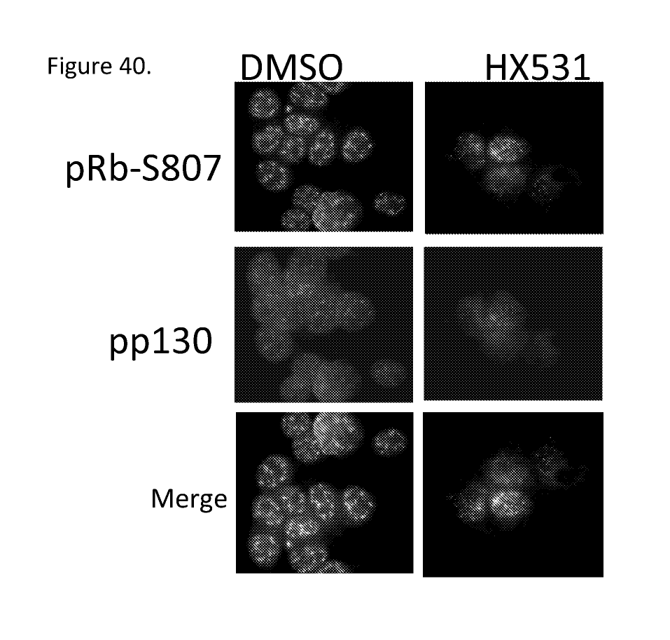

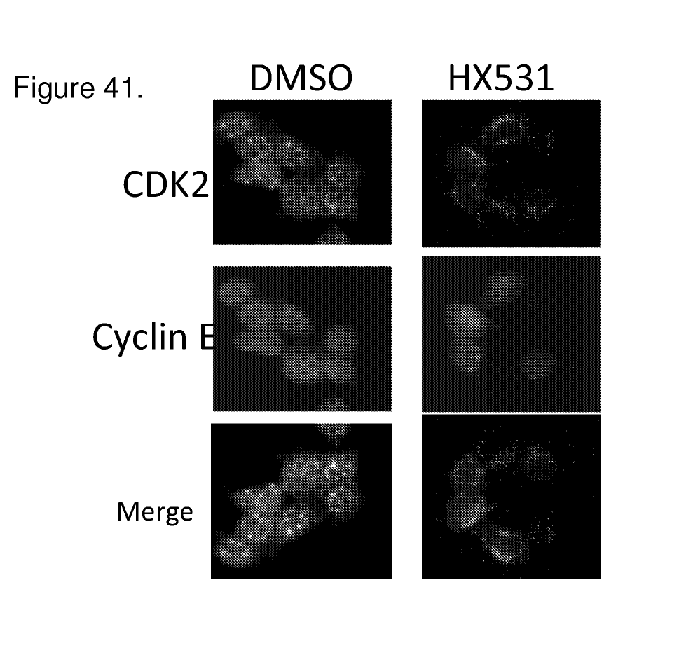

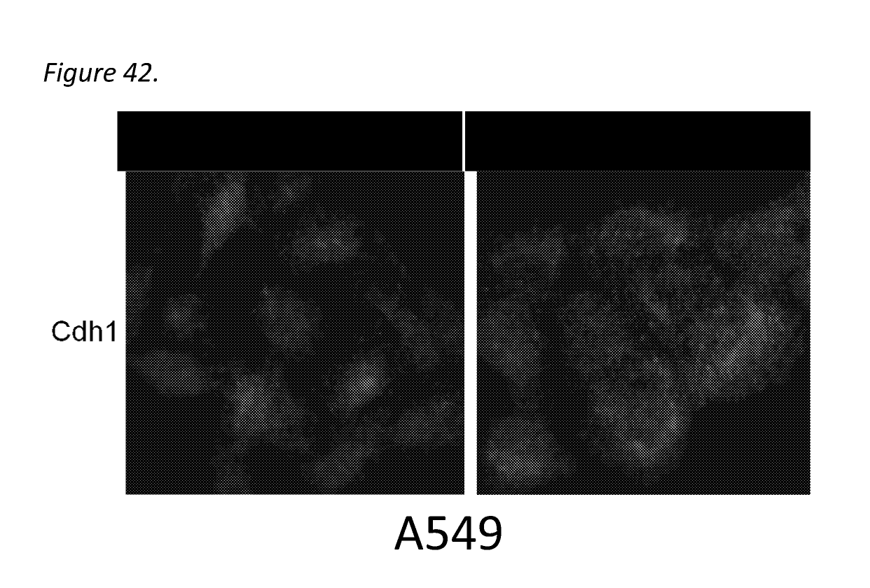

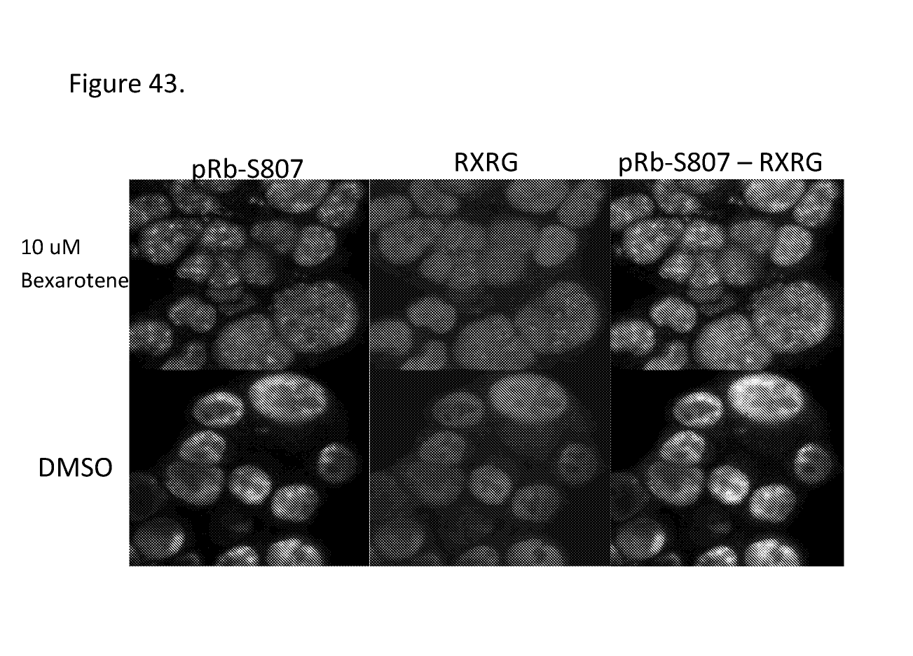

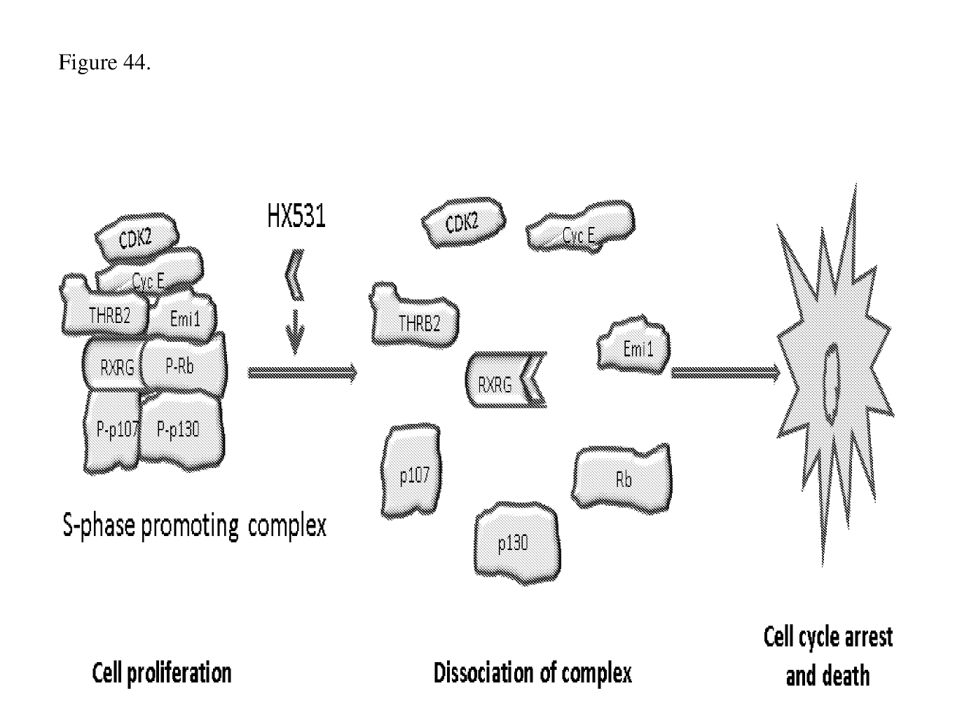

Western blot analysis showed that HX531 treatment caused significant Emi1 downregulation and inactivation of Emi1. SKP2 degradation was also observed in HCT116 on day 2 (FIG. 34). RB de-phosphorylation and downregulation of CDK2, Emi1, and SKP2 in HCT 116 on Day 2 of treatment (FIG. 35). Immunofluorescence results further showed the dissociation of the S-phase promoting complex by RXR antagonist HX531 (FIGS. 36-41). Treatment of HX531 also led to Cdh1 nuclear translocation in lung cancer cells A549 (FIG. 42). Different than RXRG antagonist, RXRG agonist, Bexarotene promoted S-phase promoting complex formation and pRB-RXRG interaction in HCT 116 (FIG. 43). Without wishing to be bound by any theory, we propose that RXRG antagonists can lead to the dissociation of the S-phase promoting complex, which in turn lead to cell cycle arrest and cell death, achieving the goal of inhibiting cancer cell growth (FIG. 44).

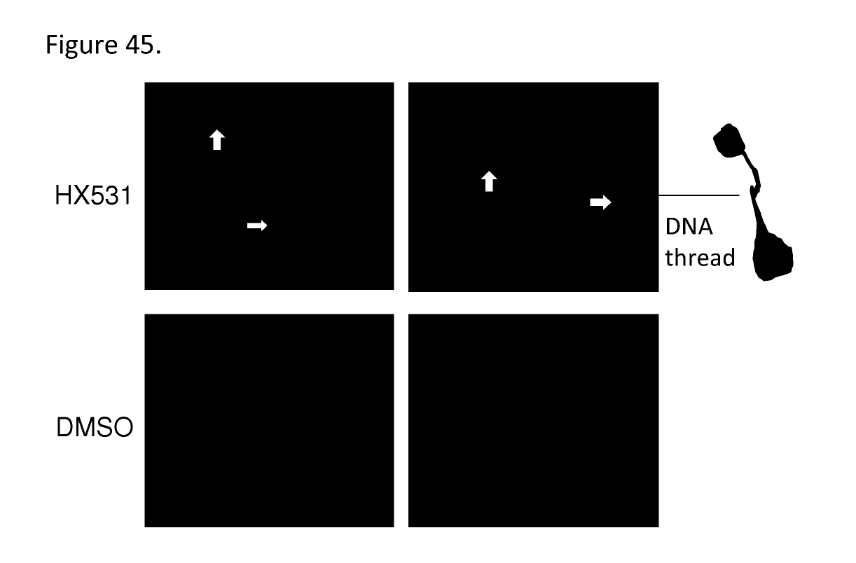

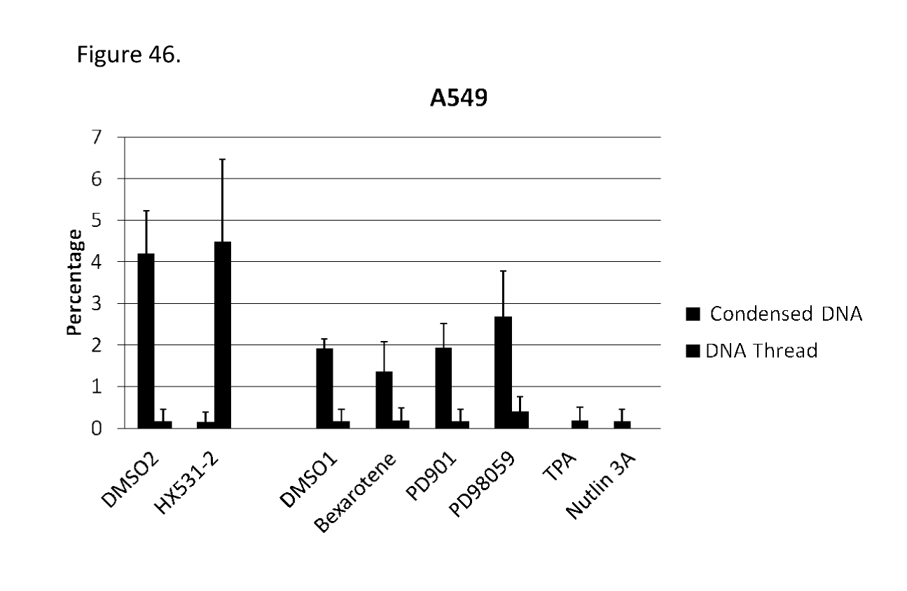

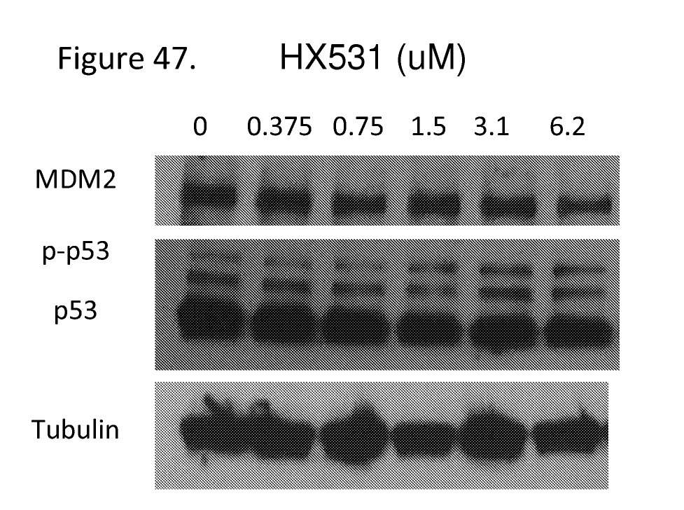

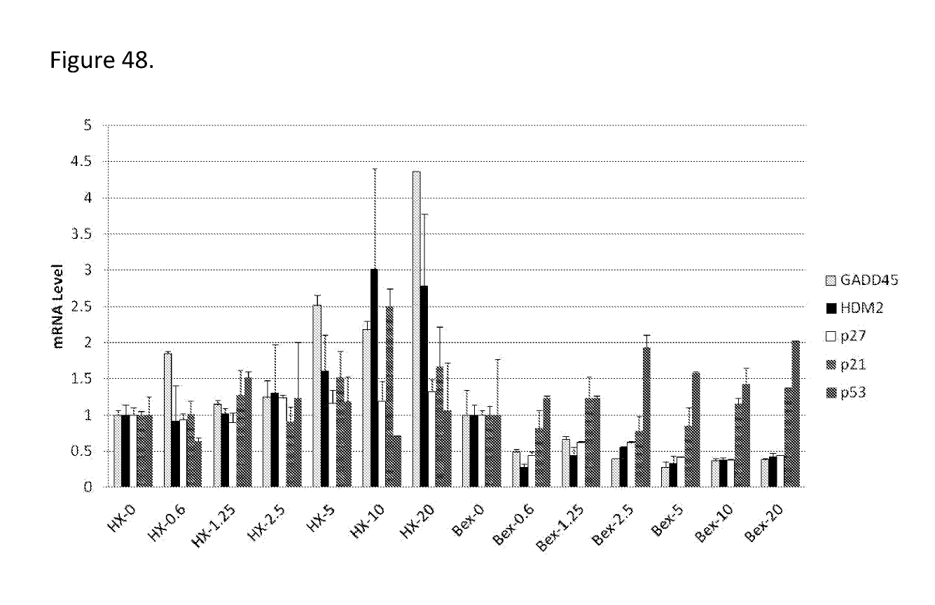

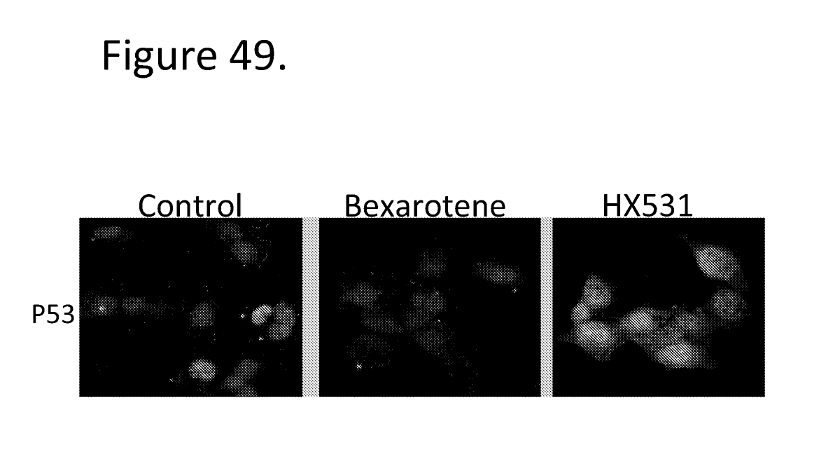

Our data further showed that HX531 treatment of A549 cells caused DNA condensation and separation defects (FIGS. 45-46). MDM2 downregulation and inactivation, and p53 phosphorylation and activation were also observed upon treatment of A549 cells with H531 for two days.

### Example 5

### RXRG Antagonist Treatment Up-Regulated p53 Target Genes

Real Time PCR Analysis:

Total RNA was isolated from puromycin-selected cells using RNeasy Mini Kit (Qiagen) or GenElute™ Mammalian Total RNA Miniprep Kit (Sigma). RNA reverse transcription was performed with ImProm-II™ Reverse Transcription System (Promega). Primers were designed by Beacon Designer software (Premier Biosoft International) or web-based Primer3 (http://frodo.wi.mit.edu/primer3/). Relative mRNA levels were determined by qPCR using QuantiTect SYBR Green PCR Kit (Qiagen) or Fermentus Maxima® SYBR Green qPCR Master Mix on an Applied Biosystems ABI 7900HT Sequence Detection System. Two samples for each group were collected and evaluated in triplicate and normalized to β-actin mRNA quantitated in parallel. Program conditions: activation, 95° C. 10 min; amplification, 40 cycles (denaturation 95° C. 20 sec, annealing 54° C. 30 sec, extension 72° C. 30 sec). qPCR primers are listed in Table 7.

TABLE 7
Sequences of qPCR primers.

| Primers | Sense | Antisense |
|---|---|---|
| HDM2 | ggtgaggagcaggcaaatgtg | gctggaatctgtgaggtggttac |
| GADD45A | cgaggacgacgacgaagatg | cgcaggatgttgatgtcgtt |
| SKP2 | ctagcgtctgatgagtctctatgg | gggcagcggaaggcaatc |
| RXRG | agcgatgaccactcttgttag | tcgtcagttcatgttcctctc |
| CDKN1A(p21) | cccctttcctggacactcag | caccctgcccaaccttagag |
| CDKN1B(p27) | gctccggctaactctgagga | aagaatcgtcggttgcaggt |
| RBI | ccagtaccaaagttgataatgc | ccaagaaacttttagcaccaatgcag |
| 14-3-3 | gagccatggagagagccagt | agagcaggtttcgctcttcg |
| β-Actin | gcaagcaggagtatgacgagtc | caagaaagggtgtaacgcaactaag |

A549 and HCT116 cells were treated with RXRG antagonist HX531 or agonist Bexarotene. On Day 2, total RNA was isolated. After reverse transcription, relative mRNA levels were determined by real time PCR as described above. p53 targeted genes, such as GADD45, HDM2, p27, and p21 were up-regulated leading to apoptosis. Immunofluorescence analysis showed activated p53 in A549 on day 2.

Different from RXRG antagonist HX531, RXRG against Bexarotene did not caused increased level of GADD45, HDM2, and p27.

### Example 6

### RXRG Antagonist Suppressed Lung Cancer in Mice

Intravenous Tail Vein Injection for Lung Cancer—RXRG Antagonist Suppressed Lung Cancer Formation:

The mouse is carefully warmed (e.g. with a heat lamp) to cause venodilation, increasing ease of vascular access. The mouse is placed in a restraining device such that the lateral tail veins are accessible. The tail is cleansed with a sterile alcohol wipe prior to injection. A 0.5″ 25 gauge or larger gauge needle is directed into a lateral tail vein, bevel up, at an angle of approximately 20°, preferably midway down the tail. Once the vein has been penetrated, the needle is directed cranially a distance of approximately 2 mm. The cell suspension to be injected (no more than 0.5 mL) is slowly administered, making sure that no swelling is detected cranial to the injection site. Pressure is applied over the injection site after the needle is withdrawn from the vein for approximately 30 seconds with gauze (or similar material) to prevent hematoma formation and make sure that hemostasis is achieved. 1 to 2 million cells are injected for each mouse, in some cases 6-week-old male athymic (nude) mice.

Effectiveness on NSCLC xenograft is tested at different dosages, for example, 16.7 μg/g/d HX531 (99.7 μg/ml in drinking water suspension, according to mouse can drink 15% water each day, to reach 10-20 μM in mouse body), 15 μg/g UVI3003 (89.9 μg/ml in drinking water), 15 μg/g C43 (89.9 μg/ml in drinking water), 12 μg/g/d bexarotene (71.86 μg/ml in drinking water) and control (DMSO) for 1 month. KRAS (A549) and EGFR mutant (H1650) NSCLC lines have multiple (for example, 10) groups. Each group needs several nude mice; in some cases, 12.

Before xenograft, the NSCLC lines A549 and H1650 can be labeled with lentiviral luciferase expression and selected by hygromycin. Tumor formation is monitored by luciferase imaging. Mice health is checked every two days. The mice are sacrificed by CO₂ or cardiac perfusion if they are obviously sick, and lungs, brains, and other organs will be extracted. The weight of the lungs and tumor mass are measured and samples will are embedded in paraffin and immunostaining is performed on sections to test the HDM2, HDMX, p53, RXRG, TRB1, TRB2, Cyclin E, Emi1, CDC25C, SKP2, p130, P27, pMEK, and pERK expression. TUNEL assay is performed on sections to check the apoptosis after treatment. Total RNA is isolated from the tumors and qPCR is performed to check the HDM2, HDMX, SKP2, and E2F1 expression using human specific primers. Kaplan-Meiyer survival curve is generated for comparison of treatment effectiveness. Mouse blood and some mouse organs such as brain and liver is collected and frozen to check the drug concentration and metabolism.

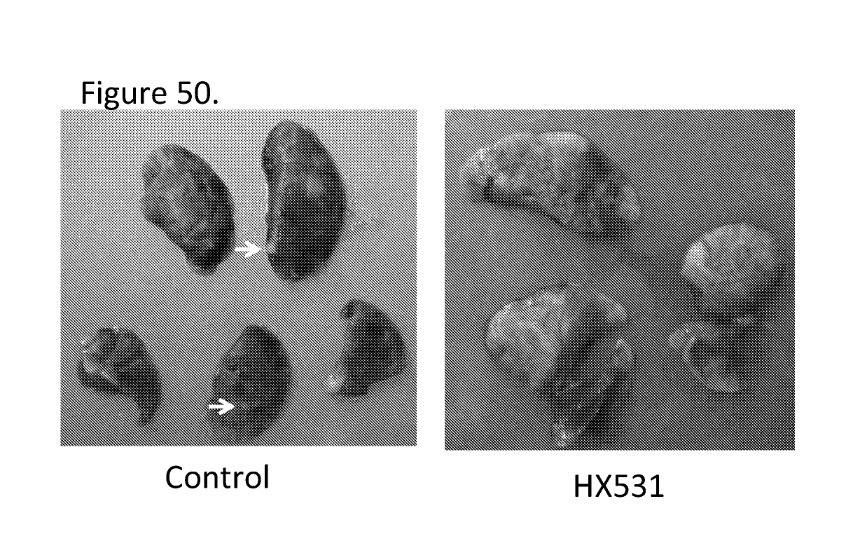

The effects of RXRG antagonists in lung cancer were evaluated based on the mouse model described above. FIG. 50 showed RXRG antagonist HX531 suppress lung cancer formation after tail vain injection of A549 NSCLC cells in nude mice. Two months after tail vein injection of one million A549 cells, lung cancer nodules were detected on lung surface in control group (FIG. 50, control), but not in HX531 treated group (FIG. 50, HX531, 100 ug/ml in drinking water). No significant side effects were detected after 2 months treatment of HX531.

### Example 7

### Test of RXRG Antagonist in In Vivo Cancer Models: Colon and Pancreatic Cancer

Spleen Injection of Colon and Pancreatic Cancer Cells:

We test the effects of RXR antagonist HX531 on pancreatic and colon cancer in vivo. Xenografts are performed mice, in some cases, on 6-week-old male athymic (nude) mice. After the nude mice are let to adapt the new environment for several days upon arrival. Luciferase labeled colon cancer cells HCT116 and HCT15 are collected from the culture medium by the trypsin, dissociated by pipetting, and resuspended in the above medium at 1×10⁷ cells/ml and held on ice. Researchers need to wear clean jumpsuit, mask, surgical gloves, and head cover. Prepare the animal by removing hair from the surgical site and clean with alcohol. Perform this procedure in an area separate from where the surgery is to be conducted. Surgeons will wash and dry their hands before aseptically donning sterile surgical gloves. The instruments will be autoclaved before injection. During injection, the instruments are sterilized by merging in the alcohol and rinsed with sterile PBS. For xenografts, mice are anaesthetized isoflurane inhalation. The animal is maintained in a surgical plane of anesthesia throughout the procedure by isoflurane inhalation. For colon cancer and pancreatic cancer cell spleen injection, mouse will be stabilized by sticky tape. The local skin will be cleaned by 70% alcohol. Middle superior abdomen longitudinal incision (1 cm) will be made into the peritoneal cavity. Spleen will be exposed and a thread loop will be put on the inferior end of spleen and the thread loop tightened. 1-2×10⁶ HCT116 or PC931019 cells in 200 μl medium are injected through the center of the loop at the inferior end of spleen, using a 31 gauge needle. Put spleen back to cavity and close the incision by two layer closure. Inner layer will be closed by Polyglycolic acid (Dexon®) suture, and skin will be closed by Stainless Steel wound clips. During surgery, monitor and/or maintain the animal's vital signs. After closure of wound, the skin will be cleaned by 70% alcohol. After surgery, move the animal to a warm, dry area and monitor it during recovery. Return the animal to its routine housing after it has fully recovered from anesthesia. Provide analgesics, 0.05-0.1 μg/g Buprenorphine, right after surgery and twice a day after surgery for 2 days. Remove skin closures 10 to 14 days post-operatively.

Effectiveness is tested at different dosages, for example, 16.7 μg/g/d HX531 (99.7 μg/ml in drinking water suspension, according to mouse can drink 15% water each day, to reach 10-20 μM in mouse body), 15 μg/g UVI3003 (89.9 μg/ml in drinking water), 15 μg/g C43 (89.9 μg/ml in drinking water), 12 μg/g/d bexarotene (71.86 μg/ml in drinking water) and control (DMSO) for 1 month. KRAS (A549) and EGFR mutant (H1650). KRAS mutant pancreatic cancer lines PC931019 and PC931102 have multiple (for example, 10) groups. Each group needs several nude mice; in some cases, 12.

Before xenograft, the pancreatic cancer lines PC931019 and PC931102 may be labeled with lentiviral luciferase expression and selected by hygromycin. Tumor formation will be monitored by luciferase imaging. Mice health will be checked every two days. The mice will be sacrificed by CO₂ or cardiac perfusion after they are obviously sick; and liver, lungs, brain, spleen, and other organs will be extracted. The weight of the livers and tumor mass will be measured and samples will be embedded in paraffin and immunostaining will be performed on sections to test the HDM2, HDMX, p53, RXRG, TRB1, TRB2, Cyclin E, Emi1, CDC25C, SKP2, p130, P27, pMEK, and pERK expression. TUNEL assay will be performed on sections to check the apoptosis after treatment. Total RNA will be isolated from the tumors and qPCR will be performed to check the HDM2, HDMX, SKP2, and E2F1 expression using human specific primers. Kaplan-Meiyer survival curve will be generated for comparison of treatment effectiveness. Mouse blood and some mouse organs such as brain, kidney, and liver will be collected and frozen to check the drug concentration and metabolism.

### Example 8

### Test Synergistic Effects Between RXR HX531 and AZD6244 in Mice

PC931019 is grafted in nude mice. There are 4 groups: HX531, AZD6244, HX531+AZD6244, and control groups. Each group needs 15 mice, so we need 60 mice for this study. After treatment, cells are tested according to above methods. After treatment, mice are tested according to above methods.

### Example 9

### Test the Effectiveness of the RXRG Antagonist for the Treatment of Pancreatic Cancer in Transgenic Mice

LSL-Kras^{G12D/+}; LSL-Trp53^{R172H/+}; Pdx-1-Cre mice, which can develop metastatic PDA is proved by David Tuveson through collaboration. The effectiveness on pancreatic cancer transgenic mouse model is tested at different dosage, for example, 16.7 ug/g/d HX531 (99.7 ug/ml in drinking water suspension, according to mouse can drink 15% water each day, to reach 10-20 uM in mouse body), 15 ug/g UVI3003 (89.9 ug/ml in drinking water), 12 ug/g/d bexarotene (71.86 ug/ml in drinking water) and control (DMSO) for 2 month. After treatment, mice will be tested according to above methods.

Flow charts for pancreatic cancer xenograft:

Nude mice>>stabilization>>anesthesia>>spleen injection of pancreatic cancer cells>>add drugs next day>>Observe the tumor formation by luciferase imaging every two weeks>>weigh the weight of mice once a week>>euthanize by CO₂ or perfusion>>get organs such as livers>>weigh the livers>>Freezing in dry ice or fixation by paraformaldehyde>>histology analysis of tumor and RNA isolation>>analysis of gene expression and survival curve>>TUNEL assay to check the apoptosis>>drug concentration and metabolism assay.

Potential, Non-Limiting, Mechanism of RXRG Antagonists.

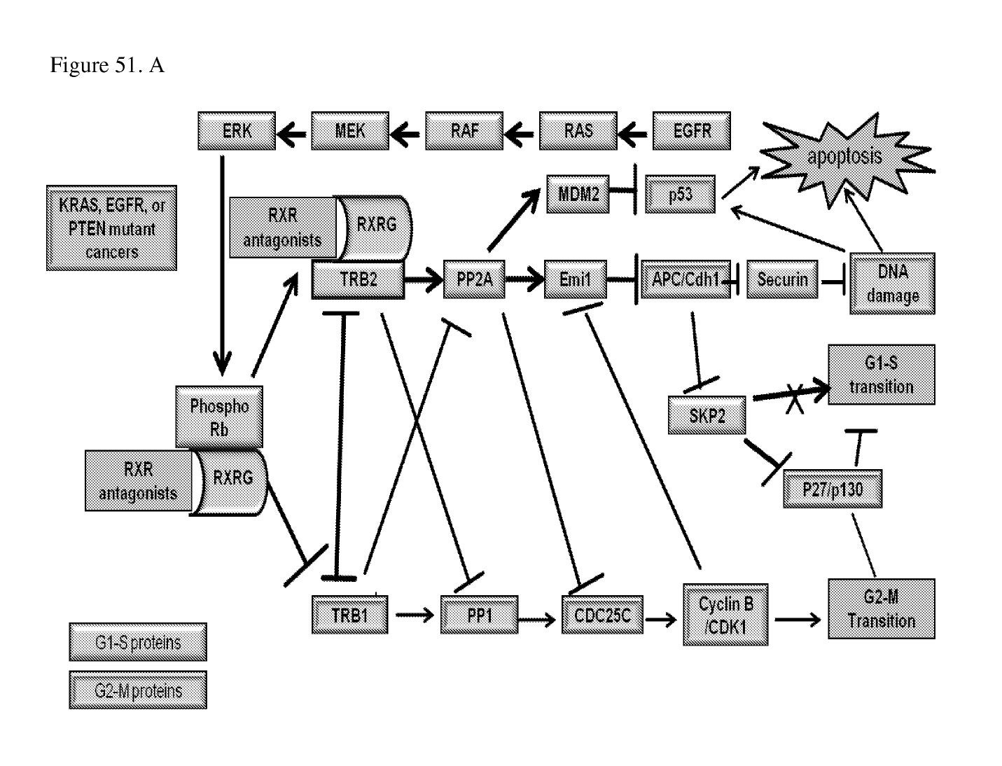

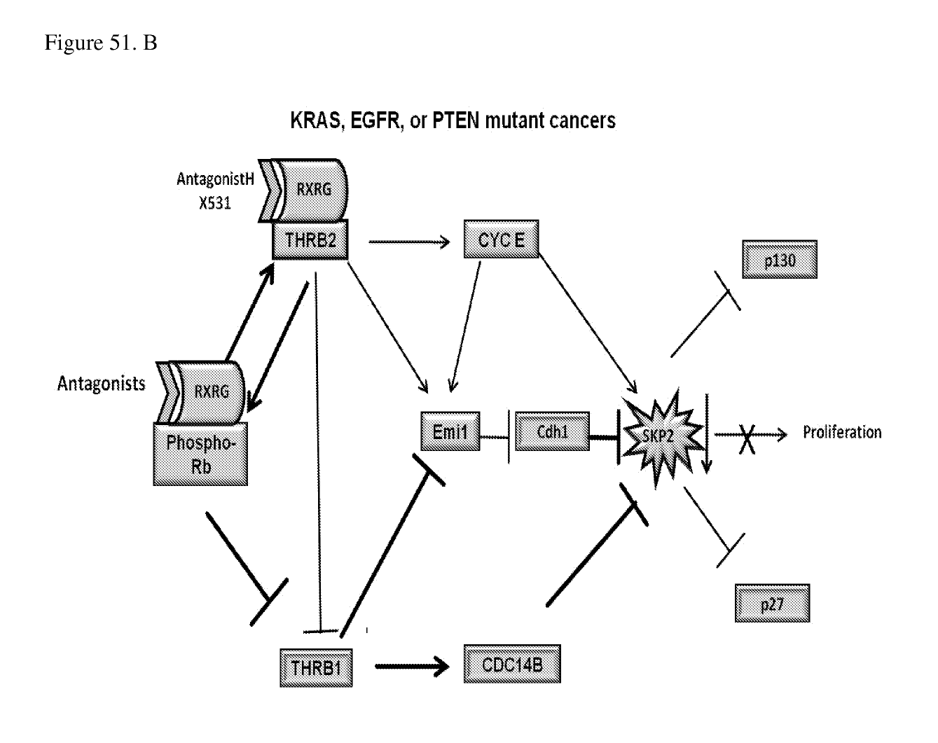

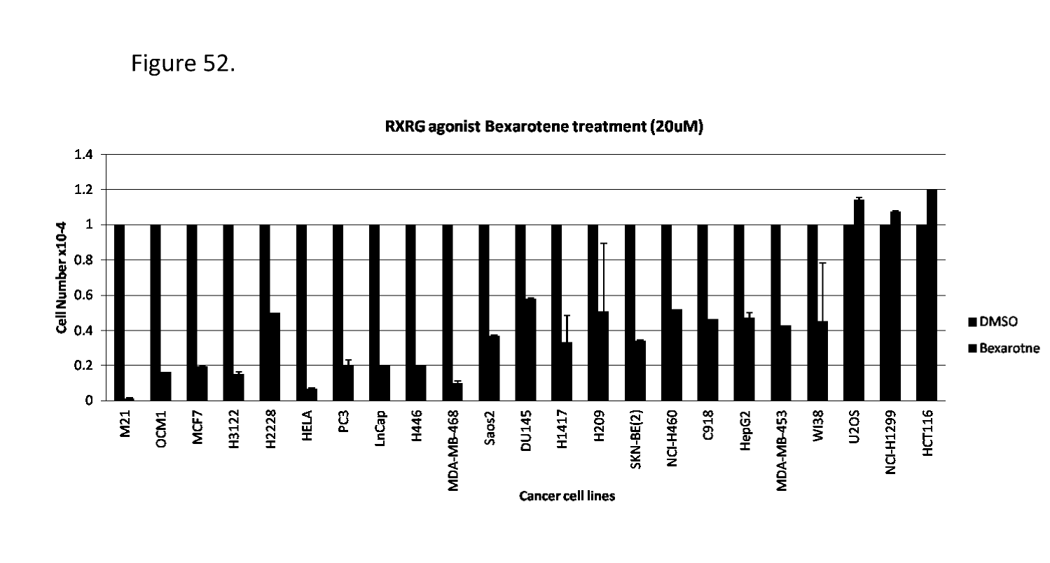

Without wishing to be bound by any theory, we propose a potential, non-limiting mechanism of RXRG antagonist in KRAS, EGFR, or PTEN mutated cancers as illustrated in FIGS. 51A and 51B: RXRG antagonists target S-phase promoting complex, which comprises phospho-Rb family proteins, TRB2, RXRG, Cyclin E, PP2A, and Emi1, and is important for Emi1 and SKP2 activation, APC/cdh1 inactivation, and S phase progression; KRAS or EGFR activation or PTEN inactivation promotes Rb hyperphosphorylation and S phase promoting complex formation, which leads to cell proliferation; RXRG maintains Rb hyperphosphorylation and S phase promoting complex formation in cancers; RXRG antagonists suppress KRAS, EGFR, or PTEN mutated cancer growth by promoting Rb dephosphorylation, S phase promoting complex dissociation, APC/cdh1 activation, SKP2 degradation, and G1 arrest; APC/cdh1 activation cause securin degradation and sister chromatid early separation, leading to DNA damage and apoptosis; RXRG antagonists also suppress KRAS, EGFR, or PTEN mutant cancer growth by activation of p53 and induction of apoptosis. Any theoretical mechanism or theory described above and herein or in any portion of this application is without the intention to limit the scope of the present invention.

### 3. RXRG Agonists for Treatment of Retinoblastoma

### Example 10

### RXRG Agonist Suppressed Cell Growth

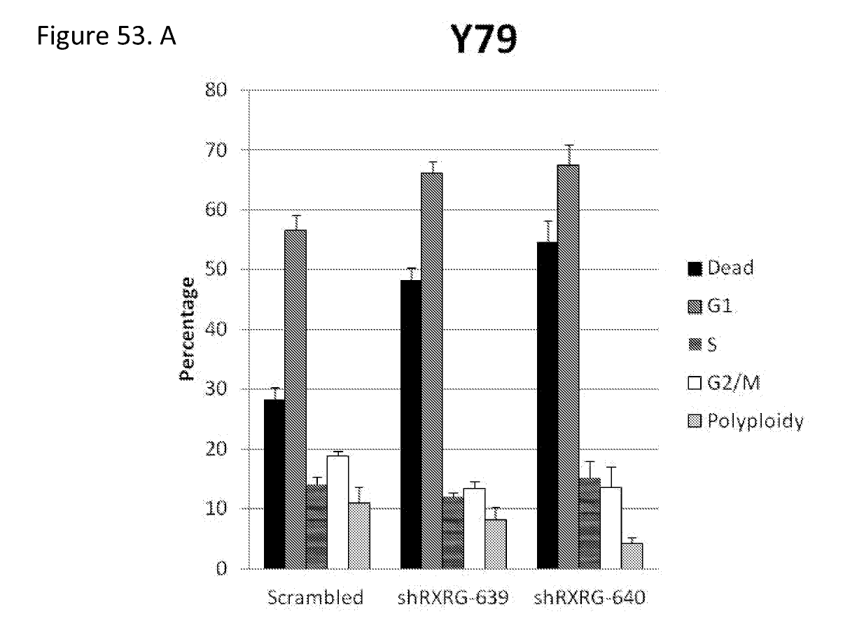

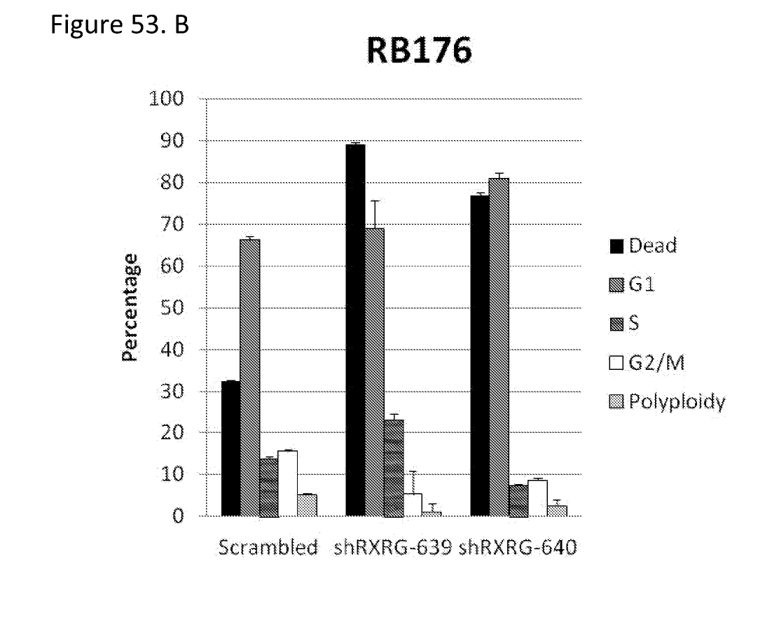

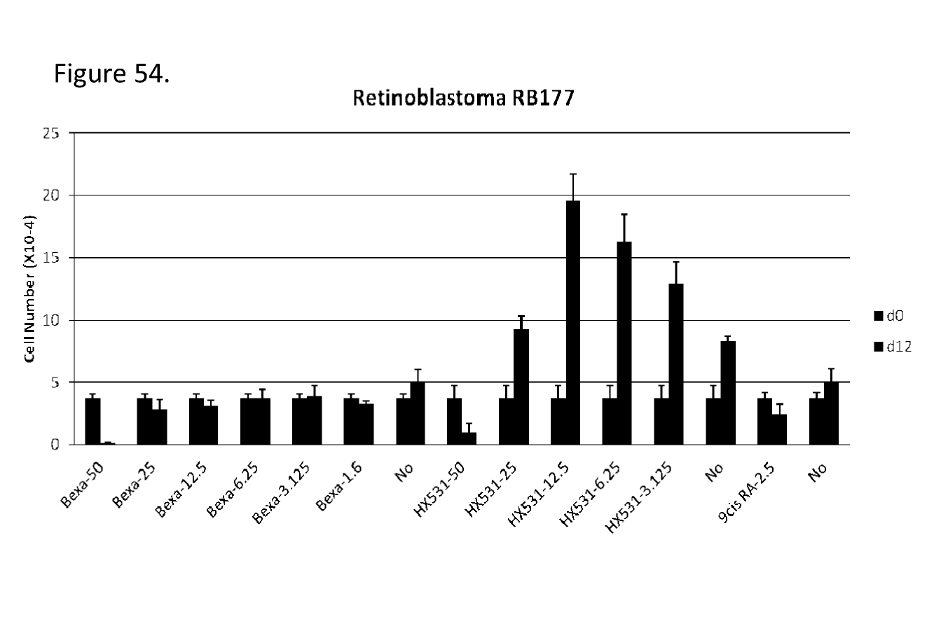

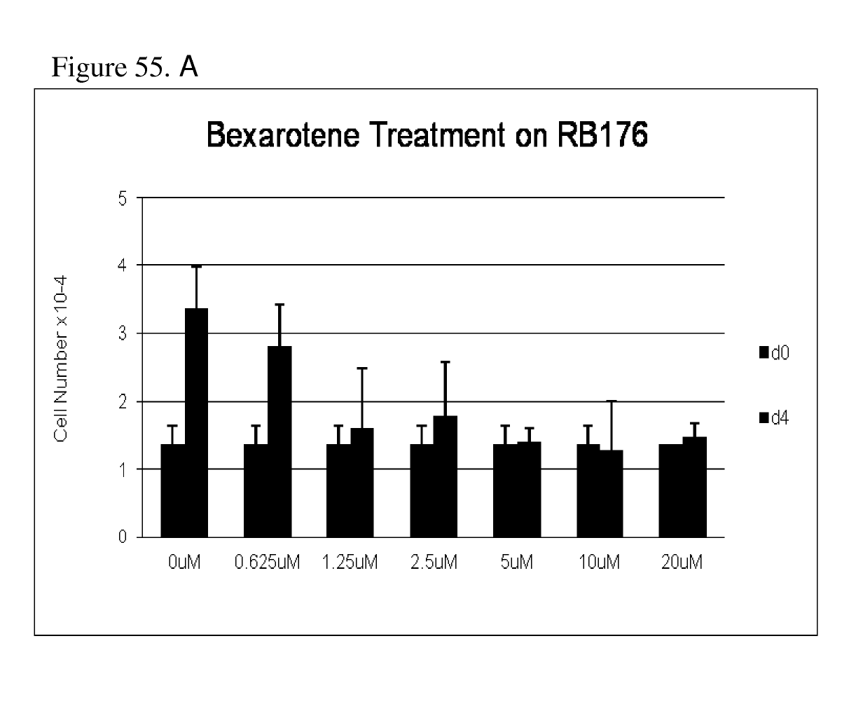

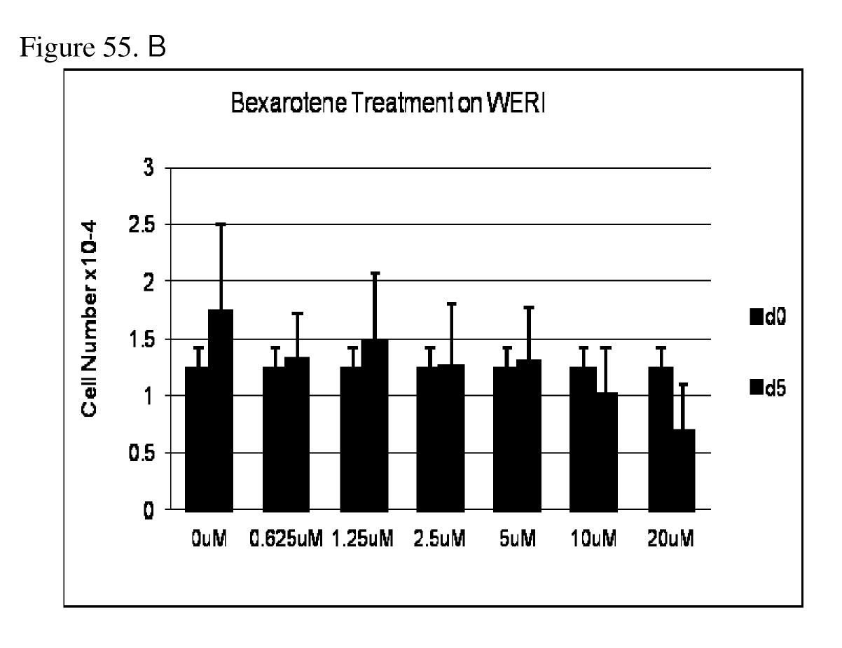

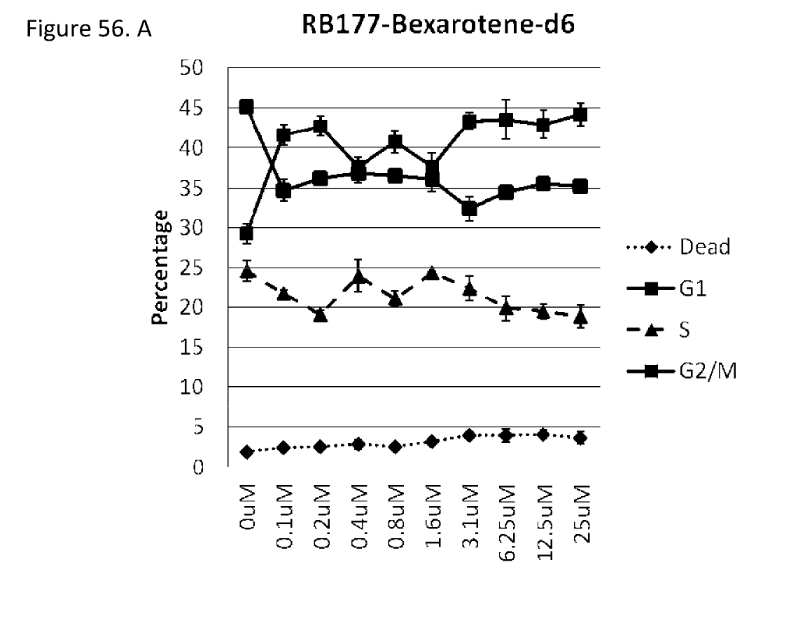

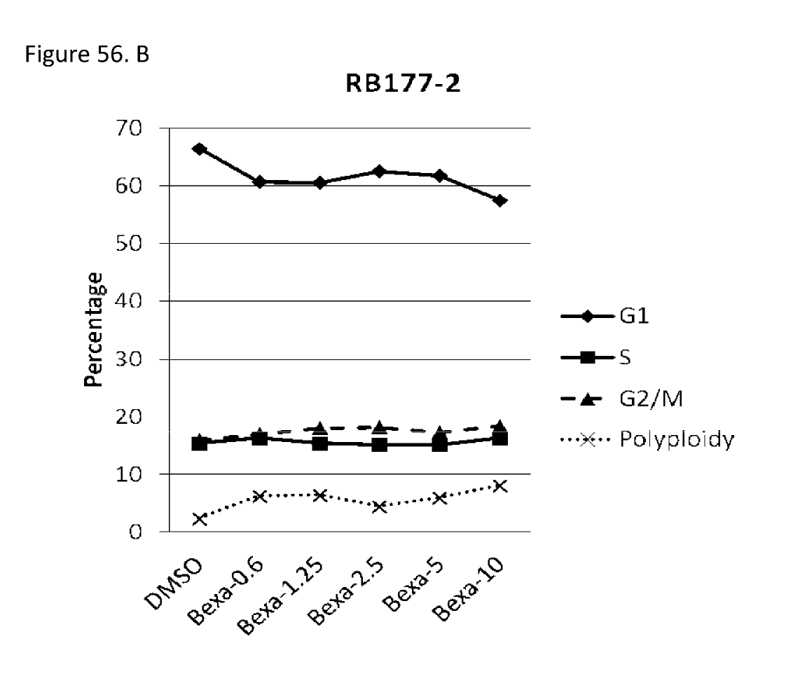

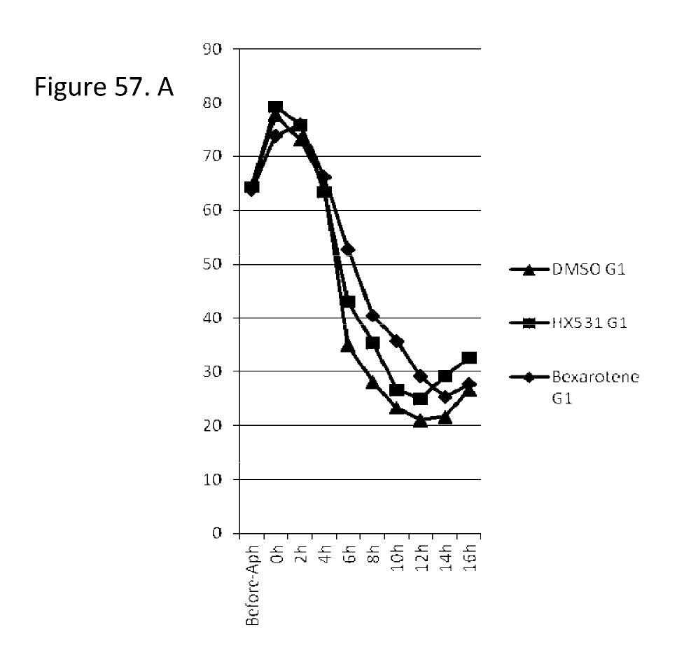

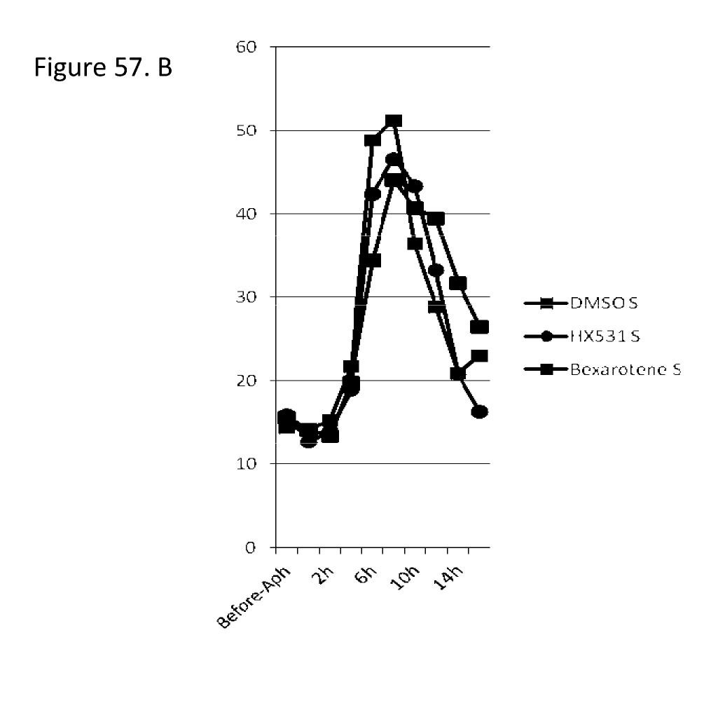

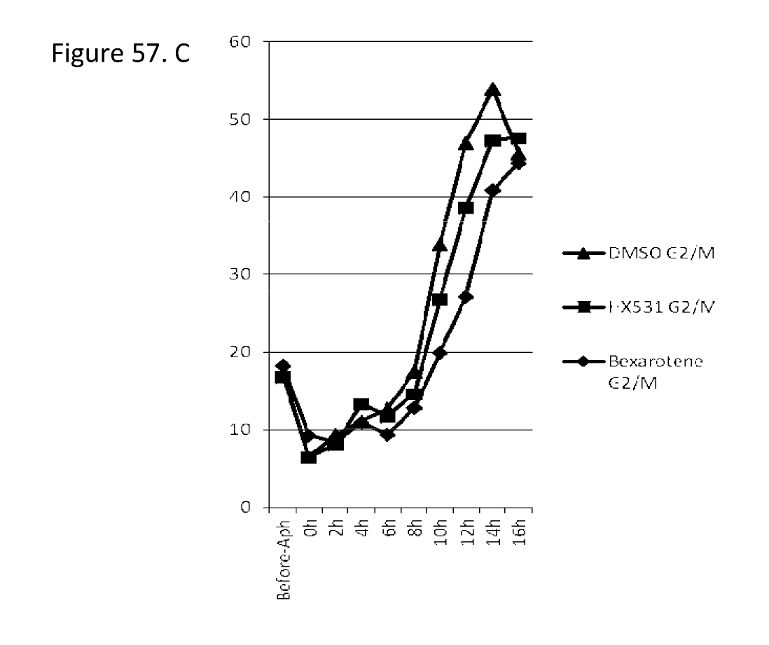

We found that LKO lentivirus-mediated RXRG knockdown in retinoblastoma cells Y79 and RB176 suppressed G1/S transition demonstrated by PI staining and cell cycle. Treatment of retinoblastoma cell RB177 with RXRG agonists Bexarotene and 9-cis-Retinoic acid led to suppressed cell growth (FIG. 53). Test of one RXRG agonist, bexarotene, showed that it suppressed the growth of multiple cell lines. While treatment of RXRG agonists bexarotene and 9 cis RA led to suppressed retinoblastoma cell growth, treatment with RXRG antagonist HX531 promoted retinoblastoma cell growth at low dosages (FIG. 54). Growth suppression by Bexarotene on RB176 and WERI cells were also observed (FIG. 55). Cell cycle analysis demonstrated that Bexarotene caused G2-M block and polyploidy in RB 177 (FIG. 56). Cell cycle synchronization and cell cycle analysis in RB177 after treatment with 10 uM bexarotene and HX531 for 2 days showed Bexarotene treatment caused delayed S phase progression and delayed G2/M transition (FIG. 57).

### Example 11

### RXRG Agonist Activated p53-Targeted Gene Expression

Real Time PCR Analysis:

Total RNA was isolated from puromycin-selected cells using RNeasy Mini Kit (Qiagen) or GenElute™ Mammalian Total RNA Miniprep Kit (Sigma). RNA reverse transcription was performed with ImProm-II™ Reverse Transcription System (Promega). Primers were designed by Beacon Designer software (Premier Biosoft International) or web-based Primer3 (http://frodo.wi.mit.edu/primer3/). Relative mRNA levels were determined by qPCR using QuantiTect SYBR Green PCR Kit (Qiagen) or Fermentus Maxima® SYBR Green qPCR Master Mix on an Applied Biosystems ABI 7900HT Sequence Detection System. Two samples for each group were collected and evaluated in triplicate and normalized to β-actin mRNA quantitated in parallel. Program conditions: activation, 95° C. 10 min; amplification, 40 cycles (denaturation 95° C. 20 sec, annealing 54° C. 30 sec, extension 72° C. 30 sec). qPCR primers are listed in Table 7.

TABLE 7
Sequences of qPCR primers.

| Primers | Sense | Antisense |
|---|---|---|
| HDM2 | ggtgaggagcaggcaaatgtg | gctggaatctgtgaggtggttac |
| GADD45A | cgaggacgacgacgaagatg | cgcaggatgttgatgtcgtt |
| SKP2 | ctagcgtctgatgagtctctatgg | gggcagcggaaggcaatc |
| RXRG | agcgatgaccactcttgttag | tcgtcagttcatgttcctctc |
| CDKN1A(p21) | cccctttcctggacactcag | caccctgcccaaccttagag |
| CDKN1B(p27) | gctccggctaactctgagga | aagaatcgtcggttgcaggt |
| RBI | ccagtaccaaagttgataatgc | ccaagaaacttttagcaccaatgcag |
| 14-3-3 | gagccatggagagagccagt | agagcaggtttcgctcttcg |
| β-Actin | gcaagcaggagtatgacgagtc | caagaaagggtgtaacgcaactaag |

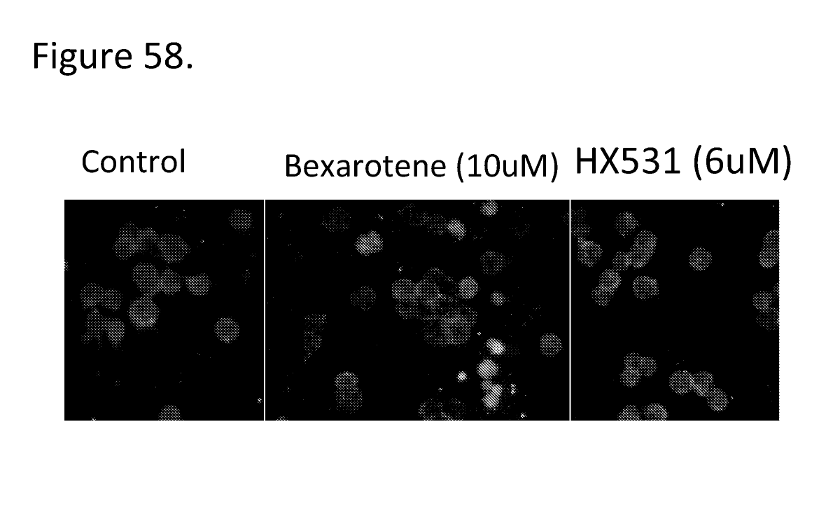

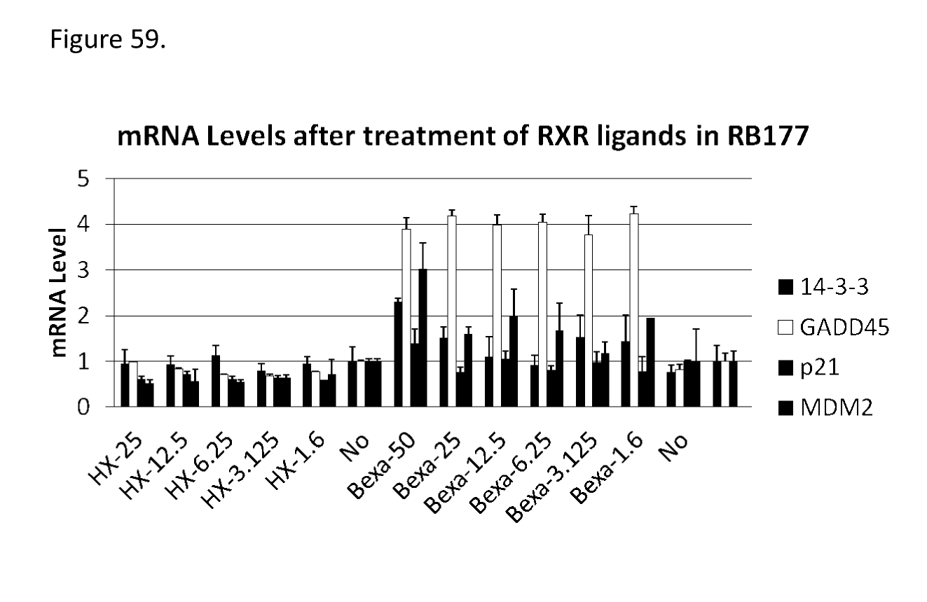

Bexarotene activated p53 in RB177 on Day 2 of treatment, as shown in FIG. 58. Analysis of mRNA level showed that expression of p53-targeted genes such as 14-3-3, GADD45, p21, and MDM2 in RB177 was promoted by Bexarotene, a RXRG agonist. HX531, a RXRG antagonist, did not promote expression of these genes (FIG. 59).

### Example 12

### Test of RXRG Agonist in In Vivo Cancer Models

Protocol for Retinoblastoma Xenograft:

To test the effects of RXR agonists on retinoblastoma in vivo, Sub-retinal injection of retinoblastoma cells is performed according to protocol (MacLaren et al., Nature, 2006 444(7116): 203-207; Xu et al, Cell, 2009, 137(6): 1018-1031). Xenografts is performed on 6-week-old male athymic (nude) mice (Taconic, Hudson, N.Y.). After the nude mice come to the facility, we let these mice adapt the new environment for several days. Retinoblastoma cells are collected from the culture medium, dissociated by pipetting, and resuspended in the above medium at 1×10(5) cells/μl and held on ice. For xenografts, mice are anaesthetized by intra-peritoneal injection of a ketamine (final concentration; 10 mg/ml) and xylazine (final concentration; 1 mg/ml) mixture (0.015 ml/g mouse weight), and with Alcaine (proparacaine HCL) ocular surface anesthesia. The mouse is stabilized by paper tape. The instruments are autoclaved before injection. During injection, the instruments are sterilized by merging in the alcohol and rinsed with sterile PBS. Under a surgical microscope, a 30 gauge sharp needle is used to make two holes through the sclera of eye, one into the intravitreal space to reduce intraocular pressure; and one tangentially through the sclera into the sub-retinal space for injection (MacLaren et al., Nature, 2006 444(7116): 203-207). 2×10(5) cells in 2 μl medium are injected through the second hole into the sub-retinal space of eye, using a 1.5 cm, 33 gauge blunt end microinjection needle (7803-05, Hamilton, Reno, Nev.). During the injection, we visualize the subretinal injection by looking into the eye. We gently push the blunt needle to the inner side of sclera and choroid to ensure that the needle tip is between retina and choroid. In this way the cells are injected into subretinal space. After injection, eyes are covered with ophthalmic bacitracin ointment. Mice are put into new cages with some sterile cotton or stripped papers to keep them warm. Mice are monitored for activity for several hours after the subretinal inoculation, and sent back to racks after they are awake. Buprenorphine is SQ injected right after xenograft and twice a day within 48 hours after cell injection. If the tumor in eyeball is more than 0.5 cm, we euthanize the mice. We observe the mice behavior such as movement, tears, and irritation. Buprenorphine is administrated subcutaneously for surveillance of pain twice per day if the eye is swelling. If there are severe pain and distress we sacrifice the mice. Generally, we sacrifice the mice about 2 months after injection.

After xenograft, we test the Bexarotene effectiveness on retinoblastoma xenografts. After injection, the Bexarotene is added to the drinking water (Bexarotene: 7 ug/g body weight, 47 ug/ml in drinking water, according to that each mouse drinks water volume about 15% of their body weight). The 9cis RA and Bexarotene are administrated according to regulations of bio-safety level II. Their powder is dissolved in DMSO and PBS for stock solution.

Tumor formation is monitored by observation. After two months, or the eye size is more than 5 mm, the mice are sacrificed by CO₂ and eyes extracted. The weight of the eyes and tumor mass are measured and eyes are embedded in paraffin and immunostaining is performed on sections to test the HDM2, HDMX, p53, RXRG, THRB1, THRB2, Cyclin E, Emi1, CDCl₄B, SKP2, and MycN expression. TUNEL assay is performed on sections to check the apoptosis after treatment. Total RNA is isolated from the tumors and qPCR is performed to check the HDM2, HDMX, SKP2, E2F1, and MycN expression using human specific primers.

### Example 13

### RXRG Agonist Suppressed RB117 Growth in Mice

We tested the effects of RXR agonists on retinoblastoma in vivo. Sub-retinal injection of retinoblastoma cells was performed according to our approved protocol (10-06-010) (modified from MacLaren et al., Nature, 2006 444(7116): 203-207; Xu et al, Cell, 2009, 137(6): 1018-1031). 6-week-old male athymic (nude) mice (Taconic, Hudson, N.Y.) were used for sub-retinal injection.

For subconjunctival injection, Bexarotene was prepared in the solvent with concentration of 50 mmol/L. 10 μl of Bexarotene in solvent of 15% polypropylene glycol (pPPG), 10% propylene glycol (PPG), 5% cremophor eL, and 70% PBS was subconjunctivally injected for each eye. Bexarotene oral drinking (7 ug/g/day, 47 ug/ml in drinking water, to reach 10 μM in mouse body) was also tested.

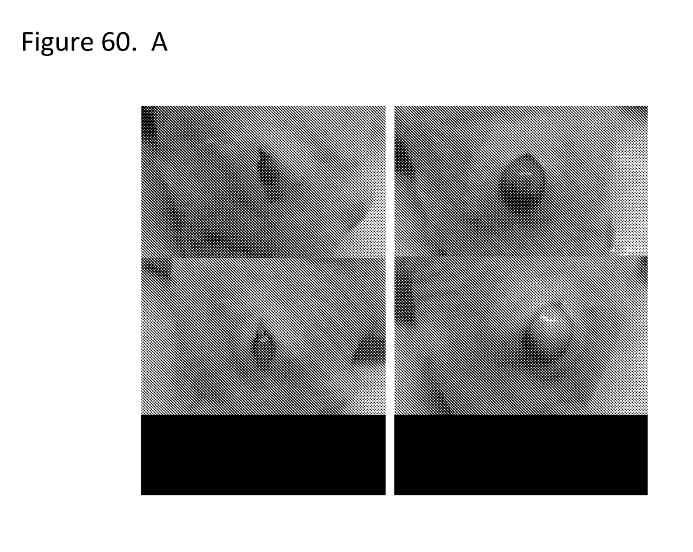

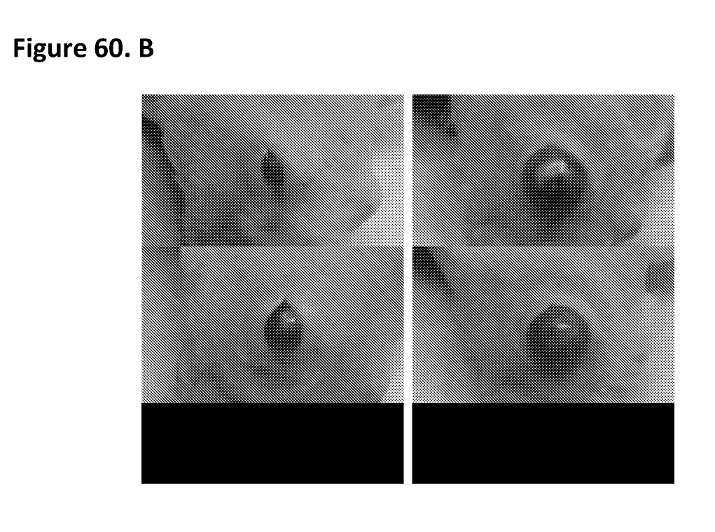

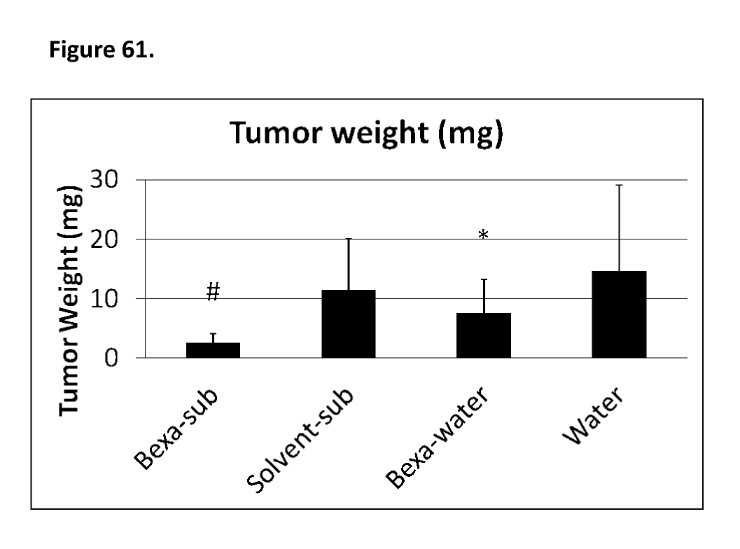

As illustrated by FIG. 60, both subconjunctival and oral administration of Bexarotene significantly suppressed retinoblastoma growth in subretinal grafted mouse animal model. The tumor weight was significantly lower in Bexarotene treated mice than in control (FIG. 61).

Potential, Non-Limiting, Mechanism of RXRG Agonists.

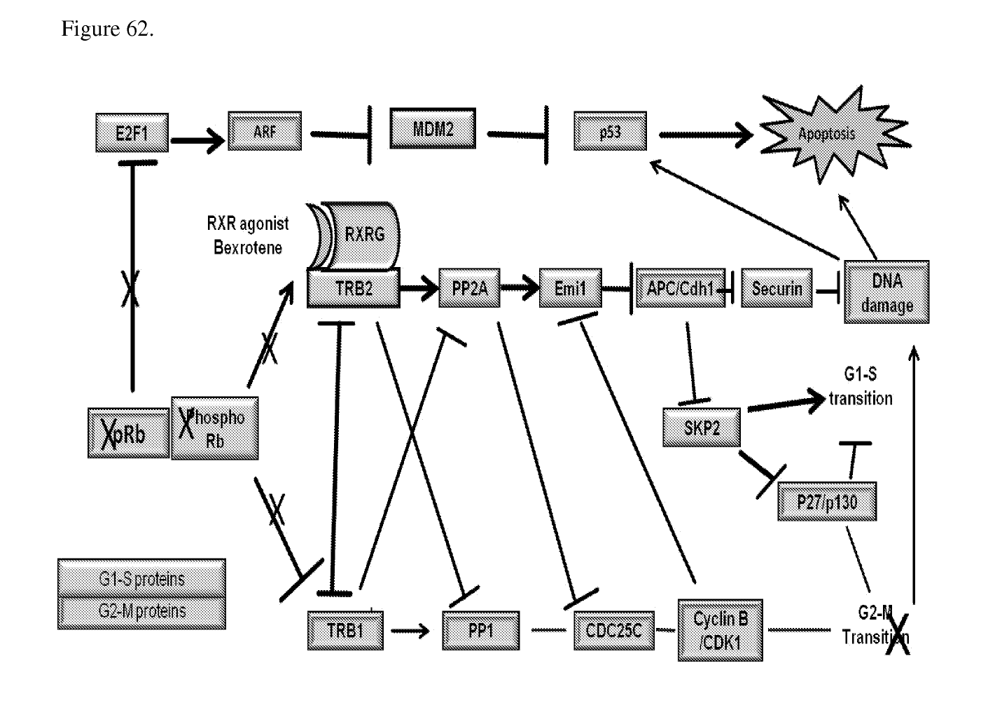

Without wishing to be bound by any theory, we propose a potential, non-limiting mechanism of RXRG agonist in retinoblastoma as illustrated in FIG. 62: RXRG agonists such as Bexarotene promote TRB2 activity and G1-S transition, but such agonists cause G2-M block, resulting in cell cycle arrest in retinoblastoma cells; G2-M block and stabilized securin lead to DNA damage and apoptosis.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

What is claimed is:

1. A method for treating cancer, comprising the step of administering to a subject suffering from or susceptible to cancer a therapeutically effective amount of a compound of formula I:
wherein,
R¹ is hydrogen or an optionally substituted C₁₋₁₂ aliphatic group;
each R² is independently halogen, R′, —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —N(R)C(O)N(R)₂, —N(R)SO₂N(R)₂, —N(R)SO₂R, —OC(O)N(R)₂, or an optionally substituted C₁₋₁₂ aliphatic group, or two R² groups on adjacent carbon atoms are taken together with their intervening atoms to form an optionally substituted 5- to 7-membered ring having 0-4 heteroatoms selected from nitrogen, oxygen, or sulfur;
each R³ is independently halogen, R′, —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —OSO₂R, —N(R)C(O)N(R)₂, —N(R)SO₂N(R)₂, —N(R)SO₂R, —OC(O)N(R)₂, or an optionally substituted C₁₋₁₂ aliphatic group, or two R³ groups on adjacent carbon atoms are taken together with their intervening atoms to form an optionally substituted 5- to 7-membered ring having 0-4 heteroatoms selected from nitrogen, oxygen, or sulfur;
m is from 0 to 4, inclusive;
p is from 0 to 4, inclusive;
T is a covalent bond or an optionally substituted, bivalent C₁₋₆ saturated or unsaturated, straight or branched, hydrocarbon chain, wherein one or two methylene units of T are optionally and independently replaced by —Cy-, —C(R)₂—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Cy is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
X is a covalent bond, —O—, —NR—, —NR³—, —NCH₂R³—, —C(R)₂—, —C(═CH₂)—, —CHR³—, —C(R³)₂—, or —S—;
each R is independently hydrogen or R′;
each R′ is independently an optionally substituted group selected from C₁₋₁₀ aliphatic, phenyl, a 3-7 membered saturated or partially unsaturated carbocyclic ring, a 3-7 membered saturated or partially unsaturated monocyclic heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur; or:
two R′ groups on the same nitrogen are taken together with their intervening atoms to form an optionally substituted 3-7 membered saturated, partially unsaturated, or heteroaryl ring having 1-4 heteroatoms independently selected from nitrogen, oxygen, or sulfur;
or a pharmaceutically acceptable salt or pharmaceutical composition thereof.

2. A method for treating cancer, comprising the step of administering to a subject suffering from or susceptible to cancer a therapeutically effective amount of a compound of formula formula VI, VII or VIII:
wherein:
R¹ is hydrogen or an optionally substituted C₁₋₁₂ aliphatic group;
each R³ is independently halogen, R′, —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —OSO₂R, —N(R)C(O)N(R)₂, —N(R)SO₂N(R)₂, —N(R)SO₂R, —OC(O)N(R)₂, or an optionally substituted C₁₋₁₂ aliphatic group, or two R³ groups on adjacent carbon atoms are taken together with their intervening atoms to form an optionally substituted 5- to 7-membered ring having 0-4 heteroatoms selected from nitrogen, oxygen, or sulfur;
X is a covalent bond, —O—, —NR—, —NR³—, —NCH₂R³—, —C(R)₂—, —C(═CH₂)—, —CHR³—, —C(R³)₂—, or —S—;
each R is independently hydrogen or R′;
each R′ is independently an optionally substituted group selected from C₁₋₁₀ aliphatic, phenyl, a 3-7 membered saturated or partially unsaturated carbocyclic ring, a 3-7 membered saturated or partially unsaturated monocyclic heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur; or:
two R′ groups on the same nitrogen are taken together with their intervening atoms to form an optionally substituted 3-7 membered saturated, partially unsaturated, or heteroaryl ring having 1-4 heteroatoms independently selected from nitrogen, oxygen, or sulfur; and
each Y is independently ═CH— or ═N—;
or a pharmaceutically acceptable salt or pharmaceutical composition thereof.

3. A method for treating cancer, comprising the step of administering to a subject suffering from or susceptible to cancer a therapeutically effective amount of a compound of formula XI:
wherein,
R¹ is hydrogen or an optionally substituted C₁₋₁₂ aliphatic group;
each R² is independently halogen, R′, —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —N(R)C(O)N(R)₂, —N(R)SO₂N(R)₂, —N(R)SO₂R, —OC(O)N(R)₂, or an optionally substituted C₁₋₁₂ aliphatic group, or two R² groups on adjacent carbon atoms are taken together with their intervening atoms to form an optionally substituted 5- to 7-membered ring having 0-4 heteroatoms selected from nitrogen, oxygen, or sulfur;
each R³ is independently halogen, R′, —NO₂, —CN, —OR, —SR, —N(R)₂, —C(O)R, —CO₂R, —C(O)C(O)R, —C(O)CH₂C(O)R, —S(O)R, —S(O)₂R, —C(O)N(R)₂, —SO₂N(R)₂, —OC(O)R, —N(R)C(O)R, —N(R)N(R)₂, —N(R)C(═NR)N(R)₂, —C(═NR)N(R)₂, —C═NOR, —OSO₂R, —N(R)C(O)N(R)₂, —N(R)SO₂N(R)₂, —N(R)SO₂R, —OC(O)N(R)₂, or an optionally substituted C₁₋₁₂ aliphatic group, or two R³ groups on adjacent carbon atoms are taken together with their intervening atoms to form an optionally substituted 5- to 7-membered ring having 0-4 heteroatoms selected from nitrogen, oxygen, or sulfur;
m is from 0 to 4, inclusive;
p is from 0 to 4, inclusive;
T is a covalent bond or an optionally substituted, bivalent C₁₋₆ saturated or unsaturated, straight or branched, hydrocarbon chain, wherein one or two methylene units of T are optionally and independently replaced by —Cy-, —C(R)₂—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Cy is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
X is a covalent bond, —O—, —NR—, —NR³—, —NCH₂R³—, —C(R)₂—, —C(═CH₂)—, —CHR³—, —C(R³)₂—, or —S—;
each R is independently hydrogen or R′;
each R′ is independently an optionally substituted group selected from C₁₋₁₀ aliphatic, phenyl, a 3-7 membered saturated or partially unsaturated carbocyclic ring, a 3-7 membered saturated or partially unsaturated monocyclic heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur; or:
two R′ groups on the same nitrogen are taken together with their intervening atoms to form an optionally substituted 3-7 membered saturated, partially unsaturated, or heteroaryl ring having 1-4 heteroatoms independently selected from nitrogen, oxygen, or sulfur;
or a pharmaceutically acceptable salt or pharmaceutical composition thereof.

4. The method of claim 1, 2, or 3, wherein the cancer does not have EGFR or EGFR mutation, and the compound is a RXRG agonist.

5. The method of claim 4, wherein the compound is bexarotene.

6. The method of claim 4, wherein the cancer is retinoblastoma.

7. The method of claim 4, wherein the compound is bexarotene, and the cancer is retinoblastoma.

8. The method of claim 1, 2, or 3, wherein the cancer has a EGFR, KRAS, BRAF, or PTEN mutation, and the compound is a RXRG antagonist.

9. The method of claim 8, wherein the compound is HX531, UVI3003 or PA 452.

10. The method of claim 9, wherein the compound is HX531.

11. The method of claim 8, wherein the cancer has EGFR or KRAS mutation and the cancer is non-small cell lung cancer, pancreatic cancer, gastric cancer, colon cancer, hepatoma, leukemia, or breast cancer.

12. The method of claim 8, wherein the cancer has PTEN mutation and the cancer is breast cancer, prostate cancer, small cell lung cancer or glioma.

13. The method of claim 8, wherein the cancer has BRAF mutation and the cancer is melanoma.

14. The method of claim 8, wherein the cancer is non-small cell lung cancer with EGFR or KRAS mutation, and the compound is HX531.

15. The method of claim 8, wherein the cancer is pancreatic cancer with EGFR or KRAS mutation, and the compound is HX531.

16. A method of inhibiting growth of cancer cells without KRAS or EGFR mutations with a retinoid X receptor gamma (RXRG) agonist, wherein the agonist is of formula I, II, III, IV, V, VI, VII, VIII, or XI.

17. A method of promoting apoptosis of cancer cells without KRAS or EGFR mutations with retinoid X receptor gamma (RXRG) agonist, wherein the agonist is of formula I, II, III, IV, V, VI, VII, VIII, or XI.

18. A method of inhibiting proliferation of cancer cells without KRAS, EGFR or PTEN mutations with retinoid X receptor gamma (RXRG) agonist, wherein the agonist is of formula I, II, III, IV, V, VI, VII, VIII, or XI.

19. A method of delaying S phase progression and/or G2/M transition in cancer cells without KRAS or EGFR mutations with retinoid X receptor gamma (RXRG) agonist, wherein the agonist is of formula I, II, III, IV, V, VI, VII, VIII, or XI.

20. The method of claim 16, 17, 18 or 19, wherein the compound is Bexarotene.

21. A method of inhibiting growth of cancer cells with KRAS, EGFR or PTEN mutations with retinoid X receptor gamma (RXRG) antagonist, wherein the antagonist is of formula I, II, III, IV, V, VI, VII, VIII, or XI.

22. A method of promoting apoptosis of cancer cells with KRAS, EGFR or PTEN mutations with retinoid X receptor gamma (RXRG) antagonist, wherein the antagonist is of formula I, II, III, IV, V, VI, VII, VIII, or XI.

23. A method of inhibiting proliferation of cancer cells with KRAS, EGFR or PTEN mutations with retinoid X receptor gamma (RXRG) antagonist, wherein the antagonist is of formula I, II, III, IV, V, VI, VII, VIII, or XI.

24. A method of suppressing G1/S transition in cancer cells with KRAS, EGFR or PTEN mutations with retinoid X receptor gamma (RXRG) antagonist, wherein the antagonist is of formula I, II, III, IV, V, VI, VII, VIII, or XI.

25. A method of modulating functions of Treprec-Xu complex in cancer by inhibiting or promoting association or dissociation of one or more components of the complex with each other and/or with the complex, comprising the step of treating the Treprec-Xu complex or at least one component thereof with a compound of formula I, II, III, IV, V, VI, VII, VIII, or XI.

26. A method of inhibiting Treprec-Xu complex in cancer by promoting dissociation of one or more components of the complex with each other and/or with the complex, comprising the step of treating the Treprec-Xu complex or at least one component thereof with a compound of formula I, II, III, IV, V, VI, VII, VIII, or XI.

27. The method of claim 21, 22, 23, 24, 25 or 26, wherein the compound is selected from HX531, UVI3003 and PA452.

28. The method of claim 27, wherein the compound is HX531.

29. The method of claim 1, 2, 3, 21, 22, 23, 24, 25 or 26, wherein the compound is selected from the compounds of Table 1.

30. The method of claim 1, 2, 3, 21, 22, 23, or 24, wherein the compound binds to RXRG to effect the dissociation of Rb and RXRG.

31. The method of claim 1, 2, 3, 21, 22, 23, or 24, wherein the compound administered promotes dissociation of one or more components of the Treprec-Xu complex.

32. The method of claim 31, wherein dissociation of the Treprec-Xu complex is effected by inhibiting the association of RXRG with another component of the complex.

33. The method of claim 32, wherein dissociation of the Treprec-Xu complex is effected by inhibiting the association of RXRG with Rb.

34. The method of claim 33, wherein dissociation of the Treprec-Xu complex is effected by inhibiting the association of RXRG with Phospho-Rb.

35. A method of dissociating the Treprec-Xu complex in a biological sample, comprising the step of treating the biological sample with a compound of formula I, II, III, IV, V, VI, VII, VIII, or XI.
